(19) United States
(12) Patent Application Publication
(10) Pub. No.: US 2026/0151360 A1
(43) Pub. Date: Jun. 4, 2026

(54) ENDOXIFEN COMPOSITIONS AND METHODS OF USE THEREOF AND SYNTHESIS THEREOF

(71) Applicant: Atossa Therapeutics, Inc., Seattle, WA (US)

(72) Inventor: Steven Carl QUAY, Seattle, WA (US)

(21) Appl. No.: 19/452,023

(22) Filed: Jan. 16, 2026

Related U.S. Application Data
(63) Continuation of application No. PCT/US2024/038753, filed on Jul. 19, 2024.
(60) Provisional application No. 63/527,801, filed on Jul. 19, 2023.

(51) Int. Cl. A61K 31/138; A61K 31/015; A61K 31/14; A61K 31/165; A61K 31/265; A61K 31/325; A61K 31/343

(52) U.S. Cl. CPC A61K 31/138; A61K 31/015; A61K 31/14; A61K 31/165; A61K 31/265; A61K 31/325; A61K 31/343

(57) ABSTRACT

Described herein are endoxifen compositions comprising novel compounds and methods of use thereof. Methods of synthesis for the compositions are described herein. The endoxifen compositions described herein may exhibit aromatase inhibition activity or estrogen receptor activity. The endoxifen compositions described herein may be useful in the treatment of a condition in a subject, such as cancer.

### CROSS-REFERENCE

This application is a continuation of International Application No. PCT/US2024/038753 filed Jul. 19, 2024 which claims the benefit of U.S. Provisional Application No. 63/527,801 filed Jul. 19, 2023, which are incorporated herein by reference in their entirety.

### BACKGROUND

Cancer is a group of diseases characterized by uncontrolled cell growth. Cancer can be deadly and while many treatment options exist, there is a need for continuous development of new treatment therapies and regimens. New pharmaceutical drugs can allow for treatment of wider ranges of cancers and be helpful for the treatment of current therapy resistant cancers and tumors. Hence, there remains a need for new active pharmaceutical ingredients (APIs) and compositions thereof that act as anti-tumor compounds and methods of making them.

### SUMMARY

In various aspects, the present disclosure provides a composition comprising: an endoxifen

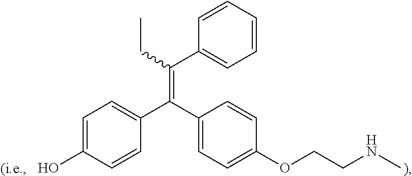

or a pharmaceutically acceptable salt or tautomer thereof; and not less than 0.01 and not more than 10% w/w of a further component, wherein the further component comprises one or more of the following compounds, or a pharmaceutically acceptable salt or tautomer thereof:

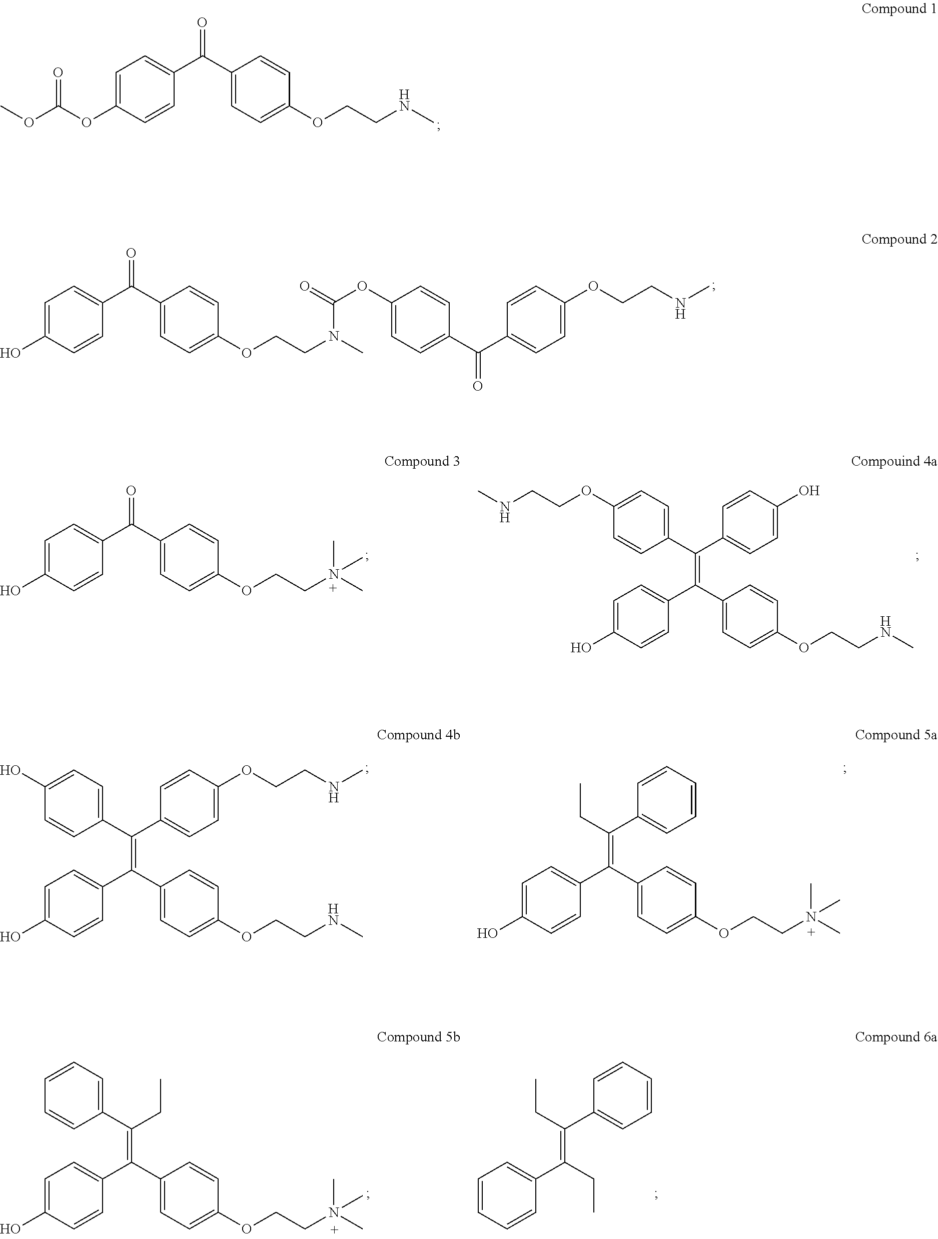

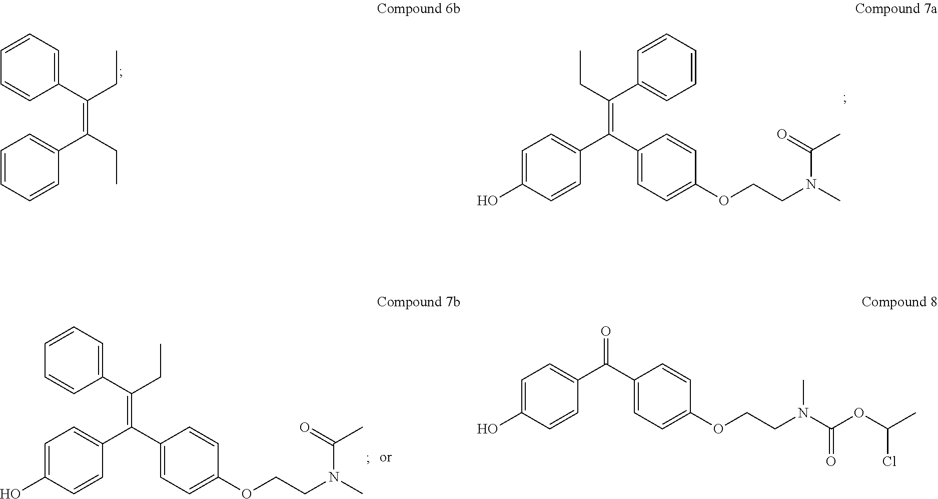

In some aspects, the endoxifen is not less than 90% (Z)-endoxifen:

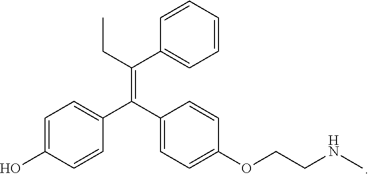 [SMILES: CC/C(=C(\c1ccc(O)cc1)c1ccc(OCCNC)cc1)c1ccccc1]

In some aspects, the endoxifen is between 50% and 60%, between 50% and 70%, between 50% and 75%, between 50% and 80%, between 50% and 85%, between 50% and 90%, between 50% and 95%, between 50% and 97.5%, between 50% and 99%, between 50% and 99.5%, between 60% and 70%, between 60% and 75%, between 60% and 80%, between 60% and 85%, between 60% and 90%, between 60% and 95%, between 60% and 97.5%, between 60% and 99%, between 60% and 99.5%, between 70% and 75%, between 70% and 80%, between 70% and 85%, between 70% and 90%, between 70% and 95%, between 70% and 97.5%, between 70% and 99%, between 70% and 99.5%, between 75% and 80%, between 75% and 85%, between 75% and 90%, between 75% and 95%, between 75% and 97.5%, between 75% and 99%, between 75% and 99.5%, between 80% and 85%, between 80% and 90%, between 80% and 95%, between 80% and 97.5%, between 80% and 99%, between 80% and 99.5%, between 85% and 90%, between 85% and 95%, between 85% and 97.5%, between 85% and 99%, between 85% and 99.5%, between 90% and 95%, between 90% and 97.5%, between 90% and 99%, between 90% and 99.5%, between 95% and 97.5%, between 95% and 99%, between 95% and 99.5%, between 97.5% and 99.5%, not less than 95%, not less than 96%, not less than 97%, not less than 98%, not less than 99%, or not less than 99.5% (Z)-endoxifen:

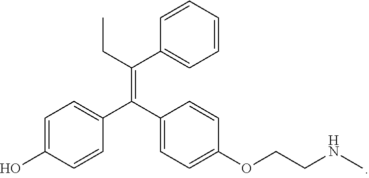 [SMILES: CC/C(=C(\c1ccc(O)cc1)c1ccc(OCCNC)cc1)c1ccccc1]

In some aspects, the composition comprises not less than 0.01% and not more than 0.1%, not less than 0.1% and not more than 1%, not less than 0.25% and not more than 1%, not less than 0.5% and not more than 1.5%, not less than 0.5% and not more than 2.5%, not less than 0.5% and not more than 5%, not less than 1% and not more than 2%, not less than 1% and not more than 2.25%, not less than 1% and not more than 2.5%, not less than 1% and not more than 3.75%, not less than 1% and not more than 4%, not less than 1% and not more than 4.25%, not less than 1% and not more than 4.5%, not less than 1% and not more than 4.75%, not less than 1% and not more than 5%, not less than 1% and not more than 5.5%, not less than 1% and not more than 6%, not less than 1% and not more than 6.5%, not less than 1% and not more than 7%, or not less than 1% and not more than 7.5% w/w of the further component.

In some aspects, the composition comprises not less than 0.010% and not more than 0.10%, not less than 0.1% and not more than 1%, not less than 0.25% and not more than 1%, or not less than 0.5% and not more than 1.5% w/w of Compound 1. In some aspects, the composition comprises not less than 0.01% and not more than 0.1%, not less than 0.1% and not more than 1%, not less than 0.25% and not more than 1%, or not less than 0.5% and not more than 1.5% w/w of Compound 2. In some aspects, the composition comprises not less than 0.01% and not more than 0.1%, not less than 0.1% and not more than 1%, not less than 0.25% and not more than 1%, or not less than 0.5% and not more than 1.5% w/w of Compound 3. In some aspects, the composition comprises not less than 0.01% and not more than 0.1%, not less than 0.1% and not more than 1%, not less than 0.25% and not more than 1%, or not less than 0.5% and not more than 1.5% w/w of Compounds 4a and 4b. In some aspects, the composition comprises not less than 0.01% and not more than 0.1%, not less than 0.1% and not more than 1%, not less than 0.25% and not more than 1%, or not less than 0.5% and not more than 1.5% w/w of Compounds 5a and 5b. In some aspects, the composition comprises not less than 0.01% and not more than 0.1%, not less than 0.1% and not more than 1%, not less than 0.25% and not more than 1%, or not less than 0.5% and not more than 1.5% w/w of Compounds 6a and 6b. In some aspects, the composition comprises not less than 0.01% and not more than 0.10%, not less than 0.1% and not more than 1%, not less than 0.25% and not more than 1%, or not less than 0.5% and not more than 1.5% w/w of Compounds 7a and 7b.

In some aspects, the composition has aromatase inhibition activity. In some aspects, the composition has an affinity to an estrogen receptor.

In some aspects, the pharmaceutically acceptable salt of the endoxifen is selected from the group consisting of an: arecoline, besylate, bicarbonate, bitartarate, butylbromide, citrate, camysylate, gluconate, glutamate, glycollylarsanilate, hexylresorcinate, hydrabamine, hydrobromide, hydrochloride, hydroxynapthanoate, isethionate, malate, mandelate, mesylate, methylbromide, methylnitrate, methylsulfate, mucate, napsylate, nitrate, pamaoate (Embonate), pantothenate, phosphate/diphosphate, polygalacuronate, salicylate, stearate, sulfate, tannate, Teoclate, triethiodide, benzathine, clemizole, chloroprocaine, choline, diethylamine, diethanolamine, ethylenediamine, meglumine, piperazine, procaine, aluminum, barium, bismuth, lithium, magnesium, potassium, and zinc salt. In some aspects, the pharmaceutically acceptable salt of the endoxifen is endoxifen gluconate. In some aspects, the pharmaceutically acceptable salt of the endoxifen is endoxifen citrate. In some aspects, the pharmaceutically acceptable salt of the endoxifen is endoxifen hydrochloride.

In some aspects, the composition is formulated as a suspension, a tablet, a capsule, a caplet, a liquid, or a gel. In some aspects, the composition comprises an alcohol, a plant oil, a mineral oil, a glycol, an agar, or a mixture thereof. In some aspects, the composition is formulated as an enteric formulation. In some aspects, the composition comprises hydroxypropyl methyl cellulose. In some aspects, the composition is formulated as a delayed release composition.

In some aspects, the composition comprises from 0.01 mg to 200 mg, from 0.1 mg to 100 mg, from 0.1 mg to 50 mg, from 0.1 mg to 20 mg, or from 1 mg to 20 mg of the endoxifen. In some aspects, the composition comprises a 0.05 mg/kg unit dose, a 0.1 mg/kg unit dose, a 0.5 mg/kg unit dose, a 1 mg/kg unit dose, or a 1.5 mg/kg unit dose of the endoxifen.

In certain aspects, the composition comprises less than 2.5%, less than 2%, less than 1.5%, less than 1%, less than 0.75%, less than 0.5%, less than 0.25%, less than 0.2%, less than 0.15%, less than 0.1%, or less than 0.05% water content. In additional aspects, the composition comprises less than 1%, less than 0.8%, less than 0.6%, less than 0.4%, less than 0.2%, less than 0.1%, or less than 0.05% sulphated ash. In further aspects, the composition comprises less than 6000 ppm methanol, less than 5000 ppm methanol, less than 4000 ppm methanol, less than 3000 ppm methanol, less than 2000 ppm methanol, less than 1000 ppm methanol, less than 500 ppm methanol, less than 250 ppm methanol, less than 200 ppm methanol, less than 150 ppm methanol, less than 100 ppm methanol, less than 75 ppm methanol, less than 50 ppm methanol, less than 25 ppm methanol, or less than 10 ppm methanol. In another aspect, the composition comprises less than 9000 ppm ethanol, less than 6000 ppm ethanol, less than 5000 ppm ethanol, less than 4000 ppm ethanol, less than 3000 ppm ethanol, less than 2000 ppm ethanol, less than 1000 ppm ethanol, less than 500 ppm ethanol, less than 250 ppm ethanol, less than 200 ppm ethanol, less than 150 ppm ethanol, less than 100 ppm ethanol, less than 75 ppm ethanol, less than 50 ppm ethanol, less than 25 ppm ethanol, or less than 10 ppm ethanol. In a particular aspect, the composition comprises less than 9000 ppm acetone, less than 6000 ppm acetone, less than 5000 ppm acetone, less than 4000 ppm acetone, less than 3000 ppm acetone, less than 2000 ppm acetone, less than 1000 ppm acetone, less than 500 ppm acetone, less than 250 ppm acetone, less than 200 ppm acetone, less than 150 ppm acetone, less than 100 ppm acetone, less than 75 ppm acetone, less than 50 ppm acetone, less than 25 ppm acetone, or less than 10 ppm acetone. In a specific aspect, the composition comprises less than 9000 ppm isopropyl alcohol, less than 6000 ppm isopropyl alcohol, less than 5000 ppm isopropyl alcohol, less than 4000 ppm isopropyl alcohol, less than 3000 ppm isopropyl alcohol, less than 2000 ppm isopropyl alcohol, less than 1000 ppm isopropyl alcohol, less than 500 ppm isopropyl alcohol, less than 250 ppm isopropyl alcohol, less than 200 ppm isopropyl alcohol, less than 150 ppm isopropyl alcohol, less than 100 ppm isopropyl alcohol, less than 75 ppm isopropyl alcohol, less than 50 ppm isopropyl alcohol, less than 25 ppm isopropyl alcohol, or less than 10 ppm isopropyl alcohol. In a further aspect, the composition comprises less than 1000 ppm acetonitrile, less than 500 ppm acetonitrile, less than 250 ppm acetonitrile, less than 200 ppm acetonitrile, less than 150 ppm acetonitrile, less than 100 ppm acetonitrile, less than 75 ppm acetonitrile, less than 50 ppm acetonitrile, less than 25 ppm acetonitrile, less than 10 ppm acetonitrile, or less than 5 ppm acetonitrile. In another aspect, the composition comprises less than 9000 ppm ethyl acetate, less than 6000 ppm ethyl acetate, less than 5000 ppm ethyl acetate, less than 4000 ppm ethyl acetate, less than 3000 ppm ethyl acetate, less than 2000 ppm ethyl acetate, less than 1000 ppm ethyl acetate, less than 500 ppm ethyl acetate, less than 250 ppm ethyl acetate, less than 200 ppm ethyl acetate, less than 150 ppm ethyl acetate, less than 100 ppm ethyl acetate, less than 75 ppm ethyl acetate, less than 50 ppm ethyl acetate, less than 25 ppm ethyl acetate, or less than 10 ppm ethyl acetate. In an additional aspect, the composition comprises less than 1500 ppm tetrahydrofuran, 1000 ppm tetrahydrofuran, less than 500 ppm tetrahydrofuran, less than 250 ppm tetrahydrofuran, less than 200 ppm tetrahydrofuran, less than 150 ppm tetrahydrofuran, less than 100 ppm tetrahydrofuran, less than 75 ppm tetrahydrofuran, less than 50 ppm tetrahydrofuran, less than 25 ppm tetrahydrofuran, less than 10 ppm tetrahydrofuran, or less than 5 ppm tetrahydrofuran. In a certain aspect, the composition comprises less than 1500 ppm 2-methyltetrahydrofuran, 1000 ppm 2-methyltetrahydrofuran, less than 500 ppm 2-methyltetrahydrofuran, less than 250 ppm 2-methyltetrahydrofuran, less than 200 ppm 2-methyltetrahydrofuran, less than 150 ppm 2-methyltetrahydrofuran, less than 100 ppm 2-methyltetrahydrofuran, less than 75 ppm 2-methyltetrahydrofuran, less than 50 ppm 2-methyltetrahydrofuran, less than 25 ppm 2-methyltetrahydrofuran, less than 10 ppm 2-methyltetrahydrofuran, or less than 5 ppm 2-methyltetrahydrofuran. In some aspects, the composition comprises less than 250 ppm benzene, less than 200 ppm benzene, less than 150 ppm benzene, less than 100 ppm benzene, less than 75 ppm benzene, less than 50 ppm benzene, less than 25 ppm benzene, less than 10 ppm benzene, less than 5 ppm benzene, less than 2.5 ppm benzene, less than 2 ppm benzene, less than 1.5 ppm benzene, less than 1 ppm benzene, or less than 0.5 ppm benzene. In further aspects, the composition comprises less than 250 ppm mesityl oxide, less than 200 ppm mesityl oxide, less than 150 ppm mesityl oxide, less than 100 ppm mesityl oxide, less than 75 ppm mesityl oxide, less than 50 ppm mesityl oxide, less than 25 ppm mesityl oxide, less than 10 ppm mesityl oxide, less than 5 ppm mesityl oxide, less than 2.5 ppm mesityl oxide, less than 2 ppm mesityl oxide, less than 1.5 ppm mesityl oxide, less than 1 ppm mesityl oxide, or less than 0.5 ppm mesityl oxide. In additional aspects, the composition comprises less than 500 ppm zinc, less than 250 ppm zinc, less than 200 ppm zinc, less than 150 ppm zinc, less than 100 ppm zinc, less than 75 ppm zinc, less than 50 ppm zinc, less than 25 ppm zinc, less than 10 ppm zinc, less than 5 ppm zinc, less than 2.5 ppm zinc, less than 2 ppm zinc, less than 1.5 ppm zinc, less than 1 ppm zinc. In specific aspects, the composition comprises less than 500 ppm titanium, less than 250 ppm titanium, less than 200 ppm titanium, less than 150 ppm titanium, less than 100 ppm titanium, less than 75 ppm titanium, less than 50 ppm titanium, less than 25 ppm titanium, less than 10 ppm titanium, less than 5 ppm titanium, less than 2.5 ppm titanium, less than 2 ppm benzene, less than 1.5 ppm titanium, less than 1 ppm titanium.

In various aspects, the present disclosure provides a pharmaceutical composition comprising the composition as described herein and a pharmaceutically acceptable excipient or diluent.

In some aspects, the composition as described herein is present in between 40-60%, 50-70%, 60-80%, 70-90%, 80-90%, 85-95%, 90-95%, or 95-99% w/w.

In various aspects, the present disclosure provides a method comprising administering the composition as described herein or the pharmaceutical composition as described herein to a subject.

In some aspects, the method further comprises treating a condition in a subject in need thereof. In some aspects, the subject has or is at risk of having a hormone-dependent breast disorder, a hormone-dependent reproductive tract disorder, or both. In some aspects, the hormone-dependent breast disorder or the hormone-dependent reproductive tract disorder is a benign breast disorder, hyperplasia, atypia, atypical ductal hyperplasia, atypical lobular hyperplasia, increased breast density, gynecomastia, DCIS, LCIS, breast cancer, precocious puberty, McCune-Albright Syndrome, endometrial cancer, ovarian cancer, uterine cancer, cervical cancer, vaginal cancer, or vulvar cancer. In some aspects, the subject has prostate cancer and wherein the subject further has or is at risk of having gynecomastia. In some aspects, the subject has tamoxifen-refractory or tamoxifen resistant hormone-dependent breast disorder or hormone-dependent reproductive tract disorder. In some aspects, the subject is or will be treated with an SSRI drug selected from the group consisting of citalopram, escitalopram, fluoxetine, paroxetine, sertraline, and vilazodone. In some aspects, the subject is administered 1 mg, 2 mg, 4 mg, 6 mg, 10 mg, or 20 mg of the endoxifen daily.

In various aspects, the present disclosure provides a method of producing the composition as described herein, the method comprising purifying a composition of Compound S3 to comprise >90% of Compound E1.

In some aspects, the method is performed in an organic solvent. In some aspects, the organic solvent comprises an aprotic solvent. In some aspects, the organic solvent comprises ethyl acetate. In some aspects, the method further comprises the addition of an acid. In some aspects, the acid comprises between about 2 to 12 N of the acid. In some aspects, the acid comprises HCl. In some aspects, the method comprises maintaining the coupling at a temperature of between 0 to 20° C., between −5 to 10° C., or between −10 to 10° C. during the addition of the acid.

In some aspects, the method further comprises heating the coupling to between 50 to 60° C., between 55 to 65° C., or between 60 to 70° C. In some aspects, the method further comprises stirring the solution for between 5 to 15 hours, between 8 to 12 hours, or between 9 to 11 hours. In some aspects, the method further comprises washing, purifying, or separating Compound S3 using a base, an aqueous solution, an alcohol, an organic solvent, or a combination thereof. In some aspects, the base is sodium hydroxide. In some aspects, the aqueous solution is a sodium chloride solution. In some aspects, the sodium chloride solution is between 5 to 25% NaCl, between 10 to 30% NaCl, or between 15 to 25% NaCl. In some aspects, the alcohol is ethanol, 2-propanol, or isopropyl alcohol. In some aspects, the organic solvent is ethyl acetate or acetone. In some aspects, the method further comprises adding a base to the purification increasing the pH to a basic pH. In some aspects, the base comprises about 2N to 12N of the base. In some aspects, the base is sodium hydroxide. In some aspects, the basic pH is between about 10 to 12. In some aspects, the purification comprises separation, filtration, distillation, isolation, drying, and slurry formation. In some aspects, the slurry formation uses an organic solvent. In some aspects of the slurry formation, the organic solvent is ethyl acetate.

In some aspects, the method further comprises: producing Compound S3 by coupling Compound S2 with propiophenone. In some aspects, the coupling comprises an olefination reaction. In some aspects, the coupling comprises a McMurry reaction. In some aspects, the method comprises activating Compound S2 and propiophenone with a metal catalyst. In some aspects, the method comprising generating the metal catalyst by combining a metal salt with a reducing agent. In some aspects, the metal catalyst comprises titanium, zirconium, vanadium, niobium, molybdenum, tungsten, aluminum, or zinc. In some aspects, the metal catalyst comprises titanium. In some aspects, the metal salt comprises a titanium halide. In some aspects, the titanium halide comprises TiCl₃ or TiCl₄. In certain aspects, the method comprises between 1 and 1.5, 1 and 2, 1 and 2.5, 1 and 3, 1 and 4, 1 and 5, 1 and 6, 1.5 and 2, 1.5 and 2.5, 1.5 and 3, 1.5 and 4, 1.5 and 5, 1.5 and 6, 2 and 2.5, 2 and 3, 2 and 4, 2 and 5, 2 and 6, 2.5 and 3, 2.5 and 4, 2.5 and 5, 2.5 and 6, 3 and 4, 3 and 5, 3 and 6, 4 and 5, 4 and 6, or 5 and 6 equivalents of the metal catalyst relative to the Compound S2.

In some aspects, the reducing agent comprises zinc, zirconium, vanadium, niobium, molybdenum, tungsten, aluminum, magnesium, potassium, zinc-copper couple, alkali and alkali earth metals, butylium, lithium, or lithium aluminum hydride. In some aspects, the zinc comprises zinc powder. In particular aspects, the method comprises between 2 and 3, 2 and 4, 2 and 5, 2 and 6, 2 and 8, 2 and 10, 3 and 4, 3 and 5, 3 and 6, 3 and 8, 3 and 10, 4 and 5, 4 and 6, 4 and 8, 4 and 10, 5 and 6, 5 and 8, 5 and 10, 6 and 8, 6 and 10, or 8 and 10 equivalents of the reducing agent relative to the Compound S2.

In some aspects, the method further comprises a first solution made by combining the reducing agent and an organic solvent under an inert atmosphere at a temperature between 15° to 30° C. In some aspects, the organic solvent is THF. In some aspects, the inert atmosphere is a N₂ atmosphere. In some aspects, the method further comprises cooling the first solution to a temperature between −10° to 5° C. and adding the metal catalyst. In some aspects, the method further comprises maintaining the first solution at a temperature less than 20° C. during the addition of the metal catalyst. In some aspects, the method further comprises heating the first solution to a temperature between 50° to 70° C. for 1 hour, 1.5 hours, 2 hours, 2.5 hours, or 3 hours.

In some aspects, the method further comprises a second solution made by adding Compound S2 and propiophenone to an organic solvent. In some aspects, the organic solvent is THF. In some aspects, the method further comprises heating the second solution to between 50° to 70° C.

In some aspects, the method further comprises a third solution made by adding the second solution to the first solution over 1 hour, 1.5 hours, or 2 hours. In some aspects, the method further comprises stirring the third solution at between 50° to 70° C. for 7 hours, 8 hours, 9 hours, or 10 hours.

In some aspects, the method further comprises distilling, washing, filtering, separating, extracting, precipitating, collecting, or a combination thereof, Compound S3 with an organic solvent, an acid, or an aqueous solution. In some aspects, the organic solvent is MeTHF. In some aspects, the washing comprises an organic solvent, an acid, a base, a solvent mixture, or a combination thereof. In some aspects, the organic solvent is MeTHF, acetonitrile, acetone, or a combination thereof. In some aspects, the acid is between 2N to 12 N HCl. In some aspects, the aqueous solution is 30-35% HCl, between 10% to 60% NaCl, or between 10% to 60% K₂CO₃.

In some aspects, the method further comprises producing Compound S2 by demethylating Compound S1. In some aspects, Compound S1 is demethylated by a demethylating agent. In some aspects, the demethylating agent is 1-chloroethyl chloroformate. In some aspects, the demethylating comprises 1 to 1.5, 1 to 2, 1 to 2.5, 1 to 3, 1 to 4, 1 to 5, 1.5 to 2, 1.5 to 2.5, 1.5 to 3, 1.5 to 4, 1.5 to 5, 2 to 2.5, 2 to 3, 2 to 4, 2 to 5, 2.5 to 3, 2.5 to 4, 2.5 to 5, 3 to 4, 3 to 5, or 4 to 5 equivalents of the demethylating agent relative to the Compound S1. In some aspects, the demethylation comprises an organic solvent. In some aspects, the organic solvent comprises a solvent mixture. In some aspects, the solvent mixture comprises tetrahydrofuran and N-ethyldiisopropylamine. In some aspects, the organic solvent comprises a base. In certain aspects, the base is a tertiary amine. In particular aspects, the base is ehtyldiisopropylamine. In particular aspects, the demethylating comprises between 1 and 2, 1 and 3, 1 and 4, 1 and 5, 1 and 6, 1 and 8, 2 and 3, 2 and 4, 2 and 5, 2 and 6, 2 and 8, 3 and 4, 3 and 5, 3 and 6, 3 and 8, 4 and 5, 4 and 6, 4 and 8, 5 and 6, 5 and 8, or 6 and 8 equivalents of the base relative to the Compound S1. In some aspects, the method further comprises incubating the demethylation under an inert atmosphere. In some aspects, the inert atmosphere is a N₂ atmosphere. In some aspects, the method further comprises heating the demethylation to between 30 to 70° C., between 40 to 70° C., between 50 to 70° C., or between 55 to 65°. In some aspects, the method further comprises heating the demethylation to an elevated temperature for between 8 to 16 hours, between 10 to 14 hours, or between 9 to 11 hours. In some aspects, the elevated temperature is a boiling point of the organic solvent.

In some aspects, the method further comprises adding an alcohol to the demethylation. In some aspects, the alcohol is methanol. In some aspects, the method further comprises heating the demethylation to between 30 to 70° C., between 40 to 70° C., between 50 to 70° C., or between 55 to 65° C. and distilling the coupling to a reduced volume. In some aspects, the reduced volume to total volume ratio is 1:3, 1:5, or 1:10. In some aspects, the method further comprises adding an acid. In some aspects, the acid comprises 2N to 12N of the acid. In some aspects, the acid is hydrochloric acid.

In some aspects, the separating comprises phase separation. In some aspects, the separating comprises distillation. In particular aspects, the distillation comprises vacuum distillation. In further aspects, the distillation is performed at atmospheric pressure. In additional aspects, the distillation is performed at a temperature of between 5° C. and 10° C., 5° C. and 15° C., 5° C. and 20° C., 5° C. and 25° C., 5° C. and 30° C., 5° C. and 40° C., 5° C. and 50° C., 10° C. and 15° C., 10° C. and 20° C., 10° C. and 25° C., 10° C. and 30° C., 10° C. and 40° C., 10° C. and 50° C., 15° C. and 20° C., 15° C. and 25° C., 15° C. and 30° C., 15° C. and 40° C., 15° C. and 50° C., 20° C. and 25° C., 20° C. and 30° C., 20° C. and 35° C., 20° C. and 40° C., 20° C. and 50° C., 25° C. and 30° C., 25° C. and 40° C., 25° C. and 50° C., 30° C. and 40° C., 30° C. and 50° C., or 40° C. and 50° C.

In one aspect, the separating uses between 1 and 2, 1 and 3, 1 and 4, 1 and 5, 1 and 6, 1 and 8, 1 and 10, 1 and 12, 1.5 and 3, 1.5 and 4, 1.5 and 5, 1.5 and 6, 1.5 and 8, 1.5 and 10, 1.5 and 12, 2 and 3, 2 and 4, 2 and 5, 2 and 6, 2 and 8, 2 and 10, 2 and 12, 3 and 4, 3 and 5, 3 and 6, 3 and 8, 3 and 10, 3 and 12, 4 and 5, 4 and 6, 4 and 8, 4 and 10, 4 and 12, 5 and 6, 5 and 8, 5 and 10, 5 and 12, 6 and 8, 6 and 10, 6 and 12, 8 and 10, 8 and 12, or 10 and 12 equivalents of the acid (weight/weight). In another aspect, the separating uses between 3.2 and 4.8 equivalents of the acid (weight/weight). In a further aspect, the Compound S2 comprises less than 5%, less than 4%, less than 3%, less than 2.5%, less than 2%, less than 1.5%, less than 1%, less than 0.75%, less than 0.5%, less than 0.25%, or less than 0.1% Compound 8. In particular aspects, the Compound S2 exhibits less than 10%, less than 8%, less than 6%, less than 5%, less than 4%, less than 3%, less than 2%, less than 1.5%, or less than 1% weight loss upon drying. In further aspects, the Compound S2 comprises less than 5000 ppm, less than 4000 ppm, less than 3000 ppm, less than 2500 ppm, less than 2000 ppm, less than 1500 ppm, less than 1000 ppm, less than 500 ppm, less than 200 ppm, or less than 100 ppm DIPEA.

In some aspects, the method further comprises heating the demethylation to between 30 to 70° C., between 40 to 70° C., between 50 to 70° C., or between 55 to 65° C. for between 2 to 20 hours, between 6 to 18 hours, or between 8 to 16 hours.

In some aspects, the method further comprises distilling, washing, purifying, or separating Compound S2 using an acid, a base, an aqueous solution, an alcohol, or an organic solvent. In some aspects, the aqueous solution is water. In some aspects, the base comprises 2N to 12N of the base. In some aspects, the base is sodium hydroxide. In some aspects, the organic solvent is ethyl acetate. In some aspects, the acid comprises 2N to 12N of the acid. In some aspects, the acid is hydrochloric acid.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a UV chromatogram of Compound 1.

FIG. 2 illustrates a TIC chromatogram of Compound 1.

FIG. 3 illustrates a mass spectrum of Compound 1.

FIG. 4 illustrates a proton NMR spectrum of Compound 1.

FIG. 5 illustrates a carbon NMR spectrum of Compound 1.

FIG. 6 illustrates a proton NMR spectrum of Compound 2.

FIG. 7 illustrates a carbon NMR spectrum of Compound 2.

FIG. 8 illustrates a COSY NMR 2D spectrum of Compound 2.

FIG. 9 illustrates a UV chromatogram of Compound 2.

FIG. 10 illustrates a TIC chromatogram of Compound 2.

FIG. 11 illustrates a mass spectrum of Compound 2.

FIG. 12 illustrates a proton NMR spectrum of Compound 3.

FIG. 13 illustrates a UV chromatogram of Compound 3.

FIG. 14 illustrates a TIC chromatogram of Compound 3.

FIG. 15 illustrates a mass spectrum of Compound 3.

FIG. 16 illustrates a UV chromatogram of Compounds 4a and 4b.

FIG. 17 illustrates a TIC chromatogram of Compounds 4a and 4b.

FIG. 18 illustrates a mass spectrum of Compounds 4a and 4b.

FIG. 19 illustrates a UV chromatogram of Compound 7b.

FIG. 20 illustrates a TIC chromatogram of Compound 7b.

FIG. 21 illustrates a mass spectrum of Compound 7.

FIG. 22 illustrates a proton NMR spectrum of Compound 7b.

FIG. 23 illustrates a ROESY spectrum of Compound 7b.

FIG. 24 illustrates a proton NMR spectrum of Compound 7a.

FIG. 25 illustrates a ROESY spectrum of Compound 7a.

FIG. 26 illustrates a proton NMR spectrum of Compound 5b.

FIG. 27 illustrates a ROESY spectrum of Compound 5b.

FIG. 28 illustrates a proton NMR spectrum of Compound 5a.

FIG. 29 illustrates a ROESY spectrum of Compound 5a.

FIG. 30 illustrates a UV chromatogram of Compound 5b.

FIG. 31 illustrates a TIC chromatogram of Compound 5b.

FIG. 32 illustrates a mass spectrum of Compound 5b.

FIG. 33 illustrates an HPLC chromatogram of Compounds 6a and 6b and a reference standard of Compounds 6a and 6b.

FIG. 34 illustrates a contour plot of (Z)-endoxifen production as a function of temperature and maintenance time.

### DETAILED DESCRIPTION

Described herein are endoxifen compositions with novel compounds and methods of making thereof that may have anti-tumor activity and provide treatments for diseases, such as cancer. Also described are synthetic methods of making the pharmaceutical compounds described herein and methods of making the pharmaceutical compositions as described herein. The compounds or compositions described herein may exhibit aromatase inhibition activity or estrogen receptor activity. The compounds and compositions described herein may be useful in the treatment of a condition in a subject (e.g., the treatment of cancer in a subject).

### Endoxifen and Endoxifen-Related Compounds

The chemical structures of endoxifen and endoxifen-related compounds of the current disclosure are provided in TABLE 1. Some of the compounds listed in TABLE 1 may be used as substrates for endoxifen and (Z)-endoxifen synthesis (e.g., Compound S1, Compound S2, and Compound S3). Endoxifen may be present in the compositions of the current disclosure as a mix of (E)-endoxifen and (Z)-endoxifen (e.g., Compound S3). Endoxifen may be present in the compositions of the current disclosure as (Z)-endoxifen (Compound E1). Other endoxifen-related compounds may be present in the compositions of the disclosure (e.g., Compound 1, Compound 2, Compound 3, Compound 4a, Compound 4b, Compound 5a, Compound 5b, Compound 6a, Compound 6b, Compound 7a, Compound 7b, Compound 8, or a pharmaceutically acceptable salts, solvates, or tautomers thereof).

TABLE 1
Endoxifen and Endoxifen-Related Compounds of the Present Disclosure or
Pharmaceutically Acceptable Salts, Solvates, or Tautomers Thereof

| Compound | Structure |
|---|---|
| Compound S1 | 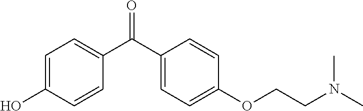 [SMILES: CN(C)CCOc1ccc(C(=O)c2ccc(O)cc2)cc1] |
| Compound S2 | 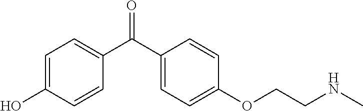 [SMILES: CNCCOc1ccc(C(=O)c2ccc(O)cc2)cc1] |
| Compound S3 Endoxifen | 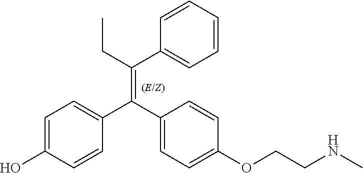 [SMILES: CC/C(=C(\c1ccc(O)cc1)c1ccc(OCCNC)cc1)c1ccccc1] |
| Compound E1 (Z)-endoxifen | 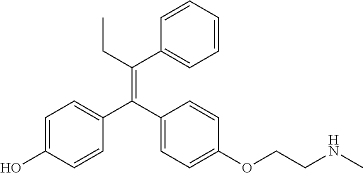 [SMILES: CC/C(=C(\c1ccc(O)cc1)c1ccc(OCCNC)cc1)c1ccccc1] |
| Compound 1 | 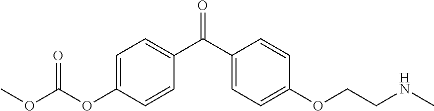 |
| Compound 2 | 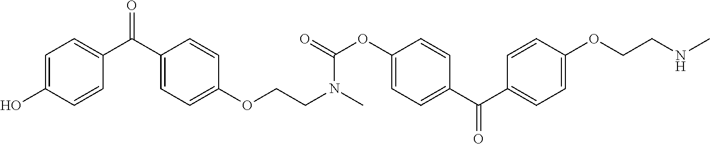 [SMILES: CNCCOc1ccc(C(=O)c2ccc(OC(=O)N(C)CCOc3ccc(C(=O)c4ccc(O)cc4)cc3)cc2)cc1] |
| Compound 3 | 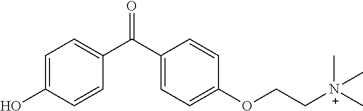 [SMILES: C[N+](C)(C)CCOc1ccc(C(=O)c2ccc(O)cc2)cc1] |
| Compound 4a | 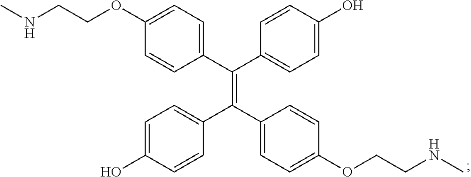 |
| Compound 4b | 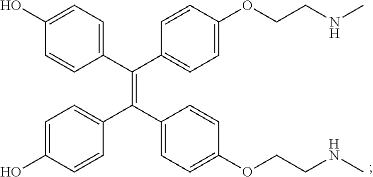 [SMILES: CNCCOc1ccc(/C(=C(/c2ccc(O)cc2)c2ccc(OCCNC)cc2)c2ccc(O)cc2)cc1] |
| Compound 5a | 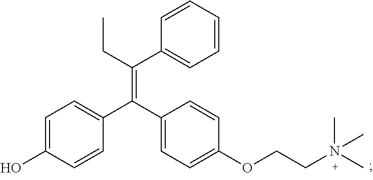 |
| Compound 5b | 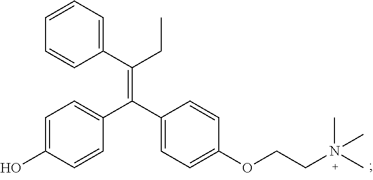 |
| Compound 6a | 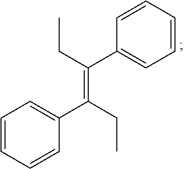 [SMILES: CC/C(=C(/CC)c1ccccc1)c1ccccc1] |
| Compound 6b | 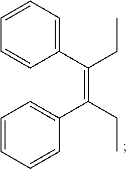 [SMILES: CC/C(=C(\CC)c1ccccc1)c1ccccc1] |
| Compound 7a | 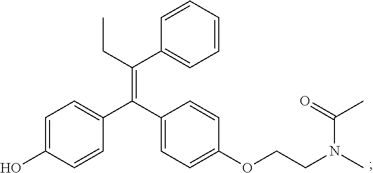 [SMILES: CC/C(=C(\c1ccc(O)cc1)c1ccc(OCCN(C)C(C)=O)cc1)c1ccccc1] |
| Compound 7b | 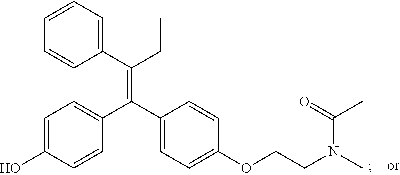 [SMILES: CC/C(=C(/c1ccc(O)cc1)c1ccc(OCCN(C)C(C)=O)cc1)c1ccccc1] |
| Compound 8 | 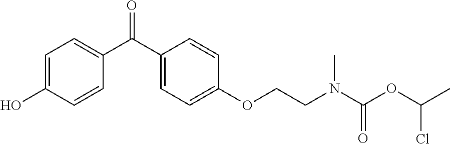 [SMILES: CC(Cl)OC(=O)N(C)CCOc1ccc(C(=O)c2ccc(O)cc2)cc1] |

The compounds of the present disclosure may have isomers (e.g., structural isomers, tautomers, stereoisomers, enantiomers, rotamers, topoisomers, or isotopomers). The compounds of the present disclosure may have (F) and (Z) isomers.

Compound 1, Compound 2, Compound 3, Compound 4a, Compound 4b, Compound 5a, Compound 5b, Compound 6a, Compound 6b, Compound 7a, Compound 7b, Compound 8, or a pharmaceutically acceptable salt, solvate, or tautomer thereof may be in a cationic or anionic form. For example, compounds may comprise a deprotonated hydroxyl or a quaternary amine resulting in an overall negative or positive charge, respectively. A “salt” of a compound described herein may be derived from inorganic or organic acids and bases. Suitable counter-anions include, arecoline, besylate, bicarbonate, bitartarate, butylbromide, citrate, camysylate, gluconate, glutamate, glycollylarsanilate, hexylresorcinate, hydrabamine, hydrobromide, hydrochloride, hydroxynapthanoate, isethionate, malate, mandelate, mesylate, methylbromide, methylnitrate, methylsulfate, mucate, napsylate, nitrate, pamaoate (Embonate), pantothenate, phosphate/diphosphate, polygalacuronate, salicylate, stearate, sulfate, tannate, teoclate, fatty acid anions, and triethiodide. Suitable counter-cations include benzathine, clemizole, chloroprocaine, choline, diethylamine, diethanolamine, ethylenediamine, meglumine, piperazine, procaine, aluminum, barium, bismuth, lithium, magnesium, potassium, and zinc. In some embodiments, an endoxifen salt may comprise arecoline, besylate, bicarbonate, bitartarate, butylbromide, citrate, camysylate, gluconate, glutamate, glycollylarsanilate, hexylresorcinate, hydrabamine, hydrobromide, hydrochloride, hydroxynapthanoate, isethionate, malate, mandelate, mesylate, methylbromide, methylnitrate, methylsulfate, mucate, napsylate, nitrate, pamaoate (Embonate), pantothenate, phosphate/diphosphate, polygalacuronate, salicylate, stearate, sulfate, tannate, Teoclate, triethiodide, benzathine, clemizole, chloroprocaine, choline, diethylamine, diethanolamine, ethylenediamine, meglumine, piperazine, procaine, aluminum, barium, bismuth, lithium, magnesium, potassium, or a zinc salt. In some embodiments, a salt of Compound 1, Compound 2, Compound 3, Compound 4a, Compound 4b, Compound 5a, Compound 5b, Compound 6a, Compound 6b, Compound 7a, Compound 7b, or Compound 8 may comprise arecoline, besylate, bicarbonate, bitartarate, butylbromide, citrate, camysylate, gluconate, glutamate, glycollylarsanilate, hexylresorcinate, hydrabamine, hydrobromide, hydrochloride, hydroxynapthanoate, isethionate, malate, mandelate, mesylate, methylbromide, methylnitrate, methylsulfate, mucate, napsylate, nitrate, pamaoate (Embonate), pantothenate, phosphate/diphosphate, polygalacuronate, salicylate, stearate, sulfate, tannate, Teoclate, triethiodide, benzathine, clemizole, chloroprocaine, choline, diethylamine, diethanolamine, ethylenediamine, meglumine, piperazine, procaine, aluminum, barium, bismuth, lithium, magnesium, potassium, or a zinc salt.

### Endoxifen Compositions

A mixture of (E)-endoxifen and (Z)-endoxifen, also referred to as (E/Z)-endoxifen or Compound S3 herein, can be represented by Formula (I):

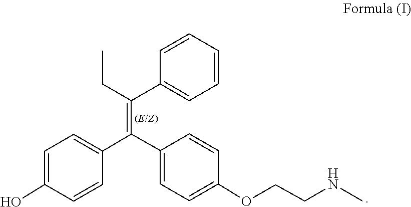 [SMILES: CC/C(=C(\c1ccc(O)cc1)c1ccc(OCCNC)cc1)c1ccccc1]

(Z)-endoxifen, also referred to as Compound E1 herein, can be represented by Compound E1:

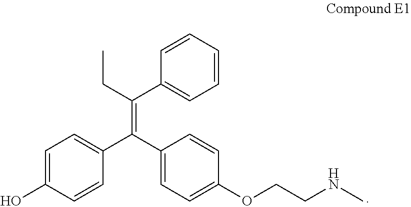 [SMILES: CC/C(=C(\c1ccc(O)cc1)c1ccc(OCCNC)cc1)c1ccccc1]

(E)-endoxifen can be represented by Compound E2:

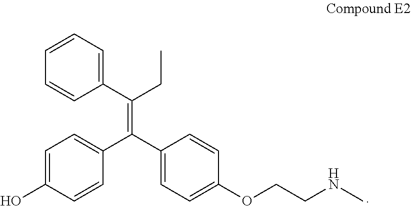 [SMILES: CC/C(=C(/c1ccc(O)cc1)c1ccc(OCCNC)cc1)c1ccccc1]

In some embodiments, the endoxifen compositions of the present disclosure may comprise (Z)-endoxifen as at least about 50%, at least about 60%, at least about 70%, at least about 80%, at least about 90%, at least about 91%, at least about 92%, at least about 93%, at least about 94%, at least about 95%, at least about 96%, at least about 97%, at least about 98%, at least about 99%, at least about 99.5%, at least about 99.99%, or about 100% of total endoxifen (e.g., (Z)-endoxifen and (E)-endoxifen), by weight, in the composition. In some embodiments, the composition comprises at least 90% of (Z)-endoxifen with respect to total endoxifen, by weight, in the composition. In some embodiments, the composition comprises at least 95% of (Z)-endoxifen with respect to total endoxifen, by weight, in the composition. In some embodiments, the composition comprises at least about 96%, at least about 97%, at least about 98%, at least about 99%, at least about 99.5%, or at least about 99.9% of (Z)-endoxifen with respect to total endoxifen, by weight, in the composition. In some embodiments, the endoxifen compositions of the present disclosure may comprise (E)-endoxifen as no more than about 10%, no more than about 8%, no more than about 6%, no more than about 7%, no more than about 8%, no more than about 9%, no more than about 2%, or no more than about 1% of total endoxifen (e.g., (Z)-endoxifen and (E)-endoxifen), by weight, in the composition.

In some embodiments, the endoxifen compositions of the present disclosure may comprise a ratio of (Z)-endoxifen to (E)-endoxifen (Z:E ratio) that is at least about 50:50, at least about 60:40, at least about 64:36, at least about 70:30, at least about 80:30, at least about 82:18, at least about 85:15, at least about 90:10, at least about 94:6, at least about 95:5, at least about 96:4, at least about 97:3, at least about 98:2, at least about 99:1, or about 100:0. In some embodiments, the endoxifen compositions of the present disclosure may comprise a ratio of (Z)-endoxifen to (E)-endoxifen (Z:E ratio) that is from about 50:50 to about 100:0, from about 60:40 to about 100:0, from about 64:36 to about 100:0, from about 70:30 to about 100:0, from about 80:30 to about 100:0, from about 82:18 to about 100:0, from about 85:15 to about 100:0, from about 90:10 to about 100:0, from about 94:6 to about 100:0, from about 95:5 to about 100:0, from about 96:4 to about 100:0, from about 97:3 to about 100:0, from about 98:2 to about 100:0, or from about 99:1 to about 100:0.

A composition comprising (Z)-endoxifen may comprise at least about 4% (Z)-endoxifen, by weight, relative to total fill weight of the composition (e.g., excluding, if present, a capsule or coating encapsulating the composition). A composition comprising (Z)-endoxifen may comprise at least about 10% (Z)-endoxifen, by weight, relative to total fill weight of the composition. In some embodiments, the composition comprising (Z)-endoxifen may comprise at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, or at least about 20% (Z)-endoxifen, by weight, relative to total fill weight of the composition. In some embodiments, the composition comprising (Z)-endoxifen may comprise from about 4% to about 50%, from about 5% to about 50%, from about 7% to about 50%, from about 10% to about 50%, from about 12% to about 50%, from about 15% to about 50%, from about 4% to about 40%, from about 5% to about 40%, from about 7% to about 40%, from about 10% to about 40%, from about 12% to about 40%, from about 15% to about 40%, from about 4% to about 30%, from about 5% to about 30%, from about 7% to about 30%, from about 10% to about 30%, from about 12% to about 30%, from about 15% to about 30%, from about 4% to about 20%, from about 5% to about 20%, from about 7% to about 20%, from about 10% to about 20%, from about 12% to about 20%, or from about 15% to about 20% (Z)-endoxifen, by weight, relative to total fill weight of the composition.

A composition of the present disclosure (e.g., an enteric-resistant delayed release high dose endoxifen formulation) may comprise an amount of (Z)-endoxifen formulated as a dosage form, such as a tablet or capsule. In some embodiments, a single high dose endoxifen dosage form (e.g., a single capsule or a single tablet) may comprise about 10 mg, about 12 mg, about 15 mg, about 16 mg, about 20 mg, about 22 mg, about 24 mg, about 25 mg, about 28 mg, about 30 mg, about 32 mg, about 35 mg, about 36 mg, about 38 mg, about 40 mg, about 42 mg, about 45 mg, about 50 mg, about 60 mg, about 70 mg, about 80 mg, about 90 mg, or about 100 mg of (Z)-endoxifen. In some embodiments, a single high dose endoxifen dosage form may comprise from about 10 mg to about 100 mg, from about 10 mg to about 80 mg, from about 10 mg to about 50 mg, from about 20 mg to about 50 mg, from about 30 mg to about 50 mg, or from about 10 mg to about 30 mg of (Z)-endoxifen.

Endoxifen, also referred to as 4-hydroxy-N-desmethyl-tamoxifen, may include a polymorphic, salt, free base, co-crystal, or solvate form of endoxifen. Examples of salts of (Z)-endoxifen suitable for the endoxifen compositions of the present disclosure (e.g., enteric-resistant delayed release high dose endoxifen formulations) include pharmacologically acceptable salts such as salts with inorganic acids, salts with organic acids, salts with amino acids and the like. Examples of (Z)-endoxifen salts with inorganic acids include salts with hydrochloric acid, hydrobromic acid, nitric acid, sulfuric acid, phosphoric acid, and the like. Examples of salts with organic acids include salts with formic acid, acetic acid, trifluoroacetic acid, fumaric acid, oxalic acid, tartaric acid, maleic acid, methanesulfonic acid, benzenesulfonic acid, p-toluenesulfonic acid, and the like. Examples of the salts with acidic amino acids include salts with aspartic acid, glutamic acid, citric acid, and the like.

Examples of anion salts of (Z)-endoxifen include arecoline, besylate, bicarbonate, bitartarate, butylbromide, citrate, camysylate, gluconate, glutamate, glycollylarsanilate, hexylresorcinate, hydrabamine, hydrobromide, hydrochloride, hydroxynapthanoate, isethionate, malate, mandelate, mesylate, methylbromide, methylbromide, methylnitrate, methylsulfate, mucate, napsylate, nitrate, pamaoate (Embonate), pantothenate, phosphate/diphosphate, polygalacuronate, salicylate, stearate, sulfate, tannate, Teoclate, and triethiodide. Examples of cation salts of (Z)-endoxifen selected from the group consisting of benzathine, clemizole, chloroprocaine, choline, diethylamine, diethanolamine, ethylenediamine, meglumine, piperazine, procaine, aluminum, barium, bismuth, lithium, magnesium, potassium, and zinc, and the like. In some embodiments, the present disclosure provides that embodiments include salts made with acids that are not pharmaceutically acceptable.

In some embodiments, an endoxifen composition of the present disclosure comprises salts of (Z)-endoxifen selected from the group consisting of acetate, arecoline, benzathine, benzoic, besylate, benzosulfonate, bicarbonate, bitartarate, butylbromide, citrate, camysylate, clemizole, chloroprocaine, choline, diethylamine, diethanolamine, ethylenediamine, formate, fumarate, glucolate, gluconate, glutamate, glycollylarsanilate, hexylresorcinate, hydrabamine, hydrobromide, hydrochloride, hydroxynapthanoate, isethionate, malate, maleate, mandelate, meglumine, mesylate, methylbromide, methylbromide, methylnitrate, methylsulfate, methanesulfonate, mucate, napsylate, nitric, nitrate, oxalate, pamaoate (Embonate), pantothenate, perchloric, phosphate, diphosphate, piperazine, procaine, polygalacuronate, p-toluenesulfonate, salicylate, stearate, succinate, sulfate, sulfonate, sulfuric, tannate, tartarate, teoclate, triethiodide, trifluoroacetate, aluminum, barium, bismuth, lithium, magnesium, potassium, and zinc aluminum, barium, bismuth, lithium, magnesium, potassium, and zinc, or any combination thereof. In some embodiments, the salt is (Z)-endoxifen gluconate. Endoxifen gluconate can be selected from the group consisting of (Z)-endoxifen D-gluconate, (Z)-endoxifen L-gluconate, or a combination thereof.

While the content of (Z)-endoxifen or a polymorph or a salt thereof in the endoxifen compositions of the present disclosure varies depending on the dosage form of the composition, target disease, severity of disease, and the like, it is an amount generally corresponding or equivalent to from about 0.01 mg to about 200 mg of (Z)-endoxifen. One of skill in the art will recognize that when a composition includes salts of (Z)-endoxifen, the endoxifen salt may be in an equivalent amount on the basis of (Z)-endoxifen to be released.

In some embodiments, the endoxifen may comprise one or more polymorphic forms, such as Form I, of endoxifen. A polymorphic form may be distinguished by its x-ray powder diffraction pattern. In some embodiments, a method of treating a cancer may comprise administering a pharmaceutical composition comprising endoxifen predominantly as polymorph Form I. In some embodiments, polymorphic Form I is characterized by an x-ray powder diffraction pattern comprising major peaks at 16.8±0.3°, 17.1±0.3° and 21.8±0.3° two theta. The crystalline form of Form I of the compound of endoxifen can be characterized by an XRPD pattern comprising major peaks at 16.8±0.3°, 17.1±0.3° and 21.8±0.3° two theta. In some cases, the XRPD pattern further comprises at least one peak, or at least two peaks, at least three peaks selected from 16.0±0.3°, 18.8±0.3° and 26.5±0.3° two theta. In some cases, the XRPD pattern further comprises at least one peak, or at least two peaks, at least three peaks selected from 12.4±0.3°, 28.0±0.3° and 29.0±0.3° two theta. In some embodiments, polymorphic Form I of endoxifen exhibits an x-ray powder diffraction (XRPD) pattern substantially as shown in WO2023/137044.

The endoxifen in a composition of the present disclosure may be present in one or more polymorphic forms (e.g., Form I). For example, a composition of the present disclosure may comprise at least about 90% of total endoxifen, by weight, as polymorph Form I. In another example, a composition of the present disclosure may comprise at least about 95% of total endoxifen, by weight, as polymorph Form I. When a particular percentage by weight of endoxifen is a single polymorphic form (e.g., Form I), the remainder of endoxifen in the composition may be some combination of amorphous endoxifen or one or more polymorphic forms of endoxifen excluding the single polymorphic form. In some embodiments, the endoxifen composition comprises (Z)-endoxifen predominantly as polymorph Form I.

### High Dose Formulations

High dose endoxifen formulations, such as enteric-resistant delayed release high dose endoxifen compositions, may present challenges due to the relatively high percent active pharmaceutical ingredient (% API) present in the formulation. Endoxifen formulations with high percent endoxifen relative to total composition fill weight (e.g., at least about 4% endoxifen, at least about 10% endoxifen, at least about 15% endoxifen, or from about 15% to about 20% endoxifen) may exhibit increased reactivity with an encapsulating layer (e.g., a coating or a capsule) compared to lower dose formulations (e.g., less than about 4% endoxifen). For example, a high dose endoxifen composition comprising at least about 4% endoxifen, by weight, relative to total fill weight may exhibit noticeable crosslinking with the surrounding capsule. Such reactivity (e.g., crosslinking) may negatively impact the bioavailability of endoxifen upon administration of the composition. For example, crosslinking between endoxifen and an enteric-resistant delayed release capsule encapsulating the endoxifen may inhibit dissolution of the capsule upon oral administration, preventing release of endoxifen. Reactivity of high dose endoxifen compositions may be mitigated by pharmaceutical excipients added to the endoxifen composition.

A high dose endoxifen composition of the present disclosure (e.g., an enteric-resistant delayed release high dose endoxifen formulation) may comprise an amount of (Z)-endoxifen formulated as a dosage form, such as a tablet or capsule. In some embodiments, a single high dose endoxifen dosage form (e.g., a single capsule or a single tablet) may comprise about 10 mg, about 12 mg, about 15 mg, about 16 mg, about 20 mg, about 22 mg, about 24 mg, about 25 mg, about 28 mg, about 30 mg, about 32 mg, about 35 mg, about 36 mg, about 38 mg, about 40 mg, about 42 mg, about 45 mg, about 50 mg, about 60 mg, about 70 mg, about 80 mg, about 90 mg, or about 100 mg of (Z)-endoxifen. In some embodiments, a single high dose endoxifen dosage form may comprise from about 10 mg to about 100 mg, from about 10 mg to about 80 mg, from about 10 mg to about 50 mg, from about 20 mg to about 50 mg, from about 30 mg to about 50 mg, or from about 10 mg to about 30 mg of (Z)-endoxifen.

In some embodiments, a high dose endoxifen composition may comprise from about 4% to about 40%, from about 5% to about 40%, from about 8% to about 40%, from about 10% to about 40%, from about 12% to about 40%, from about 13% to about 40%, from about 14% to about 40%, from about 15% to about 40%, from about 16% to about 40%, from about 17% to about 40%, from about 18% to about 40%, from about 4% to about 30%, from about 5% to about 30%, from about 8% to about 30%, from about 10% to about 30%, from about 12% to about 30%, from about 13% to about 30%, from about 14% to about 30%, from about 15% to about 30%, from about 16% to about 30%, from about 17% to about 30%, from about 18% to about 30%, from about 4% to about 25%, from about 5% to about 25%, from about 8% to about 25%, from about 10% to about 25%, from about 12% to about 25%, from about 13% to about 25%, from about 14% to about 25%, from about 15% to about 25%, from about 16% to about 25%, from about 17% to about 25%, from about 18% to about 25%, from about 4% to about 22%, from about 5% to about 22%, from about 8% to about 22%, from about 10% to about 22%, from about 12% to about 22%, from about 13% to about 22%, from about 14% to about 22%, from about 15% to about 22%, from about 16% to about 22%, from about 17% to about 22%, from about 18% to about 22%, from about 4% to about 20%, from about 5% to about 20%, from about 8% to about 20%, from about 10% to about 20%, from about 12% to about 20%, from about 13% to about 20%, from about 14% to about 20%, from about 15% to about 20%, from about 16% to about 20%, from about 17% to about 20%, from about 18% to about 20% (Z)-endoxifen, by weight, of the total fill weight of the composition,

A disintegrant may be included in a high dose endoxifen composition of the present disclosure to inhibit crosslinking between endoxifen and an encapsulating layer (e.g., capsule). In some embodiments, a disintegrant may comprise croscarmellose sodium, agar, alginic acid, calcium carbonate, microcrystalline cellulose, crospovidone, polacrilin potassium, sodium starch glycolate, potato or tapioca starch, pre-gelatinized starch, other starches, clays, other algins, other celluloses, gums, or a combination thereof. For example, croscarmellose sodium may be included in a high dose endoxifen composition to inhibit crosslinking between endoxifen and an enteric-resistant delayed release capsule encapsulating the endoxifen composition.

The disintegrant (e.g., croscarmellose sodium) may be included in a high dose endoxifen composition of the present disclosure at an amount of at least about 0.1%, at least about 0.5%, at least about 0.8%, at least about 1.0%, at least about 1.2%, at least about 1.4%, at least about 1.5%, at least about 1.6%, at least about 1.8%, at least about 2.0%, at least about 2.2%, at least about 2.4%, at least about 2.5%, at least about 2.6%, at least about 2.8%, or at least about 2.9%, by weight, of the total fill weight of the composition. For example, a high dose endoxifen composition may comprise at least about 2% croscarmellose sodium, by weight, of the total fill weight of the composition.

In some embodiments, the disintegrant (e.g., croscarmellose sodium) may be included in a high dose endoxifen composition of the present disclosure at an amount of from about 0.1% to about 10%, from about 1.0% to about 10%, from about 1.5% to about 10%, from about 2.0% to about 10%, from about 2.5% to about 10%, from about 2.6% to about 10%, from about 2.8% to about 10%, from about 2.9% to about 10%, from about 0.1% to about 8.0%, from about 1.0% to about 8.0%, from about 1.5% to about 8.0%, from about 2.0% to about 8.0%, from about 2.5% to about 8.0%, from about 2.6% to about 8.0%, from about 2.8% to about 8.0%, from about 2.9% to about 8.0%, from about 0.1% to about 5.0%, from about 1.0% to about 5.0%, from about 1.5% to about 5.0%, from about 2.0% to about 5.0%, from about 2.5% to about 5.0%, from about 2.6% to about 5.0%, from about 2.8% to about 5.0%, from about 2.9% to about 5.0%, from about 0.1% to about 4.0%, from about 1.0% to about 4.0%, from about 1.5% to about 4.0%, from about 2.0% to about 4.0%, from about 2.5% to about 4.0%, from about 2.6% to about 4.0%, from about 2.8% to about 4.0%, from about 2.9% to about 4.0%, from about 0.1% to about 3.0%, from about 1.0% to about 3.0%, from about 1.5% to about 3.0%, from about 2.0% to about 3.0%, from about 2.5% to about 3.0%, from about 2.6% to about 3.0%, from about 2.8% to about 3.0%, or from about 2.9% to about 3.0%, by weight, of the total fill weight of the composition. For example, a high dose endoxifen composition may comprise from about 1% to about 4% croscarmellose sodium, by weight, of the total fill weight of the composition.

In some embodiments, a filler may be included in a high dose endoxifen composition of the present disclosure. Examples of fillers include, but are not limited to, microcrystalline cellulose, talc, calcium carbonate (e.g., granules or powder), sugars such as dextrose, sucrose, lactose, a salt such as calcium carbonate, calcium phosphate, sodium carbonate, sodium phosphate, starches, powdered cellulose, cellulosic bases such as methyl cellulose, carboxymethyl cellulose dextrates, kaolin, mannitol, silicic acid, sorbitol, starch, pregelatinized starch, and mixtures thereof.

The filler (e.g., microcrystalline cellulose) may be included in a high dose endoxifen composition of the present disclosure at an amount of from about 10% to about 99%, from about 20% to about 99%, from about 30% to about 99%, from about 40% to about 99%, from about 50% to about 99%, from about 60% to about 99%, from about 70% to about 99%, from about 75% to about 99%, from about 10% to about 97%, from about 20% to about 97%, from about 30% to about 97%, from about 40% to about 97%, from about 50% to about 97%, from about 60% to about 97%, from about 70% to about 97%, from about 75% to about 97%, from about 10% to about 95%, from about 20% to about 95%, from about 30% to about 95%, from about 40% to about 95%, from about 50% to about 95%, from about 60% to about 95%, from about 70% to about 95%, from about 75% to about 95%, from about 10% to about 90%, from about 20% to about 90%, from about 30% to about 90%, from about 40% to about 90%, from about 50% to about 90%, from about 60% to about 90%, from about 70% to about 90%, from about 75% to about 90%, from about 10% to about 80%, from about 20% to about 80%, from about 30% to about 80%, from about 40% to about 80%, from about 50% to about 80%, from about 60% to about 80%, from about 70% to about 80%, or from about 75% to about 80%, by weight, of the total fill weight of the composition. For example, a high dose endoxifen composition may comprise from about 50% to about 95% microcrystalline cellulose, by weight, of the total fill weight of the composition.

In some embodiments, a lubricant may be included in a high dose endoxifen composition of the present disclosure. Examples of lubricants include, but are not limited to, magnesium stearate, calcium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, magnesium stearate or potassium stearate, ethyl oleate, ethyl laureate, agar, and combinations thereof. Additional lubricants include, for example, a syloid silica gel (e.g., AEROSIL® 200, manufactured by W.R. GRACE® Co. of Baltimore, Md.), a coagulated aerosol of synthetic silica (e.g., marketed by DEGUSSA® Co. of Plano, Tex.), a fumed silica (e.g., CAB-O-SIL®, a pyrogenic silicon dioxide product sold by CABOT© Co. of Boston, Mass.), a silicon fluid (e.g., Q7-9120 manufactured by DOW CORNING®), and combinations thereof.

The lubricant (e.g., magnesium stearate) may be included in a high dose endoxifen composition of the present disclosure at an amount of at least about 0.10%, at least about 0.2%, at least about 0.3%, at least about 0.4%, at least about 0.5%, at least about 0.5%, at least about 0.6%, at least about 0.8%, at least about 0.8%, or at least about 1.0%, by weight, of the total fill weight of the composition. For example, a high dose endoxifen composition may comprise at least about 0.8% magnesium stearate, by weight, of the total fill weight of the composition.

In some embodiments, the lubricant (e.g., magnesium stearate) may be included in a high dose endoxifen composition of the present disclosure at an amount of from about 0.1% to about 5.0%, from about 0.2% to about 5.0%, from about 0.3% to about 5.0%, from about 0.4% to about 5.0%, from about 0.5% to about 5.0%, from about 0.6% to about 5.0%, from about 0.7% to about 5.0%, from about 0.8% to about 5.0%, from about 0.9% to about 5.0%, from about 1.0% to about 5.0%, from about 0.1% to about 4.0%, from about 0.2% to about 4.0%, from about 0.3% to about 4.0%, from about 0.4% to about 4.0%, from about 0.5% to about 4.0%, from about 0.6% to about 4.0%, from about 0.7% to about 4.0%, from about 0.8% to about 4.0%, from about 0.9% to about 4.0%, from about 1.0% to about 4.0%, from about 0.1% to about 3.0%, from about 0.2% to about 3.0%, from about 0.3% to about 3.0%, from about 0.4% to about 3.0%, from about 0.5% to about 3.0%, from about 0.6% to about 3.0%, from about 0.7% to about 3.0%, from about 0.8% to about 3.0%, from about 0.9% to about 3.0%, from about 1.0% to about 3.0%, from about 0.1% to about 2.0%, from about 0.2% to about 2.0%, from about 0.3% to about 2.0%, from about 0.4% to about 2.0%, from about 0.5% to about 2.0%, from about 0.6% to about 2.0%, from about 0.7% to about 2.0%, from about 0.8% to about 2.0%, from about 0.9% to about 2.0%, from about 1.0% to about 2.0%, from about 0.1% to about 1.5%, from about 0.2% to about 1.5%, from about 0.3% to about 1.5%, from about 0.4% to about 1.5%, from about 0.5% to about 1.5%, from about 0.6% to about 1.5%, from about 0.7% to about 1.5%, from about 0.8% to about 1.5%, from about 0.9% to about 1.5%, from about 1.0% to about 1.5%, from about 0.1% to about 1.2%, from about 0.2% to about 1.2%, from about 0.3% to about 1.2%, from about 0.4% to about 1.2%, from about 0.5% to about 1.2%, from about 0.6% to about 1.2%, from about 0.7% to about 1.2%, from about 0.8% to about 1.2%, from about 0.9% to about 1.2%, or from about 1.0% to about 1.2%, by weight, of the total fill weight of the composition. For example, a high dose endoxifen composition may comprise from about 0.5% to about 2% magnesium stearate, by weight, of the total fill weight of the composition.

Additional agents that may be included in the high dose endoxifen compositions of the present disclosure include binders, fillers, lubricants, and other pharmaceutically acceptable excipients. Binders suitable for use in the pharmaceutical compositions provided herein include, but are not limited to, sucrose, starches such as corn starch, potato starch, or starches such as starch paste, pregelatinized starch, and starch 1500, PEG 6000, methocel, WALOCEL® HM, LUVITEC®, caparolactam, AVICEL®, SMCC, UNI-PURE®, gelatin, natural and synthetic gums such as acacia, sodium alginate, alginic acid, other alginates, tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, polyvinyl pyrrolidone, hydroxypropyl methyl cellulose, (e.g., Nos. 2208, 2906, 2910), microcrystalline cellulose, and mixtures thereof. Suitable forms of microcrystalline cellulose include, but are not limited to, the materials sold as AVICEL® PH 101, AVICEL® PH 103 AVICEL® RC 581, AVICEL® PH 105 (available from FMC Corporation, American Viscose Division, Avicel Sales, Marcus Hook, Pa.), and mixtures thereof. In some embodiments, the binder is a mixture of microcrystalline cellulose and sodium carboxymethyl cellulose. Suitable anhydrous or low moisture excipients or additives include AVICEL® PH 103 and Starch 1500 LM.

An example of a high dose endoxifen composition may comprise from about 10% to about 30% (Z)-endoxifen by weight, from about 1% to about 5% croscarmellose sodium by weight, from about 60% to about 95% microcrystalline cellulose by weight, and from about 0.5% to about 3% magnesium stearate by weight, relative to total fill weight of the composition. The composition may be encapsulated in an enteric-resistant delayed release capsule, such as a capsule comprising from about 85% to about 97% hydroxypropyl methylcellulose and from about 3% to about 10% gellan gum. In some embodiments, the high dose endoxifen formulation may be formulated in a dosage form comprising about 10 mg, about 20 mg, about 40 mg, or about 80 mg of (Z)-endoxifen per dosage unit (e.g., per capsule).

A further example of a high dose endoxifen composition may comprise from about 15% to about 20% (Z)-endoxifen by weight, from about 2% to about 4% croscarmellose sodium by weight, from about 70% to about 80% microcrystalline cellulose by weight, and from about 0.5% to about 2% magnesium stearate by weight, relative to total fill weight of the composition. The composition may be encapsulated in an enteric-resistant delayed release capsule, such as a capsule comprising from about 85% to about 97% hydroxypropyl methylcellulose and from about 3% to about 7% gellan gum. In some embodiments, the high dose endoxifen formulation may be formulated in a dosage form comprising about 10 mg, about 20 mg, about 40 mg, or about 80 mg of (Z)-endoxifen per dosage unit (e.g., per capsule).

### Endoxifen Compositions

Endoxifen compositions of the present disclosure may comprise (Z)-endoxifen (e.g., Compound E1, or a pharmaceutically acceptable salt, solvate, or tautomer thereof) and other endoxifen-related compounds (e.g., Compound S1, Compound S2, Compound S3, Compound 1, Compound 2, Compound 3, Compound 4a, Compound 4b, Compound 5a, Compound 5b, Compound 6a, Compound 6b, Compound 7a, Compound 7b, Compound 8, or pharmaceutically acceptable salts, solvates, or tautomers thereof). In some embodiments, the endoxifen composition comprises Compound E1, or a pharmaceutically acceptable salt, solvate, or tautomer thereof, and at least one compound selected from: Compound 1, Compound 2, Compound 3, Compound 4a, Compound 4b, Compound 5a, Compound 5b, Compound 6a, Compound 6b, Compound 7a, Compound 7b, Compound 8, or pharmaceutically acceptable salts, solvates, or tautomers thereof. In some embodiments, the endoxifen composition comprises Compound E1, or a pharmaceutically acceptable salt, solvate, or tautomer thereof, and at least two compounds selected from: Compound 1, Compound 2, Compound 3, Compound 4a, Compound 4b, Compound 5a, Compound 5b, Compound 6a, Compound 6b, Compound 7a, Compound 7b, Compound 8, or pharmaceutically acceptable salts, solvates, or tautomers thereof. In some embodiments, the endoxifen composition comprises Compound E1, or a pharmaceutically acceptable salt, solvate, or tautomer thereof, and at least three compounds selected from: Compound 1, Compound 2, Compound 3, Compound 4a, Compound 4b, Compound 5a, Compound 5b, Compound 6a, Compound 6b, Compound 7a, Compound 7b, Compound 8, or pharmaceutically acceptable salts, solvates, or tautomers thereof. In some embodiments, the endoxifen composition comprises Compound E1, or a pharmaceutically acceptable salt, solvate, or tautomer thereof, and at least four compounds selected from: Compound 1, Compound 2, Compound 3, Compound 4a, Compound 4b, Compound 5a, Compound 5b, Compound 6a, Compound 6b, Compound 7a, Compound 7b, Compound 8, or pharmaceutically acceptable salts, solvates, or tautomers thereof. In some embodiments, the endoxifen composition comprises Compound E1, or a pharmaceutically acceptable salt, solvate, or tautomer thereof, and at least five compounds selected from: Compound 1, Compound 2, Compound 3, Compound 4a, Compound 4b, Compound 5a, Compound 5b, Compound 6a, Compound 6b, Compound 7a, Compound 7b, Compound 8, or pharmaceutically acceptable salts, solvates, or tautomers thereof. In some embodiments, the endoxifen composition comprises Compound E1, or a pharmaceutically acceptable salt, solvate, or tautomer thereof, and at least six compounds selected from: Compound 1, Compound 2, Compound 3, Compound 4a, Compound 4b, Compound 5a, Compound 5b, Compound 6a, Compound 6b, Compound 7a, Compound 7b, Compound 8, or pharmaceutically acceptable salts, solvates, or tautomers thereof. In some embodiments, the endoxifen composition comprises Compound E1, or a pharmaceutically acceptable salt, solvate, or tautomer thereof, and at least seven compounds selected from: Compound 1, Compound 2, Compound 3, Compound 4a, Compound 4b, Compound 5a, Compound 5b, Compound 6a, Compound 6b, Compound 7a, Compound 7b, Compound 8, or pharmaceutically acceptable salts, solvates, or tautomers thereof. In some embodiments, the endoxifen composition comprises Compound E1, or a pharmaceutically acceptable salt, solvate, or tautomer thereof, and at least eight compounds selected from: Compound 1, Compound 2, Compound 3, Compound 4a, Compound 4b, Compound 5a, Compound 5b, Compound 6a, Compound 6b, Compound 7a, Compound 7b, Compound 8, or pharmaceutically acceptable salts, solvates, or tautomers thereof.

Endoxifen compositions of the current disclosure may comprise at least 90% (Z)-endoxifen

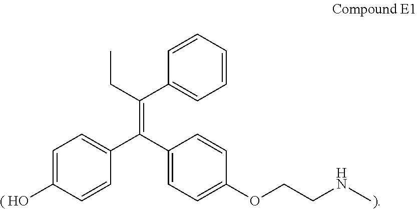

In some embodiments, an endoxifen composition of the current disclosure may comprise at least 90%, at least 90.5%, at least 91%, at least 91.5%, at least 92%, at least 92.5%, at least 93%, at least 93.5%, at least 94%, at least 94.5%, at least 95%, at least 95.25%, at least 95.5%, at least 95.75%, at least 96%, at least 96.25%, at least 96.5%, at least 96.75%, at least 97%, at least 97.25%, at least 97.5%, at least 97.75%, at least 98%, at least 98.25%, at least 98.5%, at least 98.75%, at least 99%, at least 99.1%, at least 99.2%, at least 99.3%, at least 99.4%, at least 99.5%, at least 99.6%, at least 99.7%, at least 99.75%, at least 99.8%, at least 99.85%, at least 99.9%, at least 99.91%, at least 99.92%, at least 99.93%, at least 99.94%, at least 99.95%, at least 99.96%, at least 99.97%, at least 99.98%, or at least 99.99% (Z)-endoxifen (Compound E1). In some embodiments, an endoxifen composition may comprise between 50% and 60%, between 50% and 70%, between 50% and 75%, between 50% and 80%, between 50% and 85%, between 50% and 90%, between 50% and 95%, between 50% and 97.5%, between 50% and 99%, between 50% and 99.5%, between 60% and 70%, between 60% and 75%, between 60% and 80%, between 60% and 85%, between 60% and 90%, between 60% and 95%, between 60% and 97.5%, between 60% and 99%, between 60% and 99.5%, between 70% and 75%, between 70% and 80%, between 70% and 85%, between 70% and 90%, between 70% and 95%, between 70% and 97.5%, between 70% and 99%, between 70% and 99.5%, between 75% and 80%, between 75% and 85%, between 75% and 90%, between 75% and 95%, between 75% and 97.5%, between 75% and 99%, between 75% and 99.5%, between 80% and 85%, between 80% and 90%, between 80% and 95%, between 80% and 97.5%, between 80% and 99%, between 80% and 99.5%, between 85% and 90%, between 85% and 95%, between 85% and 97.5%, between 85% and 99%, between 85% and 99.5%, between 90% and 95%, between 90% and 97.5%, between 90% and 99%, between 90% and 99.5%, between 95% and 97.5%, between 95% and 99%, between 95% and 99.5%, between 97.5% and 99.5%, not less than 95%, not less than 96%, not less than 97%, not less than 98%, not less than 99%, or not less than 99.5% (Z)-endoxifen. In some embodiments, an endoxifen composition of the current disclosure may comprise not less than 90% and not more than 92.5%, not less than 92.5% and not more than 95%, not less than 95% and not more than 96%, not less than 96% and not more than 97%, not less than 97.0% and not more than 97.5%, not less than 97.5% and not more than 98%, not less than 98.0% and not more than 98.5%, not less than 98.5% and not more than 99.0%, not less than 99.0% and not more than 99.1%, not less than 99.1% and not more than 99.2%, not less than 99.2% and not more than 99.3%, not less than 99.3% and not more than 99.4%, not less than 99.4% and not more than 99.5%, not less than 99.5% and not more than 99.6%, not less than 99.6% and not more than 99.7%, not less than 99.70% and not more than 99.75%, not less than 99.75% and not more than 99.80%, not less than 99.80% and not more than 99.85%, not less than 99.85% and not more than 99.90%, not less than 99.90% and not more than 99.91%, not less than 99.91% and not more than 99.92%, not less than 99.92% and not more than 99.93%, not less than 99.93% and not more than 99.94%, not less than 99.94% and not more than 99.95%, not less than 99.95% and not more than 99.96%, not less than 99.96% and not more than 99.97%, not less than 99.97% and not more than 99.98%, or not less than 99.98% and not more than 99.99% (Z)-endoxifen (Compound E1).

The endoxifen compositions of the present disclosure may further comprise one or more endoxifen-related compounds (e.g., any one of Compound S1, Compound S2, Compound S3, Compound 1, Compound 2, Compound 3, Compound 4a, Compound 4b, Compound 5a, Compound 5b, Compound 6a, Compound 6b, Compound 7a, Compound 7b, Compound 8, or pharmaceutically acceptable salts, solvates, or tautomers thereof). In some embodiments, an endoxifen composition of the current disclosure may comprise at most 10%, at most 9.5%, at most 9%, at most 8.5%, at most 8%, at most 7.5%, at most 7%, at most 6.5%, at most 6%, at most 5.5%, at most 5%, at most 4.75%, at most 4.5%, at most 4.25%, at most 4%, at most 3.75%, at most 3.5%, at most 3.25%, at most 3%, at most 2.75%, at most 2.5%, at most 2.25%, at most 2%, at most 1.75%, at most 1.5%, at most 1.25%, at most 1%, at most 0.9%, at most 0.8%, at most 0.7%, at most 0.6%, at most 0.5%, at most 0.4%, at most 0.3%, at most 0.25%, at most 0.2%, at most 0.15%, at most 0.1%, at most 0.09%, at most 0.08%, at most 0.07%, at most 0.06%, at most 0.05%, at most 0.04%, at most 0.03%, at most 0.02%, or at most 0.01% of one or endoxifen-related compounds (e.g., any one of Compound S1, Compound S2, Compound S3, Compound 1, Compound 2, Compound 3, Compound 4a, Compound 4b, Compound 5a, Compound 5b, Compound 6a, Compound 6b, Compound 7a, Compound 7b, Compound 8, or pharmaceutically acceptable salts, solvates, or tautomers thereof). In some embodiments, an endoxifen composition of the current disclosure may comprise not less than 0.01% and not more than 0.1%, not less than 0.01% and not more than 0.15%, not less than 0.01% and not more than 0.2%, not less than 0.01% and not more than 0.25%, not less than 0.01% and not more than 0.3%, not less than 0.01% and not more than 0.4%, not less than 0.01% and not more than 0.5%, not less than 0.01% and not more than 0.6%, not less than 0.01% and not more than 0.7%, not less than 0.01% and not more than 0.8%, not less than 0.01% and not more than 0.9%, not less than 0.01% and not more than 1%, not less than 0.01% and not more than 5%, not less than 0.01% and not more than 10%, not less than 0.1% and not more than 0.15%, not less than 0.1% and not more than 0.2%, not less than 0.10% and not more than 0.25%, not less than 0.1% and not more than 0.3%, not less than 0.1% and not more than 0.4%, not less than 0.1% and not more than 0.5%, not less than 0.10% and not more than 0.6%, not less than 0.10% and not more than 0.7%, not less than 0.10% and not more than 0.8%, not less than 0.10% and not more than 0.9%, not less than 0.1% and not more than 1%, not less than 0.1% and not more than 1.25%, not less than 0.1% and not more than 1.5%, not less than 0.1% and not more than 1.75%, not less than 0.1% and not more than 2%, not less than 0.1% and not more than 5%, not less than 0.1% and not more than 10%, not less than 0.25% and not more than 0.5%, not less than 0.25% and not more than 0.6%, not less than 0.25% and not more than 0.7%, not less than 0.25% and not more than 0.8%, not less than 0.25% and not more than 0.9%, not less than 0.25% and not more than 1%, not less than 0.25% and not more than 1.25%, not less than 0.25% and not more than 1.5%, not less than 0.25% and not more than 1.75%, not less than 0.25% and not more than 2%, not less than 0.25% and not more than 2.25%, not less than 0.25% and not more than 2.5%, not less than 0.25% and not more than 5%, not less than 0.25% and not more than 10%, not less than 0.5% and not more than 0.6%, not less than 0.5% and not more than 0.7%, not less than 0.5% and not more than 0.8%, not less than 0.5% and not more than 0.9%, not less than 0.5% and not more than 1%, not less than 0.5% and not more than 1.25%, not less than 0.5% and not more than 1.5%, not less than 0.5% and not more than 1.75%, not less than 0.5% and not more than 2%, not less than 0.5% and not more than 2.25%, not less than 0.5% and not more than 2.5%, not less than 0.5% and not more than 2.75%, not less than 0.5% and not more than 3%, not less than 0.5% and not more than 3.25%, not less than 0.5% and not more than 3.5%, not less than 0.5% and not more than 3.75%, not less than 0.5% and not more than 4%, not less than 0.5% and not more than 4.25%, not less than 0.5% and not more than 4.5%, not less than 0.5% and not more than 4.75%, not less than 0.5% and not more than 5%, not less than 0.5% and not more than 10%, not less than 0.75% and not more than 1%, not less than 0.75% and not more than 1.25%, not less than 0.75% and not more than 1.5%, not less than 0.75% and not more than 1.75%, not less than 0.75% and not more than 2%, not less than 0.75% and not more than 2.25%, not less than 0.75% and not more than 2.5%, not less than 0.75% and not more than 2.75%, not less than 0.75% and not more than 3%, not less than 0.75% and not more than 3.25%, not less than 0.75% and not more than 3.5%, not less than 0.75% and not more than 3.75%, not less than 0.75% and not more than 4%, not less than 0.75% and not more than 4.25%, not less than 0.75% and not more than 4.5%, not less than 0.75% and not more than 4.75%, not less than 0.75% and not more than 5%, not less than 0.75% and not more than 5.5%, not less than 0.75% and not more than 6%, not less than 0.75% and not more than 6.5%, not less than 0.75% and not more than 7%, not less than 0.75% and not more than 7.5%, not less than 0.75% and not more than 10%, not less than 1% and not more than 1.25%, not less than 1% and not more than 1.5%, not less than 1% and not more than 1.75%, not less than 1% and not more than 2%, not less than 1% and not more than 2.25%, not less than 1% and not more than 2.5%, not less than 1% and not more than 2.75%, not less than 1% and not more than 3%, not less than 1% and not more than 3.25%, not less than 1% and not more than 3.5%, not less than 1% and not more than 3.75%, not less than 1% and not more than 4%, not less than 1% and not more than 4.25%, not less than 1% and not more than 4.5%, not less than 1% and not more than 4.75%, not less than 1% and not more than 5%, not less than 1% and not more than 5.5%, not less than 1% and not more than 6%, not less than 1% and not more than 6.5%, not less than 1% and not more than 7%, not less than 1% and not more than 7.5%, not less than 1% and not more than 8%, not less than 1% and not more than 8.5%, not less than 1% and not more than 9%, not less than 1% and not more than 9.5%, not less than 1% and not more than 10%, not less than 1.5% and not more than 1.75%, not less than 1.5% and not more than 2%, not less than 1.5% and not more than 2.25%, not less than 1.5% and not more than 2.5%, not less than 1.5% and not more than 2.75%, not less than 1.5% and not more than 3%, not less than 1.5% and not more than 3.25%, not less than 1.5% and not more than 3.5%, not less than 1.5% and not more than 3.75%, not less than 1.5% and not more than 4%, not less than 1.5% and not more than 4.25%, not less than 1.5% and not more than 4.5%, not less than 1.5% and not more than 4.75%, not less than 1.5% and not more than 5%, not less than 1.5% and not more than 5.5%, not less than 1.5% and not more than 6%, not less than 1.5% and not more than 6.5%, not less than 1.5% and not more than 7%, not less than 1.5% and not more than 7.5%, not less than 1.5% and not more than 8%, not less than 1.5% and not more than 8.5%, not less than 1.5% and not more than 9%, not less than 1.5% and not more than 9.5%, not less than 1.5% and not more than 10%, not less than 2% and not more than 2.25%, not less than 2% and not more than 2.5%, not less than 2% and not more than 2.75%, not less than 2% and not more than 3%, not less than 2% and not more than 3.25%, not less than 2% and not more than 3.5%, not less than 2% and not more than 3.75%, not less than 2% and not more than 4%, not less than 2% and not more than 4.25%, not less than 2% and not more than 4.5%, not less than 2% and not more than 4.75%, not less than 2% and not more than 5%, not less than 2% and not more than 5.5%, not less than 2% and not more than 6%, not less than 2% and not more than 6.5%, not less than 2% and not more than 7%, not less than 2% and not more than 7.5%, not less than 2% and not more than 8%, not less than 2% and not more than 8.5%, not less than 2% and not more than 9%, not less than 2% and not more than 9.5%, not less than 2% and not more than 10%, not less than 2.5% and not more than 3%, not less than 2.5% and not more than 3.25%, not less than 2.5% and not more than 3.5%, not less than 2.5% and not more than 3.75%, not less than 2.5% and not more than 4%, not less than 2.5% and not more than 4.25%, not less than 2.5% and not more than 4.5%, not less than 2.5% and not more than 4.75%, not less than 2.5% and not more than 5%, not less than 2.5% and not more than 5.5%, not less than 2.5% and not more than 6%, not less than 2.5% and not more than 6.5%, not less than 2.5% and not more than 7%, not less than 2.5% and not more than 7.5%, not less than 2.5% and not more than 8%, not less than 2.5% and not more than 8.5%, not less than 2.5% and not more than 9%, not less than 2.5% and not more than 9.5%, not less than 2.5% and not more than 10%, not less than 3% and not more than 3.5%, not less than 3% and not more than 3.75%, not less than 3% and not more than 4%, not less than 3% and not more than 4.25%, not less than 3% and not more than 4.5%, not less than 3% and not more than 4.75%, not less than 3% and not more than 5%, not less than 3% and not more than 5.5%, not less than 3% and not more than 6%, not less than 3% and not more than 6.5%, not less than 3% and not more than 7%, not less than 3% and not more than 7.5%, not less than 3% and not more than 8%, not less than 3% and not more than 8.5%, not less than 3% and not more than 9%, not less than 3% and not more than 9.5%, not less than 3% and not more than 10%, not less than 4% and not more than 5%, not less than 4% and not more than 5.5%, not less than 4% and not more than 6%, not less than 4% and not more than 6.5%, not less than 4% and not more than 7%, not less than 4% and not more than 7.5%, not less than 4% and not more than 8%, not less than 4% and not more than 8.5%, not less than 4% and not more than 9%, not less than 4% and not more than 9.5%, not less than 4% and not more than 10%, not less than 5% and not more than 5.5%, not less than 5% and not more than 6%, not less than 5% and not more than 6.5%, not less than 5% and not more than 7%, not less than 5% and not more than 7.5%, not less than 5% and not more than 8%, not less than 5% and not more than 8.5%, not less than 5% and not more than 9%, not less than 5% and not more than 9.5%, or not less than 5% and not more than 10% w/w of one or endoxifen-related compounds (e.g., any one of Compound S1, Compound S2, Compound S3, Compound 1, Compound 2, Compound 3, Compound 4a, Compound 4b, Compound 5a, Compound 5b, Compound 6a, Compound 6b, Compound 7a, Compound 7b, Compound 8, or pharmaceutically acceptable salts, solvates, or tautomers thereof). In some embodiments, the endoxifen composition comprises not less than 0.01% and not more than 0.1%, not less than 0.1% and not more than 1%, not less than 0.25% and not more than 1%, not less than 0.5% and not more than 1.5%, not less than 0.5% and not more than 2.5%, not less than 0.5% and not more than 5%, not less than 1% and not more than 2%, not less than 1% and not more than 2.25%, not less than 1% and not more than 2.5%, not less than 1% and not more than 3.75%, not less than 1% and not more than 4%, not less than 1% and not more than 4.25%, not less than 1% and not more than 4.5%, not less than 1% and not more than 4.75%, not less than 1% and not more than 5%, not less than 1% and not more than 5.5%, not less than 1% and not more than 6%, not less than 1% and not more than 6.5%, not less than 1% and not more than 7%, or not less than 1% and not more than 7.5% w/w of one or more compounds selected from the group consisting of. Compound 1, Compound 2, Compound 3, Compound 4a, Compound 4b, Compound 5a, Compound 5b, Compound 6a, Compound 6b, Compound 7a, and Compound 7b, or pharmaceutically acceptable salts, solvates, or tautomers thereof.

In some embodiments, an endoxifen composition of the current disclosure may comprise at least 90% (Z)-endoxifen (Compound E1) and not less than 0.1% and not more than 10%, not less than 0.1% and not more than 9.5%, not less than 0.1% and not more than 9%, not less than 0.1% and not more than 8.5%, not less than 0.1% and not more than 8%, not less than 0.1% and not more than 7.5%, not less than 0.1% and not more than 7%, not less than 0.1% and not more than 6.5%, not less than 0.1% and not more than 6%, not less than 0.1% and not more than 5.5%, not less than 0.1% and not more than 5%, not less than 0.1% and not more than 4.75%, not less than 0.1% and not more than 4.5%, not less than 0.1% and not more than 4.25%, not less than 0.1% and not more than 4%, not less than 0.1% and not more than 3.75%, not less than 0.1% and not more than 3.5%, not less than 0.1% and not more than 3.25%, not less than 0.1% and not more than 3%, not less than 0.1% and not more than 2.75%, not less than 0.1% and not more than 2.5%, not less than 0.1% and not more than 2.25%, not less than 0.1% and not more than 2%, not less than 0.1% and not more than 1.75%, not less than 0.1% and not more than 1.5%, not less than 0.1% and not more than 1.25%, not less than 0.01% and not more than 1%, not less than 0.01% and not more than 0.9%, not less than 0.01% and not more than 0.8%, not less than 0.01% and not more than 0.7%, not less than 0.01% and not more than 0.6%, not less than 0.01% and not more than 0.5%, not less than 0.01% and not more than 0.4%, not less than 0.01% and not more than 0.3%, not less than 0.01% and not more than 0.25%, not less than 0.01% and not more than 0.2%, not less than 0.001% and not more than 0.15%, not less than 0.001% and not more than 0.1%, not less than 0.001% and not more than 0.09%, not less than 0.001% and not more than 0.08%, not less than 0.001% and not more than 0.07%, not less than 0.001% and not more than 0.06%, not less than 0.001% and not more than 0.05%, not less than 0.001% and not more than 0.04%, not less than 0.001% and not more than 0.03%, not less than 0.001% and not more than 0.02%, or not less than 0.001% and not more than 0.01% of Compound 1, or a pharmaceutically acceptable salt, solvate, or tautomer thereof.

In some embodiments, an endoxifen composition of the current disclosure may comprise at least 90% (Z)-endoxifen (Compound E1) and not less than 0.1% and not more than 10%, not less than 0.1% and not more than 9.5%, not less than 0.1% and not more than 9%, not less than 0.1% and not more than 8.5%, not less than 0.1% and not more than 8%, not less than 0.1% and not more than 7.5%, not less than 0.1% and not more than 7%, not less than 0.1% and not more than 6.5%, not less than 0.1% and not more than 6%, not less than 0.1% and not more than 5.5%, not less than 0.1% and not more than 5%, not less than 0.1% and not more than 4.75%, not less than 0.1% and not more than 4.5%, not less than 0.1% and not more than 4.25%, not less than 0.1% and not more than 4%, not less than 0.1% and not more than 3.75%, not less than 0.1% and not more than 3.5%, not less than 0.1% and not more than 3.25%, not less than 0.1% and not more than 3%, not less than 0.1% and not more than 2.75%, not less than 0.1% and not more than 2.5%, not less than 0.1% and not more than 2.25%, not less than 0.1% and not more than 2%, not less than 0.1% and not more than 1.75%, not less than 0.1% and not more than 1.5%, not less than 0.1% and not more than 1.25%, not less than 0.01% and not more than 1%, not less than 0.01% and not more than 0.9%, not less than 0.01% and not more than 0.8%, not less than 0.01% and not more than 0.7%, not less than 0.01% and not more than 0.6%, not less than 0.01% and not more than 0.5%, not less than 0.01% and not more than 0.4%, not less than 0.01% and not more than 0.3%, not less than 0.01% and not more than 0.25%, not less than 0.01% and not more than 0.2%, not less than 0.001% and not more than 0.15%, not less than 0.001% and not more than 0.1%, not less than 0.001% and not more than 0.09%, not less than 0.001% and not more than 0.08%, not less than 0.001% and not more than 0.07%, not less than 0.001% and not more than 0.06%, not less than 0.001% and not more than 0.05%, not less than 0.001% and not more than 0.04%, not less than 0.001% and not more than 0.03%, not less than 0.001% and not more than 0.02%, or not less than 0.001% and not more than 0.01% of Compound 2, or a pharmaceutically acceptable salt, solvate, or tautomer thereof.

In some embodiments, an endoxifen composition of the current disclosure may comprise at least 90% (Z)-endoxifen (Compound E1) and not less than 0.1% and not more than 10%, not less than 0.1% and not more than 9.5%, not less than 0.1% and not more than 9%, not less than 0.1% and not more than 8.5%, not less than 0.1% and not more than 8%, not less than 0.1% and not more than 7.5%, not less than 0.1% and not more than 7%, not less than 0.1% and not more than 6.5%, not less than 0.1% and not more than 6%, not less than 0.1% and not more than 5.5%, not less than 0.1% and not more than 5%, not less than 0.1% and not more than 4.75%, not less than 0.1% and not more than 4.5%, not less than 0.1% and not more than 4.25%, not less than 0.1% and not more than 4%, not less than 0.1% and not more than 3.75%, not less than 0.1% and not more than 3.5%, not less than 0.1% and not more than 3.25%, not less than 0.1% and not more than 3%, not less than 0.1% and not more than 2.75%, not less than 0.1% and not more than 2.5%, not less than 0.1% and not more than 2.25%, not less than 0.1% and not more than 2%, not less than 0.1% and not more than 1.75%, not less than 0.1% and not more than 1.5%, not less than 0.1% and not more than 1.25%, not less than 0.01% and not more than 1%, not less than 0.01% and not more than 0.9%, not less than 0.01% and not more than 0.8%, not less than 0.01% and not more than 0.7%, not less than 0.01% and not more than 0.6%, not less than 0.01% and not more than 0.5%, not less than 0.01% and not more than 0.4%, not less than 0.01% and not more than 0.3%, not less than 0.01% and not more than 0.25%, not less than 0.01% and not more than 0.2%, not less than 0.001% and not more than 0.15%, not less than 0.001% and not more than 0.1%, not less than 0.001% and not more than 0.09%, not less than 0.001% and not more than 0.08%, not less than 0.001% and not more than 0.07%, not less than 0.001% and not more than 0.06%, not less than 0.001% and not more than 0.05%, not less than 0.001% and not more than 0.04%, not less than 0.001% and not more than 0.03%, not less than 0.0010% and not more than 0.02%, or not less than 0.001% and not more than 0.01% of Compound 3, or a pharmaceutically acceptable salt, solvate, or tautomer thereof.

In some embodiments, an endoxifen composition of the current disclosure may comprise at least 90% (Z)-endoxifen (Compound E1) and not less than 0.1% and not more than 10%, not less than 0.1% and not more than 9.5%, not less than 0.1% and not more than 9%, not less than 0.1% and not more than 8.5%, not less than 0.1% and not more than 8%, not less than 0.1% and not more than 7.5%, not less than 0.1% and not more than 7%, not less than 0.1% and not more than 6.5%, not less than 0.1% and not more than 6%, not less than 0.1% and not more than 5.5%, not less than 0.1% and not more than 5%, not less than 0.1% and not more than 4.75%, not less than 0.1% and not more than 4.5%, not less than 0.1% and not more than 4.25%, not less than 0.1% and not more than 4%, not less than 0.1% and not more than 3.75%, not less than 0.1% and not more than 3.5%, not less than 0.1% and not more than 3.25%, not less than 0.1% and not more than 3%, not less than 0.1% and not more than 2.75%, not less than 0.1% and not more than 2.5%, not less than 0.1% and not more than 2.25%, not less than 0.1% and not more than 2%, not less than 0.1% and not more than 1.75%, not less than 0.1% and not more than 1.5%, not less than 0.1% and not more than 1.25%, not less than 0.01% and not more than 1%, not less than 0.01% and not more than 0.9%, not less than 0.01% and not more than 0.8%, not less than 0.01% and not more than 0.7%, not less than 0.01% and not more than 0.6%, not less than 0.01% and not more than 0.5%, not less than 0.01% and not more than 0.4%, not less than 0.01% and not more than 0.3%, not less than 0.01% and not more than 0.25%, not less than 0.01% and not more than 0.2%, not less than 0.001% and not more than 0.15%, not less than 0.001% and not more than 0.1%, not less than 0.001% and not more than 0.09%, not less than 0.001% and not more than 0.08%, not less than 0.001% and not more than 0.07%, not less than 0.001% and not more than 0.06%, not less than 0.001% and not more than 0.05%, not less than 0.001% and not more than 0.04%, not less than 0.001% and not more than 0.03%, not less than 0.0010% and not more than 0.02%, or not less than 0.0010% and not more than 0.01% of Compounds 4a and 4b, or a pharmaceutically acceptable salt, solvate, or tautomer thereof.

In some embodiments, an endoxifen composition of the current disclosure may comprise at least 90% (Z)-endoxifen (Compound E1) and not less than 0.1% and not more than 10%, not less than 0.1% and not more than 9.5%, not less than 0.1% and not more than 9%, not less than 0.1% and not more than 8.5%, not less than 0.1% and not more than 8%, not less than 0.1% and not more than 7.5%, not less than 0.1% and not more than 7%, not less than 0.1% and not more than 6.5%, not less than 0.1% and not more than 6%, not less than 0.1% and not more than 5.5%, not less than 0.1% and not more than 5%, not less than 0.1% and not more than 4.75%, not less than 0.1% and not more than 4.5%, not less than 0.1% and not more than 4.25%, not less than 0.1% and not more than 4%, not less than 0.1% and not more than 3.75%, not less than 0.1% and not more than 3.5%, not less than 0.1% and not more than 3.25%, not less than 0.1% and not more than 3%, not less than 0.1% and not more than 2.75%, not less than 0.1% and not more than 2.5%, not less than 0.1% and not more than 2.25%, not less than 0.1% and not more than 2%, not less than 0.1% and not more than 1.75%, not less than 0.1% and not more than 1.5%, not less than 0.1% and not more than 1.25%, not less than 0.01% and not more than 1%, not less than 0.01% and not more than 0.9%, not less than 0.01% and not more than 0.8%, not less than 0.01% and not more than 0.7%, not less than 0.01% and not more than 0.6%, not less than 0.01% and not more than 0.5%, not less than 0.01% and not more than 0.4%, not less than 0.01% and not more than 0.3%, not less than 0.01% and not more than 0.25%, not less than 0.01% and not more than 0.2%, not less than 0.001% and not more than 0.15%, not less than 0.001% and not more than 0.1%, not less than 0.001% and not more than 0.09%, not less than 0.001% and not more than 0.08%, not less than 0.001% and not more than 0.07%, not less than 0.001% and not more than 0.06%, not less than 0.001% and not more than 0.05%, not less than 0.001% and not more than 0.04%, not less than 0.001% and not more than 0.03%, not less than 0.001% and not more than 0.02%, or not less than 0.001% and not more than 0.01% of Compounds 5a and 5b, or a pharmaceutically acceptable salt, solvate, or tautomer thereof.

In some embodiments, an endoxifen composition of the current disclosure may comprise at least 90% (Z)-endoxifen (Compound E1) and not less than 0.1% and not more than 10%, not less than 0.1% and not more than 9.5%, not less than 0.1% and not more than 9%, not less than 0.1% and not more than 8.5%, not less than 0.1% and not more than 8%, not less than 0.1% and not more than 7.5%, not less than 0.1% and not more than 7%, not less than 0.1% and not more than 6.5%, not less than 0.1% and not more than 6%, not less than 0.1% and not more than 5.5%, not less than 0.1% and not more than 5%, not less than 0.1% and not more than 4.75%, not less than 0.1% and not more than 4.5%, not less than 0.1% and not more than 4.25%, not less than 0.1% and not more than 4%, not less than 0.1% and not more than 3.75%, not less than 0.1% and not more than 3.5%, not less than 0.1% and not more than 3.25%, not less than 0.1% and not more than 3%, not less than 0.1% and not more than 2.75%, not less than 0.1% and not more than 2.5%, not less than 0.1% and not more than 2.25%, not less than 0.1% and not more than 2%, not less than 0.1% and not more than 1.75%, not less than 0.1% and not more than 1.5%, not less than 0.1% and not more than 1.25%, not less than 0.01% and not more than 1%, not less than 0.01% and not more than 0.9%, not less than 0.01% and not more than 0.8%, not less than 0.01% and not more than 0.7%, not less than 0.01% and not more than 0.6%, not less than 0.01% and not more than 0.5%, not less than 0.01% and not more than 0.4%, not less than 0.01% and not more than 0.3%, not less than 0.01% and not more than 0.25%, not less than 0.01% and not more than 0.2%, not less than 0.001% and not more than 0.15%, not less than 0.001% and not more than 0.1%, not less than 0.001% and not more than 0.09%, not less than 0.001% and not more than 0.08%, not less than 0.001% and not more than 0.07%, not less than 0.001% and not more than 0.06%, not less than 0.001% and not more than 0.05%, not less than 0.001% and not more than 0.04%, not less than 0.001% and not more than 0.03%, not less than 0.001% and not more than 0.02%, or not less than 0.001% and not more than 0.01% of Compounds 6a and 6b, or a pharmaceutically acceptable salt, solvate, or tautomer thereof.

In some embodiments, an endoxifen composition of the current disclosure may comprise at least 90% (Z)-endoxifen (Compound E1) and not less than 0.1% and not more than 10%, not less than 0.1% and not more than 9.5%, not less than 0.1% and not more than 9%, not less than 0.1% and not more than 8.5%, not less than 0.1% and not more than 8%, not less than 0.1% and not more than 7.5%, not less than 0.1% and not more than 7%, not less than 0.1% and not more than 6.5%, not less than 0.1% and not more than 6%, not less than 0.1% and not more than 5.5%, not less than 0.1% and not more than 5%, not less than 0.1% and not more than 4.75%, not less than 0.1% and not more than 4.5%, not less than 0.1% and not more than 4.25%, not less than 0.1% and not more than 4%, not less than 0.1% and not more than 3.75%, not less than 0.1% and not more than 3.5%, not less than 0.1% and not more than 3.25%, not less than 0.1% and not more than 3%, not less than 0.1% and not more than 2.75%, not less than 0.1% and not more than 2.5%, not less than 0.1% and not more than 2.25%, not less than 0.1% and not more than 2%, not less than 0.1% and not more than 1.75%, not less than 0.1% and not more than 1.5%, not less than 0.1% and not more than 1.25%, not less than 0.01% and not more than 1%, not less than 0.01% and not more than 0.9%, not less than 0.01% and not more than 0.8%, not less than 0.01% and not more than 0.7%, not less than 0.01% and not more than 0.6%, not less than 0.01% and not more than 0.5%, not less than 0.01% and not more than 0.4%, not less than 0.01% and not more than 0.3%, not less than 0.01% and not more than 0.25%, not less than 0.01% and not more than 0.2%, not less than 0.001% and not more than 0.15%, not less than 0.001% and not more than 0.1%, not less than 0.001% and not more than 0.09%, not less than 0.001% and not more than 0.08%, not less than 0.001% and not more than 0.07%, not less than 0.001% and not more than 0.06%, not less than 0.001% and not more than 0.05%, not less than 0.001% and not more than 0.04%, not less than 0.001% and not more than 0.03%, not less than 0.001% and not more than 0.02%, or not less than 0.001% and not more than 0.01% of Compounds 7a and 7b, or a pharmaceutically acceptable salt, solvate, or tautomer thereof.

In some embodiments, an endoxifen composition of the current disclosure may comprise at least 90% (Z)-endoxifen (Compound E1) and not less than 0.1% and not more than 10%, not less than 0.1% and not more than 9.5%, not less than 0.1% and not more than 9%, not less than 0.1% and not more than 8.5%, not less than 0.1% and not more than 8%, not less than 0.1% and not more than 7.5%, not less than 0.1% and not more than 7%, not less than 0.1% and not more than 6.5%, not less than 0.1% and not more than 6%, not less than 0.1% and not more than 5.5%, not less than 0.1% and not more than 5%, not less than 0.1% and not more than 4.75%, not less than 0.1% and not more than 4.5%, not less than 0.1% and not more than 4.25%, not less than 0.1% and not more than 4%, not less than 0.1% and not more than 3.75%, not less than 0.1% and not more than 3.5%, not less than 0.1% and not more than 3.25%, not less than 0.1% and not more than 3%, not less than 0.1% and not more than 2.75%, not less than 0.1% and not more than 2.5%, not less than 0.1% and not more than 2.25%, not less than 0.1% and not more than 2%, not less than 0.1% and not more than 1.75%, not less than 0.1% and not more than 1.5%, not less than 0.1% and not more than 1.25%, not less than 0.01% and not more than 1%, not less than 0.01% and not more than 0.9%, not less than 0.01% and not more than 0.8%, not less than 0.01% and not more than 0.7%, not less than 0.01% and not more than 0.6%, not less than 0.01% and not more than 0.5%, not less than 0.01% and not more than 0.4%, not less than 0.01% and not more than 0.3%, not less than 0.01% and not more than 0.25%, not less than 0.01% and not more than 0.2%, not less than 0.001% and not more than 0.15%, not less than 0.001% and not more than 0.1%, not less than 0.001% and not more than 0.09%, not less than 0.001% and not more than 0.08%, not less than 0.001% and not more than 0.07%, not less than 0.001% and not more than 0.06%, not less than 0.001% and not more than 0.05%, not less than 0.001% and not more than 0.04%, not less than 0.001% and not more than 0.03%, not less than 0.0010% and not more than 0.02%, or not less than 0.001% and not more than 0.010% of Compound 8, or a pharmaceutically acceptable salt, solvate, or tautomer thereof.

In some embodiments, an endoxifen composition of the current disclosure may comprise at least 90%, at least 90.5%, at least 91%, at least 91.5%, at least 92%, at least 92.5%, at least 93%, at least 93.5%, at least 94%, at least 94.5%, at least 95%, at least 95.25%, at least 95.5%, at least 95.75%, at least 96%, at least 96.25%, at least 96.5%, at least 96.75%, at least 97%, at least 97.25%, at least 97.5%, at least 97.75%, at least 98%, at least 98.25%, at least 98.5%, at least 98.75%, or at least 99% (Z)-endoxifen (Compound E1) and not less than 0.001% and not more than 1% of Compound 1.

In some embodiments, an endoxifen composition of the current disclosure may comprise at least 90%, at least 9.5%, at least 91%, at least 91.5%, at least 92%, at least 92.5%, at least 93%, at least 93.5%, at least 94%, at least 94.5%, at least 95%, at least 95.25%, at least 95.5%, at least 95.75%, at least 96%, at least 96.25%, at least 96.5%, at least 96.75%, at least 97%, at least 97.25%, at least 97.5%, at least 97.75%, at least 98%, at least 98.25%, at least 98.5%, at least 98.75%, or at least 99% (Z)-endoxifen (Compound E1) and not less than 0.001% and not more than 1% of Compound 2.

In some embodiments, an endoxifen composition of the current disclosure may comprise at least 90%, at least 9.5%, at least 91%, at least 91.5%, at least 92%, at least 92.5%, at least 93%, at least 93.5%, at least 94%, at least 94.5%, at least 95%, at least 95.25%, at least 95.5%, at least 95.75%, at least 96%, at least 96.25%, at least 96.5%, at least 96.75%, at least 97%, at least 97.25%, at least 97.5%, at least 97.75%, at least 98%, at least 98.25%, at least 98.5%, at least 98.75%, or at least 99% (Z)-endoxifen (Compound E1) and not less than 0.001% and not more than 1% of Compound 3.

In some embodiments, an endoxifen composition of the current disclosure may comprise at least 90%, at least 9.5%, at least 91%, at least 91.5%, at least 92%, at least 92.5%, at least 93%, at least 93.5%, at least 94%, at least 94.5%, at least 95%, at least 95.25%, at least 95.5%, at least 95.75%, at least 96%, at least 96.25%, at least 96.5%, at least 96.75%, at least 97%, at least 97.25%, at least 97.5%, at least 97.75%, at least 98%, at least 98.25%, at least 98.5%, at least 98.75%, or at least 99% (Z)-endoxifen (Compound E1) and not less than 0.001% and not more than 1% of Compounds 4a and 4b.

In some embodiments, an endoxifen composition of the current disclosure may comprise at least 90%, at least 9.5%, at least 91%, at least 91.5%, at least 92%, at least 92.5%, at least 93%, at least 93.5%, at least 94%, at least 94.5%, at least 95%, at least 95.25%, at least 95.5%, at least 95.75%, at least 96%, at least 96.25%, at least 96.5%, at least 96.75%, at least 97%, at least 97.25%, at least 97.5%, at least 97.75%, at least 98%, at least 98.25%, at least 98.5%, at least 98.75%, or at least 99% (Z)-endoxifen (Compound E1) and not less than 0.001% and not more than 1% of Compounds 5a and 5b.

In some embodiments, an endoxifen composition of the current disclosure may comprise at least 90%, at least 90.5%, at least 91%, at least 91.5%, at least 92%, at least 92.5%, at least 93%, at least 93.5%, at least 94%, at least 94.5%, at least 95%, at least 95.25%, at least 95.5%, at least 95.75%, at least 96%, at least 96.25%, at least 96.5%, at least 96.75%, at least 97%, at least 97.25%, at least 97.5%, at least 97.75%, at least 98%, at least 98.25%, at least 98.5%, at least 98.75%, or at least 99% (Z)-endoxifen (Compound E1) and not less than 0.001% and not more than 1% of Compounds 6a and 6b.

In some embodiments, an endoxifen composition of the current disclosure may comprise at least 90%, at least 90.5%, at least 91%, at least 91.5%, at least 92%, at least 92.5%, at least 93%, at least 93.5%, at least 94%, at least 94.5%, at least 95%, at least 95.25%, at least 95.5%, at least 95.75%, at least 96%, at least 96.25%, at least 96.5%, at least 96.75%, at least 97%, at least 97.25%, at least 97.5%, at least 97.75%, at least 98%, at least 98.25%, at least 98.5%, at least 98.75%, or at least 99% (Z)-endoxifen (Compound E1) and not less than 0.001% and not more than 1% of Compounds 7a and 7b.

In some embodiments, an endoxifen composition of the current disclosure may comprise at least 90%, at least 9.5%, at least 91%, at least 91.5%, at least 92%, at least 92.5%, at least 93%, at least 93.5%, at least 94%, at least 94.5%, at least 95%, at least 95.25%, at least 95.5%, at least 95.75%, at least 96%, at least 96.25%, at least 96.5%, at least 96.75%, at least 97%, at least 97.25%, at least 97.5%, at least 97.75%, at least 98%, at least 98.25%, at least 98.5%, at least 98.75%, or at least 99% (Z)-endoxifen (Compound E1) and not less than 0.001% and not more than 1% of Compound 8.

### Pharmaceutical Compositions

Compositions of the present disclosure may comprise an endoxifen composition comprising endoxifen and one or more compounds as described herein and a pharmaceutically acceptable carrier or diluent. In some embodiments, a pharmaceutical composition may comprise an endoxifen (Compound S3) and one or more endoxifen-related compounds (e.g., any one of Compound 1-Compound 8) and a pharmaceutically acceptable carrier or diluent. In some embodiments, a pharmaceutical composition may comprise a (Z)-endoxifen (Compound E1) and one or more endoxifen-related compounds (e.g., any one of Compound 1, Compound 2, Compound 3, Compound 4a, Compound 4b, Compound 5a, Compound 5b, Compound 6a, Compound 6b, Compound 7a, Compound 7b, Compound 8, or a pharmaceutically acceptable salt, solvate, or tautomer thereof) and a pharmaceutically acceptable carrier or diluent. In some embodiments, the endoxifen composition may comprise one or more compounds selected from Compound S3, Compound E1, Compound 1, Compound 2, Compound 3, Compound 4a, Compound 4b, Compound 5a, Compound 5b, Compound 6a, Compound 6b, Compound 7a, Compound 7b, Compound 8, or a pharmaceutically acceptable salt, solvate, or tautomer thereof. In some embodiments, the endoxifen composition may comprise two or more compounds selected from Compound S3, Compound E1, Compound 1, Compound 2, Compound 3, Compound 4a, Compound 4b, Compound 5a, Compound 5b, Compound 6a, Compound 6b, Compound 7a, Compound 7b, Compound 8, or a pharmaceutically acceptable salt, solvate, or tautomer thereof.

In some embodiments, the endoxifen (e.g., Compound S3 or Compound E1), or a pharmaceutically acceptable salt, solvate, or tautomer thereof, is present in between 40-60%, 50-70%, 60-80%, 70-90%, 80-90%, 85-95%, 90-95%, or 95-99% (w/w). In some embodiments, a composition comprising one or more of the following compounds: Compound 1, Compound 2, Compound 3, Compound 4a, Compound 4b, Compound 5a, Compound 5b, Compound 6a, Compound 6b, Compound 7a, Compound 7b, Compound 8, or a pharmaceutically acceptable salt, solvate, or tautomer thereof, is present in an amount not less than 0.1% and not more than 10%, not less than 0.10% and not more than 9.5%, not less than 0.10% and not more than 9%, not less than 0.1% and not more than 8.5%, not less than 0.1% and not more than 8%, not less than 0.1% and not more than 7.5%, not less than 0.1% and not more than 7%, not less than 0.1% and not more than 6.5%, not less than 0.1% and not more than 6%, not less than 0.1% and not more than 5.5%, not less than 0.1% and not more than 5%, not less than 0.1% and not more than 4.75%, not less than 0.1% and not more than 4.5%, not less than 0.1% and not more than 4.25%, not less than 0.1% and not more than 4%, not less than 0.1% and not more than 3.75%, not less than 0.1% and not more than 3.5%, not less than 0.1% and not more than 3.25%, not less than 0.1% and not more than 3%, not less than 0.1% and not more than 2.75%, not less than 0.1% and not more than 2.5%, not less than 0.1% and not more than 2.25%, not less than 0.1% and not more than 2%, not less than 0.1% and not more than 1.75%, not less than 0.1% and not more than 1.5%, not less than 0.1% and not more than 1.25%, not less than 0.01% and not more than 1%, not less than 0.01% and not more than 0.9%, not less than 0.01% and not more than 0.8%, not less than 0.01% and not more than 0.7%, not less than 0.01% and not more than 0.6%, not less than 0.010% and not more than 0.5%, not less than 0.010% and not more than 0.4%, not less than 0.01% and not more than 0.3%, not less than 0.01% and not more than 0.25%, not less than 0.01% and not more than 0.2%, not less than 0.001% and not more than 0.15%, not less than 0.001% and not more than 0.1%, not less than 0.001% and not more than 0.09%, not less than 0.001% and not more than 0.08%, not less than 0.001% and not more than 0.07%, not less than 0.001% and not more than 0.06%, not less than 0.001% and not more than 0.05%, not less than 0.001% and not more than 0.04%, not less than 0.001% and not more than 0.03%, not less than 0.001% and not more than 0.02%, or not less than 0.001% and not more than 0.01% (w/w) of the composition.

### Enteric-Resistant Delayed Release Endoxifen Compositions

The endoxifen compositions described herein may be formulated as enteric-resistant delayed release formulations for oral delivery. An enteric-resistant delayed release endoxifen formulation may be resistant to dissolution in the acidic environment of the stomach following oral administration, slowing or preventing release of endoxifen in the stomach, and may readily dissolve in the less acidic environment of the intestines, releasing most of the endoxifen in the intestines. The enteric-resistant delayed release formulations of the present disclosure may facilitate delivery of (Z)-endoxifen by protecting the (Z)-endoxifen from the acidic environment of the stomach and preventing isomerization into (E)-endoxifen.

Enteric-resistant and delayed release properties of an endoxifen composition may be assessed using a dissolution assay, which may be performed using a variety of techniques. In some embodiments, dissolution properties of an endoxifen composition may be quantified using the dissolution assay described in TABLE 18 of EXAMPLE 16. An endoxifen composition, such as an endoxifen formulation encapsulated in an enteric-resistant delayed release capsule, may be placed in an acidic solution comprising a pH of less than about 2 for about 120 minutes, stirring at a temperature of about 37±0.5° C. Dissolution rates under acidic conditions, mimicking the stomach environment, may be assessed by measuring the concentration of endoxifen in the solution at multiple timepoints (e.g., at 30 minutes, 60 minutes, 90 minutes, 120 minutes, or combinations thereof). After about 120 minutes, the acidic solution may be replaced with a buffered solution comprising a pH of about 6.8 and about 0.75% Polysorbate 80 and stirred for about 90 minutes at a temperature of about 37±0.5° C. Dissolution rates under less acidic conditions, mimicking the intestinal environment, may be assessed by measuring the concentration of endoxifen in the solution at multiple timepoints (e.g., at 15 minutes, 30 minutes, 45 minutes, 60 minutes, 90 minutes, or combinations thereof). Alternatively, dissolution rates may be measured using a US pharmacopoeia (USP) dissolution method or a Japanese pharmacopoeia (JP) dissolution method.

An enteric-resistant delayed release formulation may release no more than about 1%, no more than about 2%, no more than about 3%, no more than about 4%, no more than about 5%, no more than about 6%, no more than about 7%, no more than about 8%, no more than about 9%, no more than about 10%, no more than about 15%, no more than about 20%, no more than about 25%, no more than about 30%, no more than about 35%, or no more than about 40% of (Z)-endoxifen in the composition within about 2 hours in an acidic solution comprising a pH of less than about 2. In some embodiments, an enteric-resistant delayed release formulation may release from about 0% to about 1%, from about 0% to about 2%, from about 0% to about 3%, from about 0% to about 4%, from about 0% to about 5%, from about 0% to about 10%, from about 0% to about 15%, from about 0% to about 20%, from about 1% to about 2%, from about 1% to about 3%, from about 1% to about 4%, from about 1% to about 5%, from about 1% to about 10%, from about 1% to about 15%, from about 1% to about 20%, from about 3% to about 3%, from about 3% to about 4%, from about 3% to about 5%, from about 3% to about 10%, from about 3% to about 15%, or from about 3% to about 20% of (Z)-endoxifen in the composition within about 2 hours in an acidic solution comprising a pH of less than about 2.

In some embodiments, an enteric-resistant delayed release formulation may release no more than about 1%, no more than about 2%, no more than about 3%, no more than about 4%, no more than about 5%, no more than about 6%, no more than about 7%, no more than about 8%, no more than about 9%, no more than about 10%, no more than about 15%, no more than about 20%, no more than about 25%, no more than about 30%, no more than about 35%, or no more than about 40% of (Z)-endoxifen in the composition within about 2 hours under acidic conditions, measured as described in TABLE 18 of EXAMPLE 16. In some embodiments, an enteric-resistant delayed release formulation may release from about 0% to about 1%, from about 0% to about 2%, from about 0% to about 3%, from about 0% to about 4%, from about 0% to about 5%, from about 0% to about 10%, from about 0% to about 15%, from about 0% to about 20%, from about 1% to about 2%, from about 1% to about 3%, from about 1% to about 4%, from about 1% to about 5%, from about 1% to about 10%, from about 1% to about 15%, from about 1% to about 20%, from about 3% to about 3%, from about 3% to about 4%, from about 3% to about 5%, from about 3% to about 10%, from about 3% to about 15%, or from about 3% to about 20% of (Z)-endoxifen in the composition within about 2 hours under acidic conditions, measured as described in TABLE 18 of EXAMPLE 16.

An enteric-resistant delayed release formulation may release at least about 50%, at least about 55%, at least about 60%, at least about 65%, at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% of (Z)-endoxifen in the composition within about 1.5 hours in a solution comprising a pH of about 6.8 at about 0.75% polysorbate 80. In some embodiments, an enteric-resistant delayed release formulation may release from about 50% to about 95%, from about 60% to about 95%, from about 70% to about 95%, from about 80% to about 95%, from about 85% to about 95%, from about 86% to about 95%, from about 87% to about 95%, from about 88% to about 95%, from about 89% to about 95%, from about 90% to about 95%, from about 91% to about 95%, from about 92% to about 95%, from about 93% to about 95%, from about 94% to about 95%, from about 50% to about 97%, from about 60% to about 97%, from about 70% to about 97%, from about 80% to about 97%, from about 85% to about 97%, from about 86% to about 97%, from about 87% to about 97%, from about 88% to about 97%, from about 89% to about 97%, from about 90% to about 97%, from about 91% to about 97%, from about 92% to about 97%, from about 93% to about 97%, from about 94% to about 97%, from about 95% to about 97%, from about 50% to about 99%, from about 60% to about 99%, from about 70% to about 99%, from about 80% to about 99%, from about 85% to about 99%, from about 86% to about 99%, from about 87% to about 99%, from about 88% to about 99%, from about 89% to about 99%, from about 90% to about 99%, from about 91% to about 99%, from about 92% to about 99%, from about 93% to about 99%, from about 94% to about 99%, from about 95% to about 99%, from about 50% to about 100%, from about 60% to about 100%, from about 70% to about 100%, from about 80% to about 100%, from about 85% to about 100%, from about 86% to about 100%, from about 87% to about 100%, from about 88% to about 100%, from about 89% to about 100%, from about 90% to about 100%, from about 91% to about 100%, from about 92% to about 100%, from about 93% to about 100%, from about 94% to about 100%, or from about 95% to about 100% of (Z)-endoxifen in the composition within about 1.5 hours in a solution comprising a pH of about 6.8 at about 0.75% polysorbate 80.

In some embodiments, an enteric-resistant delayed release formulation may release at least about 50%, at least about 55%, at least about 60%, at least about 65%, at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% of (Z)-endoxifen in the composition within about 1.5 hours in a solution comprising a pH of about 6.8 at about 0.75% polysorbate 80, measured as described in TABLE 18 of EXAMPLE 16. In some embodiments, an enteric-resistant delayed release formulation may release from about 50% to about 95%, from about 60% to about 95%, from about 70% to about 95%, from about 80% to about 95%, from about 85% to about 95%, from about 86% to about 95%, from about 87% to about 95%, from about 88% to about 95%, from about 89% to about 95%, from about 90% to about 95%, from about 91% to about 95%, from about 92% to about 95%, from about 93% to about 95%, from about 94% to about 95%, from about 50% to about 97%, from about 60% to about 97%, from about 70% to about 97%, from about 80% to about 97%, from about 85% to about 97%, from about 86% to about 97%, from about 87% to about 97%, from about 88% to about 97%, from about 89% to about 97%, from about 90% to about 97%, from about 91% to about 97%, from about 92% to about 97%, from about 93% to about 97%, from about 94% to about 97%, from about 95% to about 97%, from about 50% to about 99%, from about 60% to about 99%, from about 70% to about 99%, from about 80% to about 99%, from about 85% to about 99%, from about 86% to about 99%, from about 87% to about 99%, from about 88% to about 99%, from about 89% to about 99%, from about 90% to about 99%, from about 91% to about 99%, from about 92% to about 99%, from about 93% to about 99%, from about 94% to about 99%, from about 95% to about 99%, from about 50% to about 100%, from about 60% to about 100%, from about 70% to about 100%, from about 80% to about 100%, from about 85% to about 100%, from about 86% to about 100%, from about 87% to about 100%, from about 88% to about 100%, from about 89% to about 100%, from about 90% to about 100%, from about 91% to about 100%, from about 92% to about 100%, from about 93% to about 100%, from about 94% to about 100%, or from about 95% to about 100% of (Z)-endoxifen in the composition within about 1.5 hours in a solution comprising a pH of about 6.8 at about 0.75% polysorbate 80, measured as described in TABLE 18 of EXAMPLE 16.

In some embodiments, an endoxifen formulation (e.g., a high dose oral endoxifen formulation) is in the form of solid dosage forms such as capsules, tablets, mini-tablets, beads, microbeads, granules, spheres particles, multi-particulates, and the like. The endoxifen compositions of the present disclosure can be formulated to target release in the intestines and colon. Accordingly, in some embodiments, the endoxifen compositions are in the form of enteric-resistant delayed release capsules, enteric coated delayed release tablets, enteric coated delayed release tablet-in-tablets, enteric coated delayed release tablet-in-capsules, beads-in-capsules, spheres-in capsules, and the like. For example, an endoxifen formulation may be encapsulated in an enteric-resistant delayed release capsule, such as a hydroxypropyl methylcellulose (also referred to as HPMC or Hypromellose) capsule to form an enteric-resistant delayed release endoxifen composition. In some embodiments, endoxifen (e.g., (Z)-endoxifen) may be dispersed in the endoxifen compositions homogeneously.

An enteric-resistant delayed release capsule, also referred to as an enteric-resistant delayed release capsule, may encapsulate an endoxifen formulation of the present disclosure (e.g., a high dose endoxifen formulation). In some embodiments, an enteric-resistant delayed release capsule may comprise hydroxypropyl methylcellulose, gellan gum, gelatin, hydroxypropyl methylcellulose phthalate, a coloring agent, an opacifier, or any combination thereof. An enteric-resistant delayed release capsule may comprise at least about 50%, at least about 55%, at least about 60%, at least about 65%, at least about 70%, at least about 75%, at least about 80%, at least about 85%, at least about 90%, or at least about 95% hydroxypropyl methylcellulose by weight, relative to total capsule weight (e.g., not including the endoxifen formulation filling). In some embodiments, an enteric-resistant delayed release capsule may comprise from about 50% to about 99%, from about 55% to about 99%, from about 60% to about 99%, from about 65% to about 99%, from about 70% to about 99%, from about 75% to about 99%, from about 80% to about 99%, from about 85% to about 99%, from about 50% to about 95%, from about 55% to about 95%, from about 60% to about 95%, from about 65% to about 95%, from about 70% to about 95%, from about 75% to about 95%, from about 80% to about 95%, from about 85% to about 95%, from about 50% to about 90%, from about 55% to about 90%, from about 60% to about 90%, from about 65% to about 90%, from about 70% to about 90%, from about 75% to about 90%, from about 80% to about 90%, or from about 85% to about 90% hydroxypropyl methylcellulose by weight, relative to total capsule weight.

An enteric-resistant delayed release capsule may comprise at least about 1%, at least about 2%, at least about 3%, at least about 4%, at least about 5%, at least about 6%, at least about 7%, at least about 8%, at least about 9%, or at least about 10% gellan gum, gelatin, or a combination thereof by weight, relative to total capsule weight. In some embodiments, an enteric-resistant delayed release capsule may comprise from about 1% to about 10%, from about 2% to about 10%, from about 3% to about 10%, from about 4% to about 10%, from about 5% to about 10%, from about 1% to about 9%, from about 2% to about 9%, from about 3% to about 9%, from about 4% to about 9%, from about 5% to about 9%, from about 1% to about 8%, from about 2% to about 8%, from about 3% to about 8%, from about 4% to about 8%, from about 5% to about 8%, from about 1% to about 7%, from about 2% to about 7%, from about 3% to about 7%, from about 4% to about 7%, from about 5% to about 7%, from about 1% to about 6%, from about 2% to about 6%, from about 3% to about 6%, from about 4% to about 6%, from about 5% to about 6%, from about 1% to about 5%, from about 2% to about 5%, from about 3% to about 5%, or from about 4% to about 5% gellan gum, gelatin, or a combination thereof by weight, relative to total capsule weight.

Plasticizers may be added to control the softness or pliability of oral dosage forms such as shell of a capsule, caplet, or a tablet and thus, may improve the mechanical properties of the pH-sensitive materials of the capsules or coatings on the oral dosage forms. Suitable plasticizers, include, without limitation, petroleum oils (for e.g., a paraffinic process oil, a naphthenic process oil, and an aromatic process oil), squalene, squalane, plant oils, (e.g., olive oil, camelia oil, castor oil, tall oil, and a peanut oil), silicon oils, dibasic acid esters, (e.g., dibutyl phthalate, and dioctyl phthalate), liquid rubbers (e.g., polybutene and a liquid isoprene rubber), liquid fatty acid esters (e.g., isopropyl myristate ISM), hexyl laurate, diethyl sebacate, and diisopropyl sebacate, triethyl citrate, triacetin, diethylene glycol, polyethylene glycols, polypropylene glycol, phthalates, sorbitol, glycol salicylate, crotaminton, and glycerin or mixtures thereof. The amount of plasticizer may vary depending upon the chemical composition of the pharmaceutical preparation. In one embodiment, the at least one plasticizer is sorbitol, dimethyl isosorbide, or a glycerol. In another embodiment, the plasticizer is 1% to 10%, such as 3% to 5% (wt/wt), of the composition.

Examples of glidants include, but are not limited to, colloidal silicone dioxide, cellulose, calcium phosphate, di or tri-basic and the like.

Compositions formulated for oral delivery as disclosed herein, for example, tablets, caplets, and capsules, may be coated with one or more enteric coating agent, control release agent or film forming agent to control or delay disintegration and absorption of the compositions comprising endoxifen or salts thereof in the gastrointestinal tract and thereby provide a sustained action over a longer period of time. Accordingly, in some embodiments, the tablet can be an enteric tablet, the caplet can be an enteric caplet, or the capsule can be an enteric capsule. The enteric tablets, enteric caplets, or enteric capsules of the present disclosure may be prepared by techniques known in the art.

Pharmaceutical preparations disclosed herein may comprise a control release agent. Examples of control release agent suitable for use include, without limitation, pH-dependent polymers, acid-insoluble polymers, methyl acrylate-methacrylic acid copolymers, cellulose acetate phthalate (CAP), cellulose acetate succinate, hydroxypropyl methyl cellulose phthalate, hydroxypropyl methyl cellulose acetate succinate (hypromellose acetate succinate), polyvinyl acetate phthalate (PVAP), methyl methacrylate-methacrylic acid copolymers, shellac, cellulose acetate trimellitate, sodium alginate, zein, waxes, including synthetic waxes, microcrystalline waxes, paraffin wax, carnauba wax, and beeswax; polyethoxylated castor oil derivatives, hydrogenated oils, glyceryl mono-, di-tribenates, glyceryl monostearate, glyceryl distearate, long chain alcohols, such as stearyl alcohol, cetyl alcohol, and polyethylene glycol; and mixtures thereof. In some embodiments, a time delay material such as glyceryl monostearate or glyceryl distearate may be used. In other embodiments, the controlled release reagent is a digestible waxy substance such as hard paraffin wax.

In some embodiments, a pharmaceutical composition comprising endoxifen (e.g., Z-endoxifen) may be formulated as a sustained release composition. In some embodiments, a pharmaceutical composition comprising a phosphoinositide 3-kinase inhibitor (e.g., alpelisib) may be formulated as a sustained release composition. Sustained release agent present in a sustained release composition of the present disclosure may be any sustained release agent known in the art to slow the release of a hydrophobic drug such as (Z)-endoxifen or a polymorph or a salt thereof.

Examples of sustained release agents include cellulosic ethers, gums, acrylic resins such as polymers and copolymers of acrylic acid, methacrylic acid, methyl acrylate, methyl methylacrylate, and combinations thereof, polyvinyl pyrrolidine, and protein-derived compounds. Examples of cellulosic ethers include hydroxyalkyl celluloses, hydroxyethyl celluloses, hydroxypropyl celluloses, hydroxypropyl methylcelluloses (HPMC or Hypromellose, for example Nos. 2208, 2906, 2910), hydroxypropyl methylcellulose phthalate (HPMCP or Hypromellose phthalate), carboxyalkyl celluloses, and carboxymethyl celluloses. In some embodiments, the at least one sustained release agent is a pH sustained release agent such as acid insoluble polymers which become increasingly soluble and permeable above pH 5.0 but remaining impermeable below pH 5.0. Such controlled release polymers target upper small intestines and/or colon. Non-limiting examples of acid-insoluble polymers include cellulose acetate phthalate, cellulose acetate butyrate, hydroxypropyl methyl cellulose phthalate, algenic acid salts such as sodium or potassium alginate, shellac, pectin, acrylic acid-methylacrylic acid copolymers, including those available commercially from EVONIK® or ROHM® (e.g., EUDRAGIT® sustained release polymers EUDRAGIT® RL (high permeability), EUDRAGIT® RS (low permeability) and EUDRAGIT® NM 30D (low permeability))—alone or in any combination thereof to achieve the desired permeability for sustained release.

In some embodiments, compositions may comprise one or more of pH-dependent polymers such as acid insoluble polymers. The pH-dependent polymers become increasingly permeable above pH 5.0 but are impermeable at pH below 5.0 whereas acid insoluble polymers become soluble in neutral to weakly alkaline conditions. Such control release polymers target upper small intestines and colon. Non-limiting examples of acid-insoluble polymers include cellulose acetate phthalate, cellulose acetate butyrate, hydroxypropyl methyl cellulose phthalate, algenic acid salts such as sodium or potassium alginate, shellac, pectin, acrylic acid-methylacrylic acid copolymers (commercially available under the tradename EUDRAGIT® L and EUDRAGIT® S from Rohm America Inc., Piscataway, NJ as a powder or a 30% aqueous dispersion; or under the tradename EASTACRYL®, from Eastman Chemical Co., Kingsport, TN, as a 30% dispersion). Additional examples include EUDRAGIT® L100-55, EUDRAGIT® L30D-55, EUDRAGIT® L100, EUDRAGIT® L100 12,5, EUDRAGIT® S100, EUDRAGIT® S12,5, EUDRAGIT® FS 30D, EUDRAGIT® E100, EUDRAGIT® E 12,5, and EUDRAGIT® PO. In at least one embodiment, the composition comprises EUDRAGIT® L100-55. EUDRAGIT® RS and RL and EUDRAGIT® NE and NM are also useful polymers for the purpose of this disclosure. In some embodiments, the composition comprises EUDRAGIT® L30D 55. In another embodiment, the preparation comprises EUDRAGIT® FS 30D. One of skill in the art will recognize that at least some acid insoluble polymers listed herein will also be biodegradable.

Commercially available delayed release capsules such as those available from Capsugel (e.g., VCAPS® Plus enteric capsules), can be used to prepared enteric-resistant delayed release capsules and are encompassed in the present disclosure. In some embodiments, the enteric delayed release capsules can be non-animal-based capsules, such as a Hypromellose capsule (for example, commercially available self-gelling VCAPS®, VCAPS® Plus, VCAPS® Enteric, other enteric capsules made using XCELLODOSE®, DRCAPS®, Encap Colonic Delivery (ENCODE), and ENTRINSIC™ drug delivery technology from CAPSUGEL®). Other technologies known in the art and available commercially (for example, QUALICAPS®, USA, NutraScience, USA, etc.) for the formulating enteric forms of oral solid dosage forms can also be utilized. For example, the endoxifen formulations of the present disclosure may be encapsulated in a DRCAPS® enteric-resistant delayed release capsule. In at least one embodiment, the capsule is an API-in-capsule, meaning that the (Z)-endoxifen free base or salts thereof is filled neat into the capsule. In such API-in-capsule oral dosage forms, the active ingredient, (Z)-endoxifen or salts thereof can be free flowing powders or micronized powders. When the dosage form is a capsule, in at least one embodiment, the capsule can be a seamless capsule or a banded capsule.

An oral dosage form can be of any shape suitable for oral administration, such as spherical (0.05-5 mL), oval (0.05-7 mL), ellipsoidal, pear (0.3-5 mL), cylindrical, cubic, regular and/or irregular shaped. An oral dosage form may be of any size suitable for oral administration, for example, size 0, size 2, and the like.

An example of an enteric-resistant delayed release endoxifen composition may comprise an enteric-resistant delayed release capsule comprising from about from about 65% to about 95% hydroxypropyl methylcellulose by weight and from about 3% to about 10% gellan gum by weight, relative to total unfilled capsule weight, encapsulating an endoxifen formulation comprising from about 10% to about 30% (Z)-endoxifen by weight, from about 1% to about 5% croscarmellose sodium by weight, from about 60% to about 95% microcrystalline cellulose by weight, and from about 0.5% to about 3% magnesium stearate by weight, relative to total fill weight of the composition. In some embodiments, the enteric-resistant delayed release endoxifen composition may be formulated in a dosage form comprising about 10 mg, about 20 mg, about 40 mg, or about 80 mg of (Z)-endoxifen per capsule.

### Oral Compositions

In some embodiments, the present disclosure relates to a composition formulated for oral administration comprising: 1 mg to 200 mg of endoxifen (e.g., Compound S3 or Compound E1), or a pharmaceutically acceptable salt, solvate, or tautomer thereof, and one or more compounds selected from TABLE 1 (e.g., Compound 1, Compound 2, Compound 3, Compound 4a, Compound 4b, Compound 5a, Compound 5b, Compound 6a, Compound 6b, Compound 7a, Compound 7b, Compound 8, or a pharmaceutically acceptable salt, solvate, or tautomer thereof) per unit dose. In some aspects, the composition formulated for oral administration comprises from 1 mg to 80 mg per unit dose, from 2 mg to 80 mg per unit dose, from 4 mg to 80 mg per unit dose, or from 4 mg to 40 mg per unit dose, of endoxifen (e.g., Compound S3 or Compound E1), or a pharmaceutically acceptable salt, solvate, or tautomer thereof, and one or more endoxifen-related compounds (e.g., Compound 1, Compound 2, Compound 3, Compound 4a, Compound 4b, Compound 5a, Compound 5b, Compound 6a, Compound 6b, Compound 7a, Compound 7b, Compound 8, or a pharmaceutically acceptable salt, solvate, or tautomer thereof). In some aspects, the composition formulated for oral administration comprises about 1 mg, about 2 mg, about 4 mg, about 6 mg, about 8 mg, about 10 mg, about 20 mg, about 40 mg, or about 80 mg per unit dose of endoxifen (e.g., Compound S3 or Compound E1), or a pharmaceutically acceptable salt, solvate, or tautomer thereof, and one or more endoxifen-related compounds (e.g., Compound 1, Compound 2, Compound 3, Compound 4a, Compound 4b, Compound 5a, Compound 5b, Compound 6a, Compound 6b, Compound 7a, Compound 7b, Compound 8, or a pharmaceutically acceptable salt, solvate, or tautomer thereof).

In a still further aspect, the present disclosure relates to compositions comprising endoxifen (e.g., Compound S3 or Compound E1) wherein the composition further comprises 0.1% to 10% (wt/wt) of one or more endoxifen-related compounds (e.g., Compound 1, Compound 2, Compound 3, Compound 4a, Compound 4b, Compound 5a, Compound 5b, Compound 6a, Compound 6b, Compound 7a, Compound 7b, Compound 8, or a pharmaceutically acceptable salt, solvate, or tautomer thereof). In some cases, a composition comprising endoxifen (e.g., Compound S3 or Compound E1) and one of more endoxifen-related compounds (e.g., Compound 1, Compound 2, Compound 3, Compound 4a, Compound 4b, Compound 5a, Compound 5b, Compound 6a, Compound 6b, Compound 7a, Compound 7b, Compound 8, or a pharmaceutically acceptable salt, solvate, or tautomer thereof) is a solid, a powder, a liquid, a suspension, or an emulsion. A solid comprising one or more compounds selected from TABLE 1 of a composition disclosed herein can be amorphous, polycrystalline, or of a single crystalline form. In some embodiments, a pharmaceutical composition comprising endoxifen (e.g., Compound S3 or Compound E1) and one of more endoxifen-related compounds (e.g., Compound 1, Compound 2, Compound 3, Compound 4a, Compound 4b, Compound 5a, Compound 5b, Compound 6a, Compound 6b, Compound 7a, Compound 7b, Compound 8, or a pharmaceutically acceptable salt, solvate, or tautomer thereof), may further comprise a liquid. In some embodiments, a composition comprising endoxifen (e.g., Compound S3 or Compound E1) and one of more endoxifen-related compounds (e.g., Compound 1, Compound 2, Compound 3, Compound 4a, Compound 4b, Compound 5a, Compound 5b, Compound 6a, Compound 6b, Compound 7a, Compound 7b, Compound 8, or a pharmaceutically acceptable salt, solvate, or tautomer thereof), may be suspended in the liquid. Liquids for suspension of a composition comprising endoxifen (e.g., Compound S3 or Compound E1) and one of more endoxifen-related compounds (e.g., Compound 1, Compound 2, Compound 3, Compound 4a, Compound 4b, Compound 5a, Compound 5b, Compound 6a, Compound 6b, Compound 7a, Compound 7b, Compound 8, or a pharmaceutically acceptable salt, solvate, or tautomer thereof), may comprise water, an alcohol, an organic solvent, a pharmaceutically acceptable diluent, a pharmaceutically acceptable liquid or solvent, or an aqueous solution.

In another aspect, the present disclosure relates to a composition comprising endoxifen (e.g., Compound S3 or Compound E1) and one of more endoxifen-related compounds (e.g., Compound 1, Compound 2, Compound 3, Compound 4a, Compound 4b, Compound 5a, Compound 5b, Compound 6a, Compound 6b, Compound 7a, Compound 7b, Compound 8, or a pharmaceutically acceptable salt, solvate, or tautomer thereof) further comprising one or more excipients.

In one aspect, the present disclosure relates to a composition comprising endoxifen (e.g., Compound S3 or Compound E1) and one of more endoxifen-related compounds (e.g., Compound 1, Compound 2, Compound 3, Compound 4a, Compound 4b, Compound 5a, Compound 5b, Compound 6a, Compound 6b, Compound 7a, Compound 7b, Compound 8, or a pharmaceutically acceptable salt, solvate, or tautomer thereof), wherein the composition is formulated for oral, parenteral, topical, and intraductal delivery.

In another aspect, the present disclosure relates to a composition comprising endoxifen (e.g., Compound S3 or Compound E1) and one of more endoxifen-related compounds (e.g., Compound 1, Compound 2, Compound 3, Compound 4a, Compound 4b, Compound 5a, Compound 5b, Compound 6a, Compound 6b, Compound 7a, Compound 7b, Compound 8, or a pharmaceutically acceptable salt, solvate, or tautomer thereof), wherein the composition formulated for oral delivery is a tablet, a caplet, a capsule, a pill, a powder, a troche, an elixir, a suspension, a syrup, a wafer, a chewing gum, a dragee, and a lozenge.

### Methods of Making Endoxifen Compositions

Processes for preparing the endoxifen composition described herein are provided as further embodiments of the present disclosure and are illustrated by the following procedures in which the meanings of the generic compounds (e.g., Compound S1, Compound S2, Compound S3, Compound E1, Compound 1, Compound 2, Compound 3, Compound 4a, Compound 4b, Compound 5a, Compound 5b, Compound 6a, Compound 6b, Compound 7a, Compound 7b, and Compound 8) are as given above unless otherwise qualified.

In various aspects, compounds of the present disclosure, including those of Compound S1, Compound S2, Compound S3, Compound E1, Compound 1, Compound 2, Compound 3, Compound 4a, Compound 4b, Compound 5a, Compound 5b, Compound 6a, Compound 6b, Compound 7a, Compound 7b, and Compound 8, can be produced according to SCHEME 1 below.

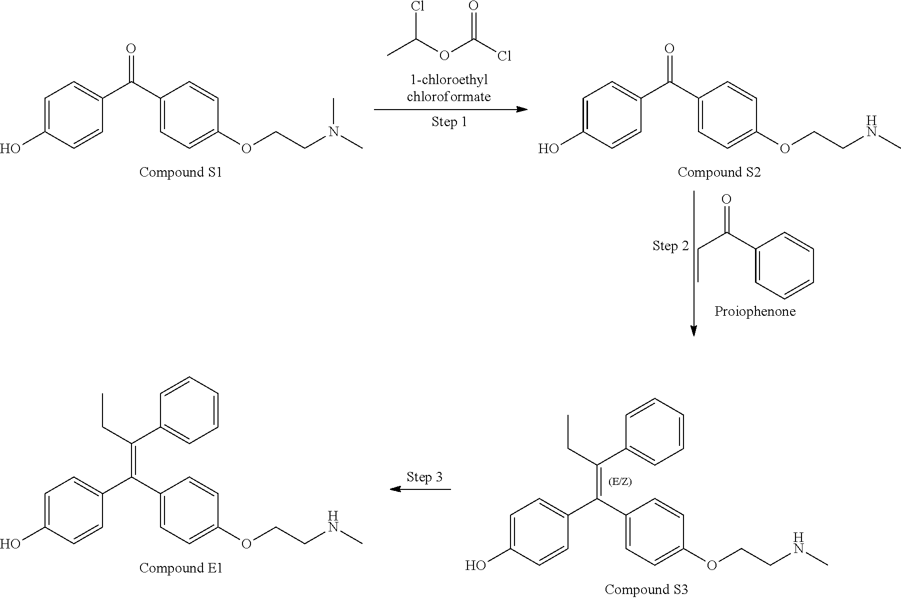

As illustrated in SCHEME 1, the endoxifen compositions of the present disclosure may be prepared by a synthetic scheme comprising three steps. Step 1 of the synthetic scheme comprises a demethylation reaction, Step 2 of the synthetic scheme comprises a coupling reaction (e.g., a McMurry Reaction), and Step 3 of the synthetic scheme comprises an enrichment method. Each of the steps is further generally described below.

### Step 1—Demethylation

As illustrated in SCHEME 1, the synthetic scheme for the endoxifen compositions described herein may comprise demethylating a Compound S1 to form Compound S2. Step 1 may also produce Compound 1, Compound 2, and Compound 3. The demethylation of Compound S1 in Step 1 of SCHEME 1 may occur by reaction Compound S1 with a compound capable of accepting a methyl group (e.g., a demethylating agent) to form Compound S2. In some embodiments, Compound S2 may also further react with one or more of the reaction components. Side reactions of Compound S2 may form Compound 1 or Compound 2. Compound S1 may also undergo a side reaction to become methylated to form Compound 3.

In some embodiments, Compound S1 may be demethylated by a demethylating agent. In some embodiments, the demethylating agent may be 1-chloroethyl chloroformate. In certain embodiments, the demethylating comprises 1 to 1.5, 1 to 2, 1 to 2.5, 1 to 3, 1 to 4, 1 to 5, 1.5 to 2, 1.5 to 2.5, 1.5 to 3, 1.5 to 4, 1.5 to 5, 2 to 2.5, 2 to 3, 2 to 4, 2 to 5, 2.5 to 3, 2.5 to 4, 2.5 to 5, 3 to 4, 3 to 5, or 4 to 5 equivalents of the demethylating agent relative to the Compound S1.

In some embodiments, the demethylation comprises an organic solvent. In some embodiments, the organic solvent comprises a solvent mixture. In some embodiments, the solvent mixture comprises tetrahydrofuran and N-ethyldiisopropylamine. In some embodiments, the organic solvent is dichloromethane, dichloroethane, chloroform, carbon tetrachloride, chlorobenzene, diethyl ether, 1,4-dioxane, tert-butyl methyl ether (TBME), tetrahydrofuran (THF), N,N-dimethylformamide (DMF), N-methylpyrrolidone (NMP), diglyme, nitromethane, 1,2-dimethoxyethane (DME), pyridine, acetone, acetonitrile, benzene, o-xylene, m-xylene, p-xylene, xylenes, hexanes, cyclohexane, heptane, octane, nonane, and decane, or a combination thereof. In some aspects, the organic solvent comprises a base. In certain aspects, the base is a tertiary amine. In particular aspects, the base is ehtyldiisopropylamine. In particular aspects, the demethylating comprises between 1 and 2, 1 and 3, 1 and 4, 1 and 5, 1 and 6, 1 and 8, 2 and 3, 2 and 4, 2 and 5, 2 and 6, 2 and 8, 3 and 4, 3 and 5, 3 and 6, 3 and 8, 4 and 5, 4 and 6, 4 and 8, 5 and 6, 5 and 8, or 6 and 8 equivalents of the base relative to the Compound S1.

In some embodiments, the method further comprises incubating the demethylation under an inert atmosphere. In some embodiments, the inert atmosphere is a N₂ atmosphere. In some embodiments, the method further comprises heating the demethylation to between 30 to 70° C., between 40 to 70° C., between 50 to 70° C., or between 55 to 65°. In some embodiments, the method further comprises heating the coupling to an elevated temperature for between 8 to 16 hours, between 10 to 14 hours, or between 9 to 11 hours. In some embodiments, the elevated temperature is a boiling point of the organic solvent. In some embodiments, the method further comprises adding an alcohol to the demethylation. In some embodiments, the alcohol is methanol. In some embodiments, the alcohol is methanol, ethanol, propanol, butanol, pentanol, hexanol, heptanol, octanol, nonanol, decanol, undecanol, or dodecanol. In some embodiments, the alcohol is a cyclic alcohol (e.g., benzyl alcohol, a sterol, an inositol, cyclohexanol, methylcyclohexanol, dimethylcyclohexanol, or a terineol). In some embodiments, the method further comprises heating the demethylation to between 30 to 70° C., between 40 to 70° C., between 50 to 70° C., or between 55 to 65° C. and distilling the coupling to a reduced volume. In some embodiments, the reduced volume to total volume ratio is 1:3, 1:5, or 1:10. In some embodiments, the method further comprises adding an acid. In some embodiments, the acid comprises 2N to 12N of the acid. In some embodiments, the method further comprises heating the demethylation to between 30 to 70° C., between 40 to 70° C., between 50 to 70° C., or between 55 to 65° C. for between 2 to 20 hours, between 6 to 18 hours, or between 8 to 16 hours. In some embodiments, the method further comprises distilling, washing, purifying, or separating Compound S2 using an acid, a base, an aqueous solution, an alcohol, or an organic solvent. In some embodiments, the aqueous solution is water. In some embodiments, the base comprises 2N to 12N of the base. In some embodiments, the base is sodium hydroxide. In some embodiments, the organic solvent is ethyl acetate. In some embodiments, the organic solvent is dichloromethane, dichloroethane, chloroform, carbon tetrachloride, chlorobenzene, diethyl ether, 1,4-dioxane, tert-butyl methyl ether (TBME), tetrahydrofuran (THF), N,N-dimethylformamide (DMF), N-methylpyrrolidone (NMP), diglyme, nitromethane, 1,2-dimethoxyethane (DME), pyridine, acetone, acetonitrile, benzene, o-xylene, m-xylene, p-xylene, xylenes, hexanes, cyclohexane, heptane, octane, nonane, and decane, or a combination thereof. In some embodiments, the acid comprises 2N to 12N of the acid. In some embodiments, the acid is hydrochloric acid.

In some aspects, the separating comprises phase separation. In some aspects, the separating comprises distillation. In particular aspects, the distillation comprises vacuum distillation. In further aspects, the distillation is performed at atmospheric pressure. In additional aspects, the distillation is performed at a temperature of between 5° C. and 10° C., 5° C. and 15° C., 5° C. and 20° C., 5° C. and 25° C., 5° C. and 30° C., 5° C. and 40° C., 5° C. and 50° C., 10° C. and 15° C., 10° C. and 20° C., 10° C. and 25° C., 10° C. and 30° C., 10° C. and 40° C., 10° C. and 50° C., 15° C. and 20° C., 15° C. and 25° C., 15° C. and 30° C., 15° C. and 40° C., 15° C. and 50° C., 20° C. and 25° C., 20° C. and 30° C., 20° C. and 35° C., 20° C. and 40° C., 20° C. and 50° C., 25° C. and 30° C., 25° C. and 40° C., 25° C. and 50° C., 30° C. and 40° C., 30° C. and 50° C., or 40° C. and 50° C.

In one aspect, the separating uses between 1 and 2, 1 and 3, 1 and 4, 1 and 5, 1 and 6, 1 and 8, 1 and 10, 1 and 12, 1.5 and 3, 1.5 and 4, 1.5 and 5, 1.5 and 6, 1.5 and 8, 1.5 and 10, 1.5 and 12, 2 and 3, 2 and 4, 2 and 5, 2 and 6, 2 and 8, 2 and 10, 2 and 12, 3 and 4, 3 and 5, 3 and 6, 3 and 8, 3 and 10, 3 and 12, 4 and 5, 4 and 6, 4 and 8, 4 and 10, 4 and 12, 5 and 6, 5 and 8, 5 and 10, 5 and 12, 6 and 8, 6 and 10, 6 and 12, 8 and 10, 8 and 12, or 10 and 12 equivalents of the acid (weight/weight). In another aspect, the separating uses between 3.2 and 4.8 equivalents of the acid (weight/weight). In a further aspect, the Compound S2 comprises less than 5%, less than 4%, less than 3%, less than 2.5%, less than 2%, less than 1.5%, less than 1%, less than 0.75%, less than 0.5%, less than 0.25%, or less than 0.1% Compound 8. In particular aspects, the Compound S2 exhibits less than 10%, less than 8%, less than 6%, less than 5%, less than 4%, less than 3%, less than 2%, less than 1.5%, or less than 1% weight loss upon drying. In further aspects, the Compound S2 comprises less than 5000 ppm, less than 4000 ppm, less than 3000 ppm, less than 2500 ppm, less than 2000 ppm, less than 1500 ppm, less than 1000 ppm, less than 500 ppm, less than 200 ppm, or less than 100 ppm DIPEA.

### Step 2—Carbonyl Coupling

As illustrated in SCHEME 1, a method of generating the endoxifen compositions as described herein may include coupling Compound S2 to propiophenone to form Compound S3. Side reactions in Step 2 of SCHEME 1 may also result in the formation of Compounds 4a and 4b, Compounds 5a and 5b, Compounds 6a and 6b. In some embodiments, the formation of Compounds 4a and 4b may result in specific isomeric preferences such as the (Z)-isomer (Compound 4b) or the (E)-isomer (Compound 4a). In some embodiments, the formation of Compounds 5a and 5b may result in specific isomeric preferences such as the (Z)-isomer (Compound 5a) or the (E)-isomer (Compound 5b). Compounds 4a and 4b may results from the homo-coupling of Compound S2. Compounds 5a and 5b may results from the coupling of Compound 3 and propiophenone. Compounds 6a and 6b may result from the homocoupling of propiophenone.

Coupling of Compound S2 to a propiophenone compound may be achieved through various olefination methods. The coupling can comprise reductively activating a carbonyl of Compound S2 and/or propiophenone. A reducing agent can be used to reduce the carbonyl to an organometallic intermediate, carbene, carbanion, or carbon radical. The carbonyl may be reduced via a one-electron reduction or a two-electron reduction.

The coupling can comprise a metal catalyst. Suitable metal catalysts for this coupling step include titanium, zirconium, vanadium, niobium, molybdenum, tungsten, aluminum, and zinc. The metal catalyst may be generated from a metal salt and a reducing agent such as zinc metal, Zn/Cu, LiAlH4, alkali and alkali earth metals, lithium arenes, and butyllithium.

In some cases, the coupling includes a McMurry reaction. As used herein, a McMurry reaction can denote a coupling reaction between two carbonyl functional groups to form a carbon-carbon double bond between the alpha-carbons of the carbonyl groups. The McMurry reaction is typically catalyzed by a reducing metal species such as low-valent titanium reagents which may be achieved by reacting titanium chlorides with a reducing agent. However, the McMurry reaction can utilize a non-titanium-based metal catalyst, such as zirconium, vanadium, niobium, molybdenum, tungsten, aluminum, and zinc. Carbonyl containing compounds may be coupled inter or intra molecularly by the McMurry reaction. The mechanism of the McMurry reaction may proceed through a pinacol intermediate and subsequent deoxygenation. The McMurry reaction can yield a mixture of the (E) and (Z) isomers, however the (E) isomer may be favored with sterically bulky substituents. Compound S3 may be prepared by a McMurry reaction via titanium salts such as chloride salts of titanium (for example, titanium trichloride (TiCl₃) and titanium tetrachloride (TiCl₄)) and reducing agents in inert organic solvents to form an E/Z mixture.

In some cases, the coupling utilizes a titanium, zirconium, vanadium, niobium, molybdenum, tungsten, aluminum, or zinc catalyst. In some cases, the catalyst is added at a wt/wt ratio of Compound S2 to titanium salt ranging from 1:0.1 to 1:12. For example, the reaction can include between 1 and 1.5, 1 and 2, 1 and 2.5, 1 and 3, 1 and 4, 1 and 5, 1 and 6, 1.5 and 2, 1.5 and 2.5, 1.5 and 3, 1.5 and 4, 1.5 and 5, 1.5 and 6, 2 and 2.5, 2 and 3, 2 and 4, 2 and 5, 2 and 6, 2.5 and 3, 2.5 and 4, 2.5 and 5, 2.5 and 6, 3 and 4, 3 and 5, 3 and 6, 4 and 5, 4 and 6, or 5 and 6 equivalents of the metal catalyst relative to the Compound S2 (weight/weight).

In some cases, the coupling utilizes a titanium catalyst. Titanium salts which may be used in the coupling include titanium halides (such as titanium trichloride (TiCl₃), titanium tetrachloride (TiCl₄), titanium iodides, titanium bromides, and titanium fluorides), titanium (IV) trichloride isopropoxide, and titanium isopropoxide. In some embodiments, the titanium salt is TiCl₄. Titanium salts, such as TiCl₄, can be added at a wt/wt ratio of Compound S2 to titanium salt ranging from 1:0.1 to 1:12.

Reducing agents which may be used in Step 2 of SCHEME 1 include zinc, zirconium, vanadium, niobium, molybdenum, tungsten, aluminum, magnesium, potassium, zinc-copper couple, alkali and alkali earth metals, butylium, lithium, and lithium aluminum hydride. The reducing agent may be capable of reducing the metal catalyst (e.g., converting Ti(IV) to Ti(III)). In at least one embodiment, the reducing agent is zinc. Step 2 of SCHEME 1 can be carried out using a reducing agent, such as zinc, at a wt/wt ratio of Compound S2 to reducing agent ranging from 1:0.1 to 1:10. In particular, the reaction can utilize between 2 and 3, 2 and 4, 2 and 5, 2 and 6, 2 and 8, 2 and 10, 3 and 4, 3 and 5, 3 and 6, 3 and 8, 3 and 10, 4 and 5, 4 and 6, 4 and 8, 4 and 10, 5 and 6, 5 and 8, 5 and 10, 6 and 8, 6 and 10, or 8 and 10 equivalents of the reducing agent relative to the Compound S2. In some embodiments, the ratio of reducing agent is in excess compared to titanium salts.

Step 2 of SCHEME 1 can be carried out in one or more organic solvents. Preferably, the organic solvent is an inert organic solvent. For example, the solvent can be inert to Compound S2, Compound S3, and coupling reagents such as the reducing agent and metal catalyst. Organic solvents useful for Step 2 of SCHEME 1 reaction include dichloromethane, dichloroethane, chloroform, carbon tetrachloride, chlorobenzene, diethyl ether, 1,4-dioxane, tert-butyl methyl ether (TBME), tetrahydrofuran (THF), N,N-dimethylformamide (DMF), N-methylpyrrolidone (NMP), diglyme, nitromethane, 1,2-dimethoxyethane (DME), pyridine, acetone, acetonitrile, benzene, o-xylene, m-xylene, p-xylene, xylenes, hexanes, cyclohexane, heptane, octane, nonane, and decane, or a combination thereof. In some embodiments, the organic solvent is at wt/wt ratio of Compound S2 to solvent ranging from 1:1 to 1:50. In other embodiments, the organic solvent is at wt/wt ratios of Compound S2 to solvent ranging from 1:1 to 1:20. In some embodiments, the organic solvent used for Step 2 of SCHEME 1 is THF. In some embodiments, the amount of THF used is a wt/wt ratio of Compound S2 to THF ranging from 1:1 to 1:20.

In some cases, in Step 2 of SCHEME 1, a metal catalyst (e.g., a titanium salt) and a reducing agent are combined in the organic solvent prior to the coupling reaction. In particular, it can be advantageous to combine the metal catalyst and the reducing agent in the organic solvent to create an activating mixture. The metal catalyst can be added to the reducing agent and organic solvent at such a rate so as to keep the internal temperature at most about 75° C., such as at most about 65° C., at most about 55° C., at most about 45° C., at most about 40° C., at most about 35° C., at most about 30° C., at most about 25° C., at most about 20° C., or at most about 15° C. Accordingly, the metal catalyst and the reducing agent in the organic solvent can be combined to create a pre-mix. In some embodiments, the metal catalyst is TiCl₄. In some embodiments the reducing agent is Zn. In some embodiments, the organic solvent is THF. In some embodiments, TiCl₄, Zn, and THF are combined to create TiCl₄/Zn/THF mixture prior to combining with Compound S2 and propiophenone. In at least one embodiment, TiCl₄ is added to Zn and THF at a temperature of at most about 20° C.

Preparation of the metal catalyst and the reducing agent in the organic solvent may further comprise heating the metal catalyst and the reducing agent in the organic solvent to a temperature ranging from about 20° C. to 250° C., such as from about 40° C. to 80° C., from about 50° C. to 230° C., from about 50° C. to 120° C., or from about 150° C. to 200° C. In some embodiments, the metal catalyst and the reducing agent present in the organic solvent may be heated to at least about 60° C. The metal catalyst and the reducing agent in the organic solvent may be heated to a temperature at least about 60° C. for a duration of at least about 30 min, such as at least about 1 hour, at least about 2 hours, at least about 4 hours, at least about 6 hours, or at least about 8 hours, under inert conditions such as under N₂ or argon.

It can be advantageous to pre-mix Compound S2 and propiophenone in an organic solvent before reacting them with the metal catalyst and the reducing agent. In some embodiments, the organic solvent is THF. Compound S2 and propiophenone can be combined in the organic solvent (e.g., THF) and then reacted with the pre-mixed metal catalyst/reducing agent/organic solvent mixture such as a TiCl₄/Zn/THF mixture to form Compound S3 (an (E/Z) mixture of endoxifen). Propiophenone can be added at a wt/wt ratio to Compound S2 ranging from about 1:0.01 to 1:5. The organic solvent can be added at a wt/wt ratio to Compound S2 ranging from about 1:1 to 1:20. Compound S2 can be reacted with the metal catalyst and reducing agent in the organic solvent at a temperature of at least about 60° C., for duration of at least about 0.5 hours, at least about 1 hour, at least about 2 hours, at least about 4 hours, at least about 6 hours, at least about 8 hours, at least about 12 hours, at least about 24 hours, or at least about 48 hours. In at least one embodiment, Compound S2 is mixed with THF and propiophenone and combined with a mixture of TiCl₄, Zn, THF and heated at a temperature of at least about 60° C. for a duration of at least about 8 hours. The reaction mixture may then be allowed to cool to a temperature range of about 0° C. to 35° C. In some embodiments, the product of the reaction mixture is primarily Compound S3.

Compound S3 produced in Step 2 of SCHEME 1 can be purified. In some cases, Compound S3 may be purified by extractive distillation, liquid-liquid extraction, crystallization, or a combination thereof. In some cases, Compound S3 may be purified by at least two of extractive distillation, liquid-liquid extraction, and crystallization. In some cases, Compound S3 may be purified by distillation, liquid-liquid extraction, and crystallization.

Compound S3 can be subject to extractive distillation by distillation with an organic solvent such as THF or MeTHF. In some embodiments, the organic solvent (e.g., MeTHF or THF) may be added at a vol/vol ratio to the distillate ranging from about 1:1 to 1:20. The distillation may be performed 2 or more times.

In some embodiments, after distillation, Compound S3 can be subject to liquid-liquid extraction. Compound S3 can be extracted from the Step 2 reaction mixture with acids or bases, such as sodium hydroxide or hydrochloric acid, extracted with organic solvents, such as THF and MeTHF, or extracted by addition of salts, such as potassium carbonate, ammonium chloride, sodium chloride, sodium hydroxide, to the reaction mixture and extraction with an organic solvent, such as THF and MeTHF.

In some embodiments, an acid, such as hydrochloric acid, is added to the reaction mixture and can be heated to at most about 65° C., at most about 55° C., at most about 50° C., at most about 45° C., at most about 40° C., at most about 35° C., at most about 30° C., at most about 25° C., or at most about 20° C. forming a biphasic reaction mixture with an aqueous phase and an organic phase. The biphasic reaction mixture can be stirred for at least about 0.5 hours, at least about 1 hour, or at least about 2 hours.

The biphasic reaction mixture may then be further extracted by addition of an organic solvent, such as MeTHF or THF and stirred at a temperature of at most about 65° C., at most about 55° C., at most about 50° C., at most about 45° C., at most about 40° C., at most about 35° C., at most about 30° C., at most about 25° C., or at most about 20° C. for a duration of at least about 0.5 hours, at least about 1 hour, or at least about 2 hours. The extraction by addition of an acidic solution (e.g., HCl) and organic solvent (e.g., MeTHF or THF) to the biphasic reaction mixture may then be repeated at least once, at least twice, at least three times, at least four times, or at least five times.

In some embodiments, the biphasic reaction mixture is extracted one or more times with a base. In some embodiments, the base is a carbonate, such as potassium carbonate (K₂CO₃), for example 40% K₂CO₃ (1:1 to 1:10 wt/wt) in MeTHF (1:1 to 1:10 wt/wt). In some embodiments, the biphasic reaction mixture may be further extracted with NaOH, such as TN NaOH (1:1 to 1:20 vol/vol). The biphasic reaction mixture can be stirred at a temperature of at most about 65° C., at most about 55° C., at most about 50° C., at most about 45° C., at most about 40° C., at most about 35° C., at most about 30° C., at most about 25° C., or at most about 20° C. for a duration of at least about 0.5 hours, at least about 1 hour, or at least about 2 hours. NaCl (1:0.1 to 1:1 wt/wt) may be added to biphasic reaction mixture for an extraction step and further stirred at a temperature of at most about 65° C., at most about 55° C., at most about 50° C., at most about 45° C., at most about 40° C., at most about 35° C., at most about 30° C., at most about 25° C., and at most about 20° C. for a duration of at least about 0.5 hours, at least about 1 hour, and at least about 2 hours.

From the liquid-liquid extraction, a solid precipitate can be obtained from the biphasic reaction mixture which can then be further washed with an organic solvent, such as MeTHF or THF (1:0.5 to 1:10 wt/wt). The precipitate can then be subject to further washing and extraction by dissolving the precipitate in a solution with NaOH and NaCl. The solution can be further extracted with NaOH addition, such as 1N NaOH (1:1 to 1:20 vol/vol). The solution can be stirred at a temperature of at most about 65° C., at most about 55° C., at most about 50° C., at most about 45° C., at most about 40° C., at most about 35° C., at most about 30° C., at most about 25° C., or at most about 20° C. for a duration of at least about 0.25 hours, at least about 0.5 hours, at least about 1 hour, or at least about 2 hours. In at least one embodiment, NaCl (1:0.1 to 1:1 wt/wt) may be added to the solution for an additional extraction step and further stirred at a temperature of at most about 65° C., at most about 55° C., at most about 50° C., at most about 45° C., at most about 40° C., at most about 35° C., at most about 30° C., at most about 25° C., or at most about 20° C. for a duration of at least about 0.5 hours, at least about 1 hour, or at least about 2 hours. The solution may then be left to separate at a temperature of at most about 65° C., at most about 55° C., at most about 50° C., at most about 45° C., at most about 40° C., at most about 35° C., at most about 30° C., at most about 25° C., or at most about 20° C. for a duration of at least about 0.5 hours, at least about 1 hour, or at least about 2 hours. The additional extraction step may be repeated at least once, at least twice, at least three times, at least four times, or at least five times. The organic phase of the biphasic solution can then be collected for further purification.

The organic phase may then be further washed with a NaCl solution ranging from about 10% to 20% NaCl and stirred at a temperature of at most about 65° C., at most about 55° C., at most about 50° C., at most about 45° C., at most about 40° C., at most about 35° C., at most about 30° C., at most about 25° C., and at most about 20° C. for a duration of at least about 0.5 hours, at least about 1 hour, and at least about 2 hours forming a biphasic solution. The biphasic solution may then be left to separate at a temperature of at most about 65° C., at most about 55° C., at most about 50° C., at most about 45° C., at most about 40° C., at most about 35° C., at most about 30° C., at most about 25° C., and at most about 20° C. for a duration of at least about 0.5 hours, at least about 1 hour, and at least about 2 hours. The organic phase of the biphasic solution can then be collected for further purification.

The organic phase can then be further distilled with an organic solvent, such as acetonitrile (ACN). In some embodiments, the ACN (1:0.5 to 1:10 vol/vol) is added to the distillate for further distillation. In some embodiments, the ACN distillation is performed once more or twice more resulting in a purified organic distillate.

The purified organic distillate can then be further distilled in a different organic solvent, such as acetone. In some embodiments the acetone (1:0.5 to 1:10 vol/vol) is added to the purified organic distillate and the resulting organic solution stirred at a temperature of at most about 65° C., at most about 55° C., at most about 50° C., at most about 45° C., at most about 40° C., at most about 35° C., at most about 30° C., at most about 25° C., or at most about 20° C. for a duration of at least about 0.5 hours, at least about 1 hour, or at least about 2 hours. The organic solution may then be allowed to cool to about 0° C. to 35° C. over a duration of at least about 2 hours, at least about 4 hours, and at least about 6 hours and further stirred at the cooled temperature for at least about 6 hours, at least about 8 hours, or at least about 10 hours. In some embodiments, after cooling the organic solution, a precipitate is present.

The precipitate can then be collected and washed with a pre-cooled solvent mixture of organic solvents, such as ACN/acetone (1:0.5 to 1:2 vol/vol). The washed precipitate can then be dried under vacuum at a temperature of at most about 85° C., at most about 80° C., at most about 75° C., at most about 70° C., at most about 65° C., or at most about 60° C. for a duration of at least about 2 hours, at least about 4 hours, or at least about 6 hours.

### Step 3—(Z)-Endoxifen Enrichment

As illustrated in SCHEME 1, a method of generating the endoxifen compositions as described herein may include enriching a composition of Compound S3 (mixture of (E/Z)-endoxifen) with a higher percentage of (Z)-endoxifen (Compound E1). In some embodiments, the enrichment results in >80%, >85%, >90%, >95%, >96%, >97%, >98%, >99%, >99.5% (Z)-endoxifen (Compound E1). In some embodiments, the method comprises purifying a composition of Compound S3 to comprise >90% of Compound E1. Side reactions during Step 3 may also result in the formation of Compounds 7a and 7b. Isomers may also be favored during the formation of Compounds 7 and 7b, such as the (Z)-isomer (Compound 7a) or the (E)-isomer (Compound 7b). Compounds 7a and 7b may form during Step 3 from acetylating Compound S3. Compound S3 may be acetylated on the amine group by a solvent used in Step 3 (e.g., ethyl acetate).

In some embodiments, the method of Step 3 of SCHEME 1, may be performed in an organic solvent. In some embodiments, the organic solvent comprises an aprotic solvent. In some embodiments, the organic solvent comprises ethyl acetate. In some embodiments, the organic solvent is dichloromethane, dichloroethane, chloroform, carbon tetrachloride, chlorobenzene, diethyl ether, 1,4-dioxane, tert-butyl methyl ether (TBME), tetrahydrofuran (THF), N,N-dimethylformamide (DMF), N-methylpyrrolidone (NMP), diglyme, nitromethane, 1,2-dimethoxyethane (DME), pyridine, acetone, acetonitrile, benzene, o-xylene, m-xylene, p-xylene, xylenes, hexanes, cyclohexane, heptane, octane, nonane, and decane, or a combination thereof. In some embodiments, the method further comprises the addition of an acid. In some embodiments, the acid comprises between about 2 to 12 N of the acid. In some embodiments, the acid comprises HCl. In some embodiments, the method comprises maintaining the coupling at a temperature of between 0 to 20° C., between −5 to 10° C., or between −10 to 10° C. during the addition of the acid. In some embodiments, the method further comprises heating the coupling to between 50 to 60° C., between 55 to 65° C., or between 60 to 70° C. In some embodiments, the method further comprises stirring the solution for between 5 to 15 hours, between 8 to 12 hours, or between 9 to 11 hours. In some embodiments, the method further comprises washing, purifying, or separating Compound S3 using a base, an aqueous solution, an alcohol, an organic solvent, or a combination thereof. In some embodiments, the base is sodium hydroxide. In some embodiments, the aqueous solution is a sodium chloride solution. In some embodiments, the sodium chloride solution is between 5 to 25% NaCl, between 10 to 30% NaCl, or between 15 to 25% NaCl. In some embodiments, the alcohol is ethanol, 2-propanol, or isopropyl alcohol. In some embodiments, the alcohol is methanol, ethanol, propanol, butanol, pentanol, hexanol, heptanol, octanol, nonanol, decanol, undecanol, or dodecanol. In some embodiments, the alcohol is a cyclic alcohol (e.g., benzyl alcohol, a sterol, an inositol, cyclohexanol, methylcyclohexanol, dimethylcyclohexanol, or a terineol). In some embodiments, the organic solvent is ethyl acetate or acetone. In some embodiments, the organic solvent is dichloromethane, dichloroethane, chloroform, carbon tetrachloride, chlorobenzene, diethyl ether, 1,4-dioxane, tert-butyl methyl ether (TBME), tetrahydrofuran (THF), N,N-dimethylformamide (DMF), N-methylpyrrolidone (NMP), diglyme, nitromethane, 1,2-dimethoxyethane (DME), pyridine, acetone, acetonitrile, benzene, o-xylene, m-xylene, p-xylene, xylenes, hexanes, cyclohexane, heptane, octane, nonane, and decane, or a combination thereof. In some embodiments, the method further comprises adding a base to the purification increasing the pH to a basic pH. In some embodiments, the base comprises about 2N to 12N of the base. In some embodiments, the base is sodium hydroxide. In some embodiments, the basic pH is between about 10 to 12.

### Compositions

The endoxifen compositions as described herein may be formulated as compositions for medical use. The compositions may have varied amounts of active pharmaceutical ingredients (APIs) (e.g., endoxifen, or any one of Compound 1-Compound 8, or combinations thereof). In some embodiments, the amount of APIs in the composition may be dependent on intended use or treatment. In some embodiments, the composition may comprise from 0.01 mg to 200 mg, from 0.1 mg to 100 mg, from 0.1 mg to 50 mg, from 0.1 mg to 20 mg, or from 1 mg to 20 mg of the API. In some embodiments, the composition may comprise from 0.01 mg to 200 mg, from 0.1 mg to 100 mg, from 0.1 mg to 50 mg, from 0.1 mg to 20 mg, or from 1 mg to 20 mg of endoxifen. In some embodiments, the composition may comprise from 0.01 mg to 200 mg, from 0.1 mg to 100 mg, from 0.1 mg to 50 mg, from 0.1 mg to 20 mg, or from 1 mg to 20 mg of the (Z)-endoxifen. In some embodiments, the composition may comprise 0.01 mg to 200 mg, from 0.1 mg to 100 mg, from 0.1 mg to 50 mg, from 0.1 mg to 20 mg, or from 1 mg to 20 mg of the endoxifen. In some embodiments, the composition may comprise 0.01 mg to 200 mg, from 0.1 mg to 100 mg, from 0.1 mg to 50 mg, from 0.1 mg to 20 mg, or from 1 mg to 20 mg of Compound 1. In some embodiments, the composition may comprise 0.01 mg to 200 mg, from 0.1 mg to 100 mg, from 0.1 mg to 50 mg, from 0.1 mg to 20 mg, or from 1 mg to 20 mg of Compound 2. In some embodiments, the composition may comprise 0.01 mg to 200 mg, from 0.1 mg to 100 mg, from 0.1 mg to 50 mg, from 0.1 mg to 20 mg, or from 1 mg to 20 mg of Compound 3. In some embodiments, the composition may comprise 0.01 mg to 200 mg, from 0.1 mg to 100 mg, from 0.1 mg to 50 mg, from 0.1 mg to 20 mg, or from 1 mg to 20 mg of Compound 4a. In some embodiments, the composition may comprise 0.01 mg to 200 mg, from 0.1 mg to 100 mg, from 0.1 mg to 50 mg, from 0.1 mg to 20 mg, or from 1 mg to 20 mg of Compound 4b. In some embodiments, the composition may comprise 0.01 mg to 200 mg, from 0.1 mg to 100 mg, from 0.1 mg to 50 mg, from 0.1 mg to 20 mg, or from 1 mg to 20 mg of Compound 5a. In some embodiments, the composition may comprise 0.01 mg to 200 mg, from 0.1 mg to 100 mg, from 0.1 mg to 50 mg, from 0.1 mg to 20 mg, or from 1 mg to 20 mg of Compound 5b. In some embodiments, the composition may comprise 0.01 mg to 200 mg, from 0.1 mg to 100 mg, from 0.1 mg to 50 mg, from 0.1 mg to 20 mg, or from 1 mg to 20 mg of Compound 6a. In some embodiments, the composition may comprise 0.01 mg to 200 mg, from 0.1 mg to 100 mg, from 0.1 mg to 50 mg, from 0.1 mg to 20 mg, or from 1 mg to 20 mg of Compound 6b. In some embodiments, the composition may comprise 0.01 mg to 200 mg, from 0.1 mg to 100 mg, from 0.1 mg to 50 mg, from 0.1 mg to 20 mg, or from 1 mg to 20 mg of Compound 7a. In some embodiments, the composition may comprise 0.01 mg to 200 mg, from 0.1 mg to 100 mg, from 0.1 mg to 50 mg, from 0.1 mg to 20 mg, or from 1 mg to 20 mg of Compound 7b. In some embodiments, the composition may comprise 0.01 mg to 200 mg, from 0.1 mg to 100 mg, from 0.1 mg to 50 mg, from 0.1 mg to 20 mg, or from 1 mg to 20 mg of Compound 8.

### Formulations

In some embodiments, the compositions or pharmaceutical compositions described herein are formulated as a suspension, a tablet, a capsule, a caplet, a liquid, or a gel. In some embodiments, a pharmaceutical composition of the present disclosure is formulated for oral delivery. Compositions intended for oral use may be prepared in solid or fluid unit dosage forms. In at least some embodiments, the compositions are formulated for oral delivery as tablets, caplets, capsules, pills, powders, troches, elixirs, suspensions, syrups, wafers, chewing gums, dragees, lozenges, and the like. In some embodiments a suspension may comprise an alcohol, a plant oil, a mineral oil, a glycol, an agar, or a mixture thereof. In some embodiments the alcohol may comprise ethanol. In some embodiments, the formulation may be formulated as an enteric formulation.

In some embodiments, the oral dosage forms are solid oral dosage forms such as tablets, caplets, and capsules. In some embodiments, the capsule is a hard capsule or a soft capsule. In other embodiments, the capsule is a gelatin capsule, gelatin-free capsule, a “cap-in-cap” capsule, alginate capsule, hydroxypropyl methyl cellulose (HPMC) capsule, a polyvinyl alcohol (PVA) capsule, a hypromellose capsule, or a starch capsule. In some embodiments, a composition may comprise hydroxypropyl methyl cellulose. In some embodiments, the composition may be formulated as a delayed release composition.

### Excipients

In some embodiments, an oral composition comprising an endoxifen composition further comprises one or more excipients. In some embodiments, an oral composition comprising endoxifen or a composition thereof further comprises one or more excipients. Accordingly, compositions designed for oral administration can be made with an inert or active excipient or with an edible carrier as disclosed herein.

In various embodiments, the composition provided herein comprises from about 1% to about 99.99%, about 5% to about 95%, about 5% to about 90%, about 10% to about 80%, about 15% to about 70%, about 20% to about 60%, from about 30% to about 95%, from about 50% to about 90%, from about 60% to about 90%, from about 60% to about 80%, or from about 70% to about 80% by weight of one or more excipients. In certain embodiments, the composition provided herein comprises about 99.99%, about 95%, about 90%, about 85%, about 80%, about 75%, about 70%, about 65%, about 60%, about 55%, or about 50% by weight of one or more excipients. In certain embodiments, the composition provided herein comprises about 99.99%, about 99%, about 98%, about 97%, about 96%, about 95%, about 94%, about 93%, about 92%, about 91%, about 90%, about 89%, about 88%, about 87%, about 86%, or about 85% by weight of one or more excipients. In certain embodiments, the composition provided herein comprises about 85%, about 84%, about 83%, about 82%, about 80%, about 79%, about 78%, about 77%, about 76%, about 75%, about 74%, about 73%, about 72%, about 71%, about 70%, about 69%, about 68%, about 67%, about 66%, or about 65% by weight of one or more excipients. In certain embodiments, the composition provided herein comprises about 55%, about 54%, about 53%, about 52%, about 51%, about 50%, about 49%, about 48%, about 47%, about 46%, or about 45% by weight of one or more excipients. In certain embodiments, the composition provided herein comprises about 30%, about 29%, about 28%, about 27%, about 26%, about 25%, about 24%, about 23%, about 22%, about 21%, or about 20% by weight of one or more excipients.

Examples of excipients that can be used in the compositions formulated for oral administration are provided herein and can include, but are not limited to, one or more of bulking agents, binders, fillers, disintegrating agents, lubricants, glidants, control release agents, enteric coatings, film-forming agents, plasticizers, colorants, sweeteners, flavoring agents and the like, or any combination thereof.

Binders suitable for use in the pharmaceutical compositions provided herein include, but are not limited to, sucrose, starches such as corn starch, potato starch, or starches such as starch paste, pregelatinized starch, and starch 1500, PEG 6000, methocel, walocel HM, Luvitec, Luvicaparolactam, Avicel, SMCC, UNIPURE, gelatin, natural and synthetic gums such as acacia, sodium alginate, alginic acid, other alginates, tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, polyvinyl pyrrolidone, hydroxypropyl methyl cellulose, (e.g., Nos. 2208, 2906, 2910), microcrystalline cellulose, and mixtures thereof. Suitable forms of microcrystalline cellulose include, but are not limited to, the materials sold as AVICEL PH 101, AVICEL PH 103 AVICEL RC 581, AVICEL PH 105 (available from FMC Corporation, American Viscose Division, Avicel Sales, Marcus Hook, Pa.), and mixtures thereof. In some embodiments, the binder is a mixture of microcrystalline cellulose and sodium carboxymethyl cellulose. Suitable anhydrous or low moisture excipients or additives include AVICEL PH 103 and Starch 1500 LM.

Examples of fillers suitable for use in the pharmaceutical compositions provided herein include, but are not limited to, talc, calcium carbonate (e.g., granules or powder), sugars such as dextrose, sucrose, lactose, a salt such as calcium carbonate, calcium phosphate, sodium carbonate, sodium phosphate, starches, microcrystalline cellulose, powdered cellulose, cellulosic bases such as methyl cellulose, carboxymethyl cellulose dextrates, kaolin, mannitol, silicic acid, sorbitol, starch, pregelatinized starch, and mixtures thereof.

One or more binder or filler in compositions is typically present in from about 10% to about 99% (wt/wt) of the composition or the dosage form. In some embodiments, binders and/or fillers in a composition comprise about 15% to 99%, about 20% to 60%, about 25% to 55%, about 30% to 50%, about 35% to 60%, about 50% to 99% (wt/wt) of the composition.

Disintegrants can be used in the compositions to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms. In some embodiments, the disintegrant is deep in the oral solid dosage form to delay disintegration. The amount of disintegrant used varies based upon the type of formulation, and is readily discernible to those of ordinary skill in the art.

Typical compositions comprise from 0.5% to 15% (wt/wt) of disintegrant. In some embodiments, compositions comprise from 1% to 5% (wt/wt) of disintegrant in the composition. In another embodiment, the disintegrant is 1% to 25%, 2% to 20%, 5% to 15%, 8% to 12%, or about 10% (wt/wt) of the composition.

Disintegrants that can be used in the pharmaceutical compositions provided herein include, but are not limited to, agar, alginic acid, calcium carbonate, microcrystalline cellulose, croscarmellose sodium, crospovidone, polacrilin potassium, sodium starch glycolate, potato or tapioca starch, pre-gelatinized starch, other starches, clays, other algins, other celluloses, gums, and mixtures thereof.

Lubricants that can be used in the pharmaceutical compositions provided herein include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, magnesium stearate or potassium stearate, ethyl oleate, ethyl laureate, agar, and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB O SIL (a pyrogenic silicon dioxide product sold by Cabot Co. of Boston, Mass.), Q7-9120 (Dow Corning), and mixtures thereof. If used at all, lubricants are typically used in an amount of less than 1% (wt/wt) of the compositions or dosage forms into which they are incorporated. In yet another embodiment, the lubricant is 0.1% to 3%, such as 0.5% to 1% (wt/wt), of the composition.

Plasticizers may be added to control the softness or pliability of oral dosage forms such as shell of a capsule, caplet or a tablet and thus, may improve the mechanical properties of the pH-sensitive materials of the coatings on the oral dosage forms. Suitable plasticizers, include, without limitation, petroleum oils (for e.g., a paraffinic process oil, a naphthenic process oil, and an aromatic process oil), squalene, squalane, plant oils, (e.g., olive oil, camelia oil, castor oil, tall oil, and a peanut oil), silicon oils, dibasic acid esters, (e.g., dibutyl phthalate, and dioctyl phthalate), liquid rubbers (e.g., polybutene and a liquid isoprene rubber), liquid fatty acid esters (e.g., isopropyl myristate ISM), hexyl laurate, diethyl sebacate, and diisopropyl sebacate, triethyl citrate, triacetin, diethylene glycol, polyethylene glycols, polypropylene glycol, phthalates, sorbitol, glycol salicylate, crotaminton, and glycerin or mixtures thereof. The amount of plasticizer may vary depending upon the chemical composition of the pharmaceutical preparation. In one embodiment, the at least one plasticizer is sorbitol, dimethyl isosorbide, or a glycerol. In another embodiment, the plasticizer is 1% to 10%, such as 3% to 5% (wt/wt), of the composition.

Examples of glidants include, but are not limited to, colloidal silicone dioxide, cellulose, calcium phosphate, di or tri-basic and the like.

As an example of sweeteners or sweetening agents include sucrose, saccharin, dextrose, maltose, sugar substitutes, aspartame, xylitol, mannitol, cyclamate, sucralose, maltitol, sorbitol, acesulfame K and the like.

Examples of flavoring agents include peppermint, methyl salicylate, peppermint, spearmint, methyl salicylate, raspberry, red berry, strawberry, pineapple, orange, cherry and the like.

Compositions formulated for oral delivery as disclosed herein, for example, tablets, caplets, and capsules, may be coated with one or more enteric coating agent, control release agent or film forming agent to control or delay disintegration and absorption of the compositions comprising endoxifen or salts thereof in the gastrointestinal tract and thereby provide a sustained action over a longer period of time. Accordingly, in some embodiments, the tablet can be an enteric tablet, the caplet can be an enteric caplet, or the capsule can be an enteric capsule. The enteric tablets, enteric caplets, or enteric capsules of the present disclosure may be prepared by techniques known in the art.

Pharmaceutical preparations disclosed herein may comprise a control release agent. Examples of control release agent suitable for use include, without limitation, pH-dependent polymers, acid-insoluble polymers, methyl acrylate-methacrylic acid copolymers, cellulose acetate phthalate (CAP), cellulose acetate succinate, hydroxypropyl methyl cellulose phthalate, hydroxypropyl methyl cellulose acetate succinate (hypromellose acetate succinate), polyvinyl acetate phthalate (PVAP), methyl methacrylate-methacrylic acid copolymers, shellac, cellulose acetate trimellitate, sodium alginate, zein, waxes, including synthetic waxes, microcrystalline waxes, paraffin wax, carnauba wax, and beeswax; polyethoxylated castor oil derivatives, hydrogenated oils, glyceryl mono-, di-tribenates, glyceryl monostearate, glyceryl distearate, long chain alcohols, such as stearyl alcohol, cetyl alcohol, and polyethylene glycol; and mixtures thereof. In some embodiments, a time delay material such as glyceryl monostearate or glyceryl distearate may be used. In other embodiments, the controlled release reagent is a digestible waxy substance such as hard paraffin wax.

In some embodiments, compositions may comprise one or more of pH-dependent polymers such as acid insoluble polymers. The pH-dependent polymers become increasingly permeable above pH 5.0 but are impermeable at pH below 5.0 whereas acid insoluble polymers become soluble in neutral to weakly alkaline conditions. Such control release polymers target upper small intestines and colon. Non-limiting examples of acid-insoluble polymers include cellulose acetate phthalate, cellulose acetate butyrate, hydroxypropyl methyl cellulose phthalate, alginic acid salts such as sodium or potassium alginate, shellac, pectin, acrylic acid-methylacrylic acid copolymers (commercially available under the tradename EUDRAGIT® L and EUDRAGIT® S from Rohm America Inc., Piscataway, NJ as a powder or a 30% aqueous dispersion; or under the tradename EASTACRYL®, from Eastman Chemical Co., Kingsport, TN, as a 30% dispersion). Additional examples include EUDRAGIT® L100-55, EUDRAGIT® L30D-55, EUDRAGIT® L100, EUDRAGIT® L100 12,5, EUDRAGIT® S100, EUDRAGIT® 512,5, EUDRAGIT® FS 30D, EUDRAGIT® E100, EUDRAGIT® E 12,5, and EUDRAGIT® PO. In at least one embodiment, the composition comprises EUDRAGIT® L100-55. EUDRAGIT® RS and RL and EUDRAGIT® NE and NM are also useful polymers for the purpose of this disclosure. In some embodiments, the composition comprises EUDRAGIT® L30D 55. In another embodiment, the preparation comprises EUDRAGIT® FS 30D. One of skill in the art will recognize that at least some acid insoluble polymers listed herein will also be biodegradable.

In some embodiments, compositions may comprise an enteric material for formulation of time delay or delayed-release formulations. For time delay or delayed-release pharmaceutical preparations of oral dosage forms, glyceryl monostearate, glyceryl distearate, and acid-insoluble polymers, for example polymethacrylate pH-sensitive polymer-based coatings can be used, (e.g., as coating material, i.e., enteric coating agents, for enteric coating of capsules, caplets, and tablets). Commercial sources for delayed-release oral dosage forms are available, for example DRCaps made of hypromellose (HPMC) from Capsugel, USA. Such delayed-release oral dosage forms are acid-resistant and can resist acidity as seen in stomach for at least 30 min, such as for at least 1 hour, for at least 1.5 hour, or for at least 2 hours. Such delayed release oral dosage forms can release at least 40%, at least 50%, at least 60%, at least 70%, at least 80% or at least 90% of the endoxifen or compositions thereof in the intestines (small intestines, large intestine/colon etc.).

In an aspect of the present disclosure, the enteric tablets, enteric caplets, and enteric capsules may be uncoated. Hard uncoated capsules with enteric capability using intrinsically enteric capsule technology (for example, EnTrinsic Drug Delivery available from Capsugel) are suitable for the purpose of the present disclosure.

In various embodiments, the enteric tablet is a hard tablet made with free-flowing powder of endoxifen or a composition thereof. In various embodiments, the enteric capsule is a capsule made with free-flowing powder of (Z)-endoxifen or a composition thereof. In various embodiments, the enteric tablet is a hard tablet made with free-flowing powder of endoxifen or a composition thereof. In various embodiments, the enteric capsule is a capsule made with free-flowing powder of endoxifen or a composition thereof.

In some embodiments, the enteric capsule is a non-animal based capsule, such as a hypromellose capsule (for example, commercially available self-gelling Vcaps, VCaps Plus, VCaps enteric, other enteric capsules made using Xcellodose, ENCODE colonic delivery technology, and EnTrinsic™ drug delivery technology from Capsugel). Other technologies known in the art and available commercially (for example, Qualicaps, USA, Nutrascience, USA, etc.) for the formulating enteric forms of oral solid dosage forms can also be utilized. In at least one embodiment, the capsule is an API-in-capsule, meaning that the (Z)-endoxifen or composition thereof is filled neat into the capsule. In such API-in-capsule oral dosage forms, the active ingredient, (Z)-endoxifen or compositions thereof can be free flowing powders or micronized powders. When the dosage form is a capsule, in at least one embodiment, the capsule can be a seamless capsule or a banded capsule.

In some embodiments, the dosage form is formulated as a suspension. The suspension may comprise microparticles of endoxifen suspended in a liquid. The liquid may have high viscosity, medium viscosity, or low viscosity. The suspension may be formulated as a syrup, an elixir, a drink, a solution to be administered orally or intravenously, a gel, an ointment, or a medicament. In some embodiments, the suspension may comprise an alcohol, a plant oil, a mineral oil, a glycol, an agar, or a mixture thereof.

Rapid absorption and bioavailability of the anti-cancer therapeutics such as endoxifen that can further reduce the cancer growth rate are highly desirable. In an aspect, the present disclosure provides that the compositions are formulated for certain pharmacokinetic (PK) properties.

The formulations and compositions as described herein may be administered at a dosage proportional to a subject's weight (e.g., a mg/kg unit dose). In some embodiments the compositions are administered at unit dose between 0.1 mg/kg and 10 mg/kg. In some embodiments, the compositions may comprise a 0.05 mg/kg unit dose, a 0.1 mg/kg unit dose, a 0.5 mg/kg unit dose, a 1 mg/kg unit dose, or a 1.5 mg/kg unit dose of the endoxifen. In some embodiments, a dose may be increased over time.

The formulations and compositions disclosed herein may include low levels of solvent and impurities. As non-limiting examples, the formulations and compositions disclosed herein may include one or more characteristics listed in TABLES 13-15 of EXAMPLE 13. In particular, a composition or formulation of the present disclosure can comprise less than 2.5%, less than 2%, less than 1.5%, less than 1%, less than 0.75%, less than 0.5%, less than 0.25%, less than 0.2%, less than 0.15%, less than 0.1%, or less than 0.05% water content. Additionally, a composition or formulation of the present disclosure can comprise less than 1%, less than 0.8%, less than 0.6%, less than 0.4%, less than 0.2%, less than 0.1%, or less than 0.05% sulphated ash. As shown in TABLES 13-15 of EXAMPLE 13, a composition or formulation of the present disclosure can also comprise less than 6000 ppm methanol, less than 5000 ppm methanol, less than 4000 ppm methanol, less than 3000 ppm methanol, less than 2000 ppm methanol, less than 1000 ppm methanol, less than 500 ppm methanol, less than 250 ppm methanol, less than 200 ppm methanol, less than 150 ppm methanol, less than 100 ppm methanol, less than 75 ppm methanol, less than 50 ppm methanol, less than 25 ppm methanol, or less than 10 ppm methanol. Additionally, a composition or formulation of the present disclosure can comprise less than 9000 ppm ethanol, less than 6000 ppm ethanol, less than 5000 ppm ethanol, less than 4000 ppm ethanol, less than 3000 ppm ethanol, less than 2000 ppm ethanol, less than 1000 ppm ethanol, less than 500 ppm ethanol, less than 250 ppm ethanol, less than 200 ppm ethanol, less than 150 ppm ethanol, less than 100 ppm ethanol, less than 75 ppm ethanol, less than 50 ppm ethanol, less than 25 ppm ethanol, or less than 10 ppm ethanol. A composition or formulation of the present disclosure can also comprise less than 9000 ppm acetone, less than 6000 ppm acetone, less than 5000 ppm acetone, less than 4000 ppm acetone, less than 3000 ppm acetone, less than 2000 ppm acetone, less than 1000 ppm acetone, less than 500 ppm acetone, less than 250 ppm acetone, less than 200 ppm acetone, less than 150 ppm acetone, less than 100 ppm acetone, less than 75 ppm acetone, less than 50 ppm acetone, less than 25 ppm acetone, or less than 10 ppm acetone. Similarly, a composition or formulation of the present disclosure can comprise less than 9000 ppm isopropyl alcohol, less than 6000 ppm isopropyl alcohol, less than 5000 ppm isopropyl alcohol, less than 4000 ppm isopropyl alcohol, less than 3000 ppm isopropyl alcohol, less than 2000 ppm isopropyl alcohol, less than 1000 ppm isopropyl alcohol, less than 500 ppm isopropyl alcohol, less than 250 ppm isopropyl alcohol, less than 200 ppm isopropyl alcohol, less than 150 ppm isopropyl alcohol, less than 100 ppm isopropyl alcohol, less than 75 ppm isopropyl alcohol, less than 50 ppm isopropyl alcohol, less than 25 ppm isopropyl alcohol, or less than 10 ppm isopropyl alcohol. Furthermore, a composition or formulation of the present disclosure can comprise less than 1000 ppm acetonitrile, less than 500 ppm acetonitrile, less than 250 ppm acetonitrile, less than 200 ppm acetonitrile, less than 150 ppm acetonitrile, less than 100 ppm acetonitrile, less than 75 ppm acetonitrile, less than 50 ppm acetonitrile, less than 25 ppm acetonitrile, less than 10 ppm acetonitrile, or less than 5 ppm acetonitrile. A composition or formulation of the present disclosure can comprise less than 9000 ppm ethyl acetate, less than 6000 ppm ethyl acetate, less than 5000 ppm ethyl acetate, less than 4000 ppm ethyl acetate, less than 3000 ppm ethyl acetate, less than 2000 ppm ethyl acetate, less than 1000 ppm ethyl acetate, less than 500 ppm ethyl acetate, less than 250 ppm ethyl acetate, less than 200 ppm ethyl acetate, less than 150 ppm ethyl acetate, less than 100 ppm ethyl acetate, less than 75 ppm ethyl acetate, less than 50 ppm ethyl acetate, less than 25 ppm ethyl acetate, or less than 10 ppm ethyl acetate. Alternatively or in addition thereto, a composition or formulation of the present disclosure can comprise less than 1500 ppm tetrahydrofuran, 1000 ppm tetrahydrofuran, less than 500 ppm tetrahydrofuran, less than 250 ppm tetrahydrofuran, less than 200 ppm tetrahydrofuran, less than 150 ppm tetrahydrofuran, less than 100 ppm tetrahydrofuran, less than 75 ppm tetrahydrofuran, less than 50 ppm tetrahydrofuran, less than 25 ppm tetrahydrofuran, less than 10 ppm tetrahydrofuran, or less than 5 ppm tetrahydrofuran. Furthermore, a composition or formulation of the present disclosure can comprise less than 1500 ppm 2-methyltetrahydrofuran, 1000 ppm 2-methyltetrahydrofuran, less than 500 ppm 2-methyltetrahydrofuran, less than 250 ppm 2-methyltetrahydrofuran, less than 200 ppm 2-methyltetrahydrofuran, less than 150 ppm 2-methyltetrahydrofuran, less than 100 ppm 2-methyltetrahydrofuran, less than 75 ppm 2-methyltetrahydrofuran, less than 50 ppm 2-methyltetrahydrofuran, less than 25 ppm 2-methyltetrahydrofuran, less than 10 ppm 2-methyltetrahydrofuran, or less than 5 ppm 2-methyltetrahydrofuran. Additionally, a composition or formulation of the present disclosure can comprise less than 250 ppm benzene, less than 200 ppm benzene, less than 150 ppm benzene, less than 100 ppm benzene, less than 75 ppm benzene, less than 50 ppm benzene, less than 25 ppm benzene, less than 10 ppm benzene, less than 5 ppm benzene, less than 2.5 ppm benzene, less than 2 ppm benzene, less than 1.5 ppm benzene, less than 1 ppm benzene, or less than 0.5 ppm benzene. A composition or formulation of the present disclosure can also comprise less than 250 ppm mesityl oxide, less than 200 ppm mesityl oxide, less than 150 ppm mesityl oxide, less than 100 ppm mesityl oxide, less than 75 ppm mesityl oxide, less than 50 ppm mesityl oxide, less than 25 ppm mesityl oxide, less than 10 ppm mesityl oxide, less than 5 ppm mesityl oxide, less than 2.5 ppm mesityl oxide, less than 2 ppm mesityl oxide, less than 1.5 ppm mesityl oxide, less than 1 ppm mesityl oxide, or less than 0.5 ppm mesityl oxide. A composition or formulation of the present disclosure can comprise less than 500 ppm zinc, less than 250 ppm zinc, less than 200 ppm zinc, less than 150 ppm zinc, less than 100 ppm zinc, less than 75 ppm zinc, less than 50 ppm zinc, less than 25 ppm zinc, less than 10 ppm zinc, less than 5 ppm zinc, less than 2.5 ppm zinc, less than 2 ppm zinc, less than 1.5 ppm zinc, less than 1 ppm zinc. Additionally, a composition or formulation of the present disclosure can comprise less than 500 ppm titanium, less than 250 ppm titanium, less than 200 ppm titanium, less than 150 ppm titanium, less than 100 ppm titanium, less than 75 ppm titanium, less than 50 ppm titanium, less than 25 ppm titanium, less than 10 ppm titanium, less than 5 ppm titanium, less than 2.5 ppm titanium, less than 2 ppm benzene, less than 1.5 ppm titanium, less than 1 ppm titanium.

In one aspect, rapid achievement of maximal and steady state plasma levels of endoxifen is a particular aspect of the present disclosure. The present disclosure provides compositions that achieve a maximal plasma level of endoxifen ranging within 2 to 30 hours, within 3 to 20 hours, within 2 to 10 hours or within 4 to 8 hours after administration of the compositions. Accordingly, in some embodiments, time to maximal (peak) plasma level of endoxifen ranges from 2 to 10 hours after administration of the composition. In some embodiments, the time to maximal plasma level of endoxifen ranges from 4 to 8 hours after administration of a composition disclosed herein.

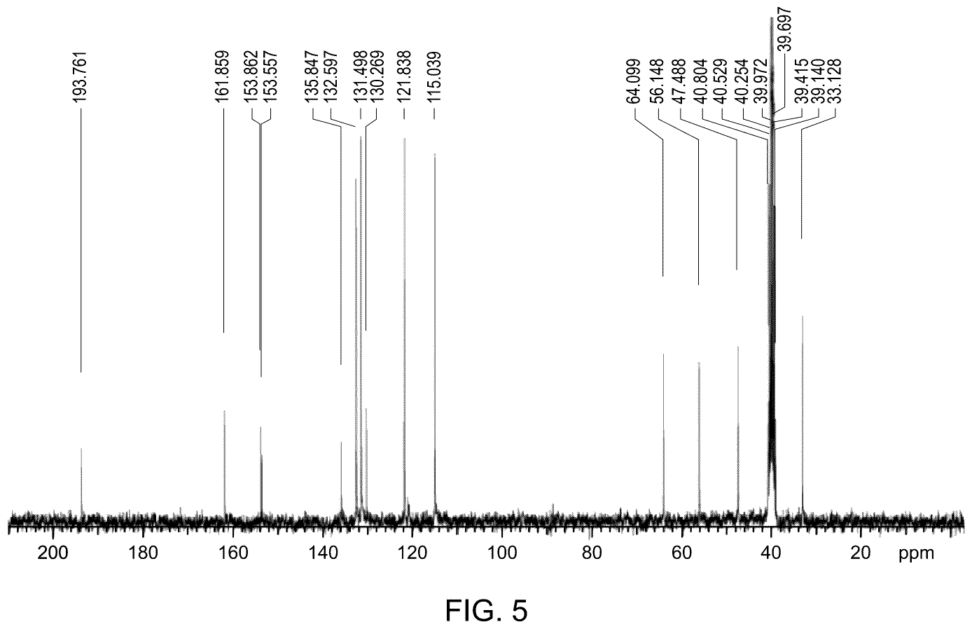

Rapid achievement of steady-state plasma levels of endoxifen is also highly desirable, and a composition of the present disclosure may provide a plasma level of endoxifen in a subject administered the composition comprising endoxifen, (Z)-endoxifen or a salt thereof, and one or more endoxifen-related compounds (e.g., any one of Compound 1-Compound 8) that rapidly achieves steady state. Steady state plasma levels can be achieved from day 7 to day 21. In some embodiments, the steady state plasma levels can be achieved by day 7 (FIG. 5) upon daily administration of a composition disclosed herein.

In an aspect, the present disclosure provides that circulating endoxifen released from a composition disclosed herein can be cleared faster than tamoxifen. Terminal elimination half-life of tamoxifen is said to be 5-7 days (Jordan C. Steroids. 2007 November; 72(13): 829-842) and peak concentration time of tamoxifen is approximately 5 hours post-dose. Endoxifen released from a composition disclosed herein can have a terminal elimination half-life ranging from 30 to 60 hours, significantly lower than tamoxifen. In some embodiments, the mean half-life ranges from 40 to 53 hours. The mean ratio of AUC₂₄ₕᵣ (Day 21)/AUC_{0-inf} (Day1) typically ranges from 0.7 to 1.2 for compositions comprising 1 mg to 4 mg (Z)-endoxifen, or a salt thereof. Thus, accumulation of endoxifen released from a composition disclosed herein does not significantly vary over continued treatment.

In another aspect, a composition described herein achieves absorption of endoxifen that is therapeutically effective.

Area under Curve AUC_{(0-24 hr)} (“AUC₂₄ₕᵣ”) describes the total exposure of the subject to a drug from time of dosing (0 hr) over a 24-hour period. Compositions comprising (Z)-endoxifen or a salt thereof typically achieve mean (AUC₂₄ₕᵣ) of 150 hr*ng/mL to 600 hr*ng/mL on Day 1 of initial (first) dose of a composition comprising 1 mg to 4 mg of (Z)-endoxifen. Compositions comprising (Z)-endoxifen or a salt thereof typically achieve mean AUC₂₄ₕᵣ of 400 hr*ng/mL to 2500 hr*ng/mL on Day 21 of initial (first) dose of compositions comprising 1 mg to 4 mg of (Z)-endoxifen.

AUC_{0-inf} (“AUC_{0-inf}”), a time-averaged concentration of drug circulating in the body fluid analyzed (normally plasma, blood or serum), describes the total exposure of the subject to a drug. The present disclosure provides that the exposure of a subject to endoxifen (AUC_{0-inf}) can be dose proportional. In some embodiments, AUC_{0-inf} ranges from 200 hr*ng/mL to 10000 hr*ng/mL. In other embodiments, the AUC_{0-inf} ranges from 300 hr*ng/mL to 8000 hr*ng/mL. In certain embodiments, the AUC_{0-inf} ranges from 400 hr*ng/mL to 6000 hr*ng/mL over the dosing range of 1 mg to 4 mg of (Z)-endoxifen.

Dissolution of the oral dosage forms disclosed herein is tested by the dissolution tests according to the current methods of USP 711. In some embodiments, the oral dosage forms disclosed herein are protected from the acidic environment of the stomach and do not dissolve for at least 2 hours, at least 3 hours, at least 4 hours, at least 5 hours, 6 hours, at least 7 hours or at least 8 hours. In at least one embodiment, the oral dosage forms do not release endoxifen for at least 6 hours. In another embodiment, the oral dosage forms do not release endoxifen or a salt thereof for at least 2 hours.

In other embodiments, less than 10% of (Z)-endoxifen in a composition comprising endoxifen or a salt thereof disclosed herein is released in the stomach after 2 hours after administration; or wherein less than 40% of (Z)-endoxifen is released in the stomach after 4 hours of administration; or less than 50% of (Z)-endoxifen is released in the stomach after 6 hours of administration, as tested by a method of USP 711.

In another embodiment, a composition disclosed herein releases in the stomach less than 10% of (Z)-endoxifen in 2 hours after administration, less than 40% of (Z)-endoxifen in 4 hours after administration; and less than 50% of (Z)-endoxifen in 6 hours after administration, as tested by a method of USP 711.

In yet another embodiment, the composition is formulated to release in the small intestine, such that at least 10% of endoxifen is released after 4 hours after administration; or at least 30% of endoxifen is released after 6 hours after administration; or at least 40% of endoxifen is released after 7 hours after administration; or at least 50% of endoxifen is released after 8 hours after administration, as tested by a method of USP 711.

In a further embodiment, as determined by a method of USP 711, the composition is formulated to release in the colon at least 50% of endoxifen after 8 hours after administration.

In still further embodiments, the composition is formulated to release in the colon at least 20% of endoxifen after 4 hours after administration; at least 40% of endoxifen after 6 hours after administration; at least 60% of endoxifen after 7 hours after administration; or at least 80% of endoxifen after 8 hours after administration.

An oral dosage form can be of any shape suitable for oral administration, such as spherical (0.05-5 mL), oval (0.05-7 mL), ellipsoidal, pear (0.3-5 mL), cylindrical, cubic, regular and/or irregular shaped. An oral dosage form may be of any size suitable for oral administration, for example, size 0, size 2, and the like.

One of skill in the art will further recognize that compositions disclosed herein may comprise one or more of the excipients known in the art and disclosed herein in any combination appropriate for a desired formulation or preparation. Additional excipients may generally be found in Remington's The Science and Practice of Pharmacy, Meade Publishing Co., United States Pharmacopeia/National Formulary. One of skill in the art will be able to select suitable excipients necessary for the preparation of the formulations and appropriate dosage forms compatible with the route of administration based on his or her skill and knowledge in the art and the disclosures made herein. In all cases, the ultimate dosage form should be sterile and stable under the conditions of manufacture and storage.

For formulations of the solid dosage compositions disclosed herein, as the water activity (A_{w}) is less than 0.75, testing Total Aerobic Plate Count (TAC) and USP indicator organism is typically not necessary. The publication, “Microbial Bioburden on Oral Solid Dosage Form,” by Jose E. Martinez, Pharmaceutical Technology, February 2002, pages 58 to 70, is hereby incorporated by reference in its entirety.

Furthermore, since formulations of the compositions disclosed herein also have water activity of less than 0.75, then detailed microbial testing is typically not necessary. TAC is an estimation of the total viable aerobic microbes present in a sample of raw material, in-process material, or finished product. Samples are analyzed in accordance with the most current USP 39 <61>, “Microbiological Examination of Nonsterile Products: Microbial Enumeration Tests.”

‘Acceptable TAC for oral solid dosage forms (OSDFs) are established for the formulation of the inventive compositions in terms of alert and action levels, which could be 1000 cfu g/mL, and 10,000 cfu g/mL, respectively. A TAC that is 20,000 cfu g/mL may be considered unacceptable.

For other formulations, such as liquid or fluid formulations with water activity of less than 0.75, Tests for Specified Microorganisms (S. aureus, Ps. aeruginosa, Salmonella, C. albicans, Clostridia, E. coli and Bile Tolerant Gram negative bacteria) in compliance with USP Guidelines Chapter 62 may not need to be performed.

### Methods of Use

Compositions comprising endoxifen and endoxifen-related compounds (e.g., any one of Compound 1-Compound 8) disclosed herein may be used in the manufacture of medicaments for use in the treatment of a subject in need thereof, for example, subjects having or at risk of having a hormone-dependent breast disorder, a hormone-dependent reproductive tract disorder, or both. In various instances, the compound used for such treatments is (Z)-endoxifen.

The compositions of the present disclosure may be used as a primary therapy, as a part of a neo-adjuvant therapy (to primary therapy), or as part of adjuvant therapy regimen, where the intention is to ameliorate or cure a subject having or at risk of having a hormone-dependent breast disorder, hormone-dependent reproductive tract disorder, or both.

In certain embodiments, the disorder is a hormone-dependent breast disorder. In other embodiments, the disorder is hormone-dependent reproductive tract disorder. In still other embodiments, the subject has both a hormone-dependent breast disorder and a hormone-dependent reproductive tract disorder. In some embodiments, the hormone dependent disorder is a benign breast disorder, hyperplasia, atypia, atypical ductal hyperplasia, atypical lobular hyperplasia, increased breast density, gynecomastia, DCIS, LCIS, breast cancer, endometrial cancer, ovarian cancer, uterine cancer, cervical cancer, vaginal cancer or vulvar cancer.

In some embodiments, the breast disorder is increased breast density. For example, the breast disorder is a class B (formerly Class II), class C (formerly class III) or class D (formerly class IV) breast density.

In some embodiments, the hormone-dependent breast disorder or hormone-dependent reproductive tract disorder is precocious puberty. In other embodiments, the hormone-dependent breast disorder or hormone-dependent reproductive tract disorder is McCune-Albright Syndrome.

In some embodiments, the breast disorder is gynecomastia. In some embodiments, gynecomastia is presented secondarily to an underlying disease. Accordingly, in some embodiments, the subject also has underlying disease selected from the group consisting of prostate cancer, cirrhosis and liver disease, male hypogonadism, hyperthyroidism, renal failure and in patients undergoing hemodialysis, or type I diabetes mellitus. In certain embodiments, the subject has prostate cancer as the underlying disease, wherein the subject has or is at risk of having gynecomastia.

In certain embodiments, the breast cancer is DCIS, LCIS, ILC, IDC, MIC, inflammatory breast cancer, ER-positive (ER+) breast cancer, HER2+ breast cancer, adenoid cystic (adenocystic) carcinoma, low-grade adenosquamous carcinoma, medullary carcinoma, mucinous (or colloid) carcinoma, papillary carcinoma, tubular carcinoma, metaplastic carcinoma, or micropapillary carcinoma. In at least one embodiment, a single breast cancer tumor may be a combination of the foregoing or be a mixture of invasive and in situ cancer.

The present disclosure contemplates the use of the compounds and compositions disclosed herein at various stages in tumor development and progression, including the treatment of advanced and/or aggressive neoplasms, i.e., overt disease in a subject that is not amenable to cure by local modalities of treatment such as surgery or radiotherapy, metastatic disease, or locally advanced disease. Accordingly, in some embodiments, the breast cancer is a pre-cancer, an early-stage cancer, a non-metastatic cancer, a pre-metastatic cancer, or a locally advanced cancer. In at least one embodiment, the breast disorder is metastatic cancer. In some embodiments, the subject further has prostate cancer.

Current choice for therapeutics for such disorders remains tamoxifen, despite serious adverse effects, poor patient compliance and resistance to the drug due to low plasma endoxifen levels seen subjects. Such subjects may have low endoxifen levels upon dosing with tamoxifen for any number of reasons, such as having CYP gene mutations, for example, in CYP2D6, CYP3A4, or CYP2C9, making them unable to metabolize tamoxifen to its active metabolite, endoxifen, or low or dysfunctional estrogen receptor preventing (or decreasing) sufficient tamoxifen uptake, for other reasons yet to be identified. Reported therapeutic levels of plasma tamoxifen in subjects dosed with 20 mg of oral tamoxifen is ≥30 nM ((Lyon et al. Genet Med. 2012 December; 14(12):990-1000). Notwithstanding the mechanism underlying the low plasma endoxifen in a subject, the compositions of the present disclosure are useful for any condition wherein a subject has low endoxifen or the subject has or is at a risk of having hormone-dependent breast disorder or hormone-dependent reproductive tract disorder. Therefore, the compositions of the present disclosure can be particularly important in the treatment of tamoxifen-resistant, hormone-dependent breast disorders or hormone-dependent reproductive tract disorders.

Provided herein in certain embodiments are patient populations for whom the pharmaceutical compositions are particularly useful. The compositions of the present disclosure are also particularly important in the treatment of tamoxifen-refractory subjects with hormone-dependent breast disorders or hormone-dependent reproductive tract disorders. Accordingly, in some embodiments, the compositions disclosed herein are useful for the treatment of tamoxifen refractory or tamoxifen resistant subjects having or at risk of having hormone-dependent breast disorders, hormone-dependent reproductive tract disorders, or both. In some embodiments, compositions comprising endoxifen and endoxifen-related compounds (e.g., any one of Compound 1-Compound 8), administered to such subject at the doses disclosed herein, will be advantageous.

Drug interactions between tamoxifen and selective serotonin reuptake inhibitors (SSRI) drugs like Prozac and Paxil (paroxetine) may occur and may be detrimental to breast cancer subjects. The SSRI drugs reduce or stop liver metabolism of tamoxifen to endoxifen in subjects on SSRI drugs. Thus, provided herein in certain embodiments are patient populations being treated or to be treated with SSRI drugs that would be benefitted by treatment with compositions of the present disclosure.

Compositions disclosed herein administered orally maintain the subject's plasma endoxifen at steady state levels greater than 30 nM, for example, at levels ranging from 30 nM to 80 nM or at levels ranging from 30 nM to 300 nM. In some embodiments, the plasma steady state endoxifen levels are maintained at >40 nM. Maintenance of such a plasma endoxifen at steady state levels greater than 30 nM is advantageous in that the likelihood of recurrence (relapse) of hormone-dependent breast disorders or hormone-dependent reproductive tract disorders, particularly breast cancer, at plasma endoxifen levels lower than 30 nM is reduced. It is particularly advantageous for subjects that are poor metabolizers of tamoxifen (with plasma endoxifen levels lower than 16 nM), intermediate metabolizers of tamoxifen (with plasma endoxifen levels lower than 27 nM) to be dosed with a composition disclosed herein. It also advantageous for subjects being treated or to be treated with antidepressant drugs such as SSRI drugs such as citalopram (Celexa), escitalopram (Lexapro), fluoxetine (Prozac), paroxetine (Paxil, Pexeva), sertraline (Zoloft), vilazodone (Viibryd) and the like, for example, a subject having or likely to have depression.

Whether a subject is tamoxifen-refractory may be determined by dosing a subject with an initial dosage of tamoxifen and determining the subject's plasma endoxifen steady state level. Plasma endoxifen steady state levels in a subject dosed with tamoxifen serves as a biomarker for the tamoxifen-refractory subjects. The plasma endoxifen levels (acute and/or steady state) may be determined by obtaining from the subject a test sample, which may be blood sample, collected from the subject after dosing the subject with tamoxifen. Plasma or serum may be obtained from blood samples for testing the biomarker endoxifen levels. The initial dosage may comprise administering tamoxifen daily for at least 1 day, 2 days, 3 days, 15 days, 1 week, 2 weeks, 4 weeks, 1 month, 2 months, 3 months, 4 months, 5 months, or 6 months. The subject may also be administered with a first composition comprising tamoxifen daily for at least 1 day, 2 days, 3 days, 15 days, 1 week, 2 weeks, 4 weeks, 1 month, 2 months, 3 months, 4 months, 5 months, 6 months, 1 year, 2 years, 3 years, 4 years, 5 years, or 10 years.

A subject's plasma endoxifen steady state level may be determined by measuring endoxifen in a test sample. The subject's plasma endoxifen steady state levels are compared to a reference plasma endoxifen level. For the purposes of the present disclosure, the reference plasma level is 30 nM. If the subject's plasma endoxifen level is determined to be lower than 30 nM, then the subject is defined as tamoxifen-refractory. Such a tamoxifen-refractory subject who has or who may be at risk of having a hormone-dependent breast disorder or hormone-dependent reproductive tract disorder is treated by administering to the subject an oral composition comprising endoxifen and endoxifen-related compounds (e.g., any one of Compound 1-Compound 8). In some embodiments, the composition administered to such a subject comprises (Z)-endoxifen free base. In other embodiments, the composition administered to such a subject comprises endoxifen gluconate selected from the group consisting of (Z)-endoxifen D-gluconate, (Z)-endoxifen L-gluconate, (E)-endoxifen D-gluconate, (E)-endoxifen L-gluconate, or a combination thereof. In other embodiments, the composition comprising endoxifen is endoxifen HCl or endoxifen citrate. The present disclosure also contemplates that a subject's plasma endoxifen levels are tracked or monitored periodically or as necessary. If required, a subject who has been administered an initial dosage of tamoxifen may have his or her plasma endoxifen steady state levels adjusted by administering a composition comprising endoxifen on an ongoing basis based on the test results.

In some embodiments, the subject's tamoxifen-refractory status may be determined by determining the subject's tamoxifen-metabolites profile which is compared with a reference tamoxifen-metabolite profile as seen in control or normal subjects. Subjects with low plasma endoxifen levels in subject's tamoxifen-metabolite profile as compared to the reference tamoxifen-metabolite profile are administered an oral composition comprising endoxifen or a salt thereof. Such compositions may comprise synthetically prepared endoxifen.

The plasma endoxifen may be measured by any of method known in the art. The levels of plasma endoxifen in test sample may be determined based on subject's genes, DNA, RNA, protein, tamoxifen-metabolite profile or a combination thereof. The tamoxifen-metabolites profile can include at least tamoxifen, 4-OHT, N-desmethyltamoxifen, and/or endoxifen. In some embodiments, the level of plasma endoxifen and/or tamoxifen-metabolite profile in the test sample is measured by High Performance Liquid Chromatography (HPLC), Gas Chromatography Mass Spectrometry (GC-MS), Liquid Chromatography Mass spectrometry (LC-MS), Liquid Chromatography Tandem Mass spectrometry (LC-MS/MS), immunohistochemistry (IHC), polymerase chain reaction (PCR), quantitative PCR (qPCR), and the like. In some embodiments, the tamoxifen-metabolites profile is predicted based on the subject's genetic composition. In some embodiments, the subject's CYP genotype includes, without limitation, analysis of CYP2D6, CYP3A4, CYP2C9 genes. In some embodiments, subject's estrogen receptor levels may be analyzed. In other embodiments, the determination of plasma endoxifen may be done by a third-party laboratory.

Accordingly, provided herein are methods of maintaining in a subject in need thereof a plasma endoxifen a level greater than 30 nM by administering to the subject a composition comprising endoxifen and endoxifen-related compounds (e.g., any one of Compound 1-Compound 8). In some embodiments, the subject's plasma endoxifen level is maintained at a steady state level greater than 30 nM. In some embodiments, the subject's plasma endoxifen levels are maintained at a steady state level ranging from 30 nM to 300 nM (for example, from 30 nM to 200 nM, or from 30 nm to 80 nM). In some embodiments, the subject's plasma endoxifen levels are maintained at a steady state level >40 nM.

In another aspect, the subjects may have their test samples tested for their biomarker profile that may be indicative or monitoring a hormone-dependent breast disorder, a hormone-dependent reproductive tract disorder, or both. Such biomarkers are known in the art and include, by way of non-limiting examples, biomarkers such as CYP2D6, BRCA-1, BRCA-2, ER, PR, Her2, uPA, PAI, Tf, p53, Ki67, cytokeratins, cancer tumor antigens, and other biomarkers measured by Mammaprint, OncotypeDx, PAM50, EndoxPredict, MammoStrat, and other diagnostic and predictive tests. A subject with biomarker profile indicating that the subject has or is at risk of having a hormone-dependent breast disorder, a hormone-dependent reproductive tract disorder, or both can be administered a composition disclosed herein. In one aspect, the present disclosure provides a method of treating a subject having or at risk of having a hormone-dependent breast disorder, a hormone-dependent reproductive tract disorder, or both, comprising determining a subject's tamoxifen-refractory or tamoxifen-resistant status and administering to the subject a composition described herein.

In some aspects, provided herein are methods of treating a tamoxifen-refractory or tamoxifen-resistant subject, the method comprising administration to the subject a composition comprising endoxifen and endoxifen-related compounds (e.g., any one of Compound 1-Compound 8).

A method of treatment may comprise daily administration of an endoxifen composition to a subject. In some embodiments, from about 10 mg to about 100 mg, from about 20 mg to about 100 mg, from about 30 mg to about 100 mg, from about 40 mg to about 100 mg, from about 60 mg to about 100 mg, from about 80 mg to about 100 mg, from about 10 mg to about 90 mg, from about 20 mg to about 90 mg, from about 30 mg to about 90 mg, from about 40 mg to about 90 mg, from about 60 mg to about 90 mg, from about 80 mg to about 90 mg, from about 10 mg to about 80 mg, from about 20 mg to about 80 mg, from about 30 mg to about 80 mg, from about 40 mg to about 80 mg, from about 50 mg to about 80 mg, from about 10 mg to about 60 mg, from about 20 mg to about 60 mg, from about 30 mg to about 60 mg, from about 40 mg to about 60 mg, from about 50 mg to about 60 mg of (Z)-endoxifen are administered to a subject per day. In some embodiments, the (Z)-endoxifen may be administered in the form of an enteric-resistant delayed release capsule comprising about 10 mg, about 20 mg, about 40 mg, or about 80 mg (Z)-endoxifen per capsule.

The compositions of the present disclosure may be administered to a subject for a duration of from about 7 to about one year. In some embodiments, the composition may be administered daily. In some embodiments, the endoxifen compositions of the present disclosure are administered until the disease or condition is treated. For example, an endoxifen composition may be administered to a subject daily for up to about 6 months or until the disease or condition is treated. In another example, an endoxifen composition may be administered to a subject daily for about 28 days to about 6 months or until the disease or condition is treated. Treating the disease or condition may comprise alleviating one or more symptoms of the disease or reducing or eliminating the disease. For example, an endoxifen composition may be administered daily to a subject having a tumor until a tumor in the subject begins to shrink, reduces in size by a predetermined amount, or is no longer detectable.

Following administration, a blood plasma concentration of endoxifen may be measured in the subject. In some embodiments, following oral administration of an endoxifen composition of the present disclosure, the blood plasma concentration of endoxifen may comprise at least about 50%, at least about 55%, at least about 60%, at least about 65%, at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% of endoxifen in the (Z)-isoform, by weight, relative to total endoxifen present in the blood plasma of the subject.

In some embodiments, an endoxifen composition may be administered to a subject in combination with an additional therapeutic agent. Examples of additional therapeutic agents that may be used in combination with the endoxifen formulations of the present disclosure include but are not limited to bicalutamide, enzalutamide, or an anticancer drug such as trastuzumab, antineoplastics such as capecitabine (e.g., XELODA®), carboplatin (e.g., PARAPLATIN®), cisplatin (e.g., PLATINOL®), cyclophosphamide (e.g., NEOSAR®), docetaxel (e.g., DOCEFREZ®, TAXOTERE®), doxorubicin (e.g., ADRIAMYCIN®), PEGylated liposomal doxorubicin (e.g., DOXIL®), epirubicin (e.g., ELLENCE®), fluorouracil (5-FU, e.g., ADRUCIL®), gemcitabine (e.g., GEMZAR®), methotrexate (multiple brand names), paclitaxel (e.g., TAXOL®), protein-bound paclitaxel (e.g., ABRAXANE®), vinorelbine (e.g., NAVELBINE®), eribulin (e.g., HALAVEN®), ixabepilone (e.g., IXEMPRA®), and ATP-cassette binding protein transport inhibitors.

As used herein, the singular forms “a,” “an” and “the” include plural referents unless the context clearly dictates otherwise.

As used herein, the terms “about” and “approximately,” in reference to a number, is used herein to include numbers that fall within a range of 10%, 5%, or 1% in either direction (greater than or less than) the number unless otherwise stated or otherwise evident from the context (except where such number would exceed 100% of a possible value).

A composition can be administered alone or in combination with other treatments, either simultaneously or sequentially dependent upon the condition to be treated.

As used herein, the term “subject” broadly refers to any animal, including but not limited to, human and non-human animals (e.g., dogs, cats, cows, horses, sheep, pigs, poultry, fish, crustaceans, etc.).

As used herein, the term “effective amount” refers to the amount of a composition sufficient to effect beneficial or desired results. An effective amount can be administered in one or more administrations, applications or dosages and is not intended to be limited to a particular formulation or administration route.

As used herein, the term “therapeutically effective amount” is an amount that is effective to ameliorate a symptom of a disease. A therapeutically effective amount can be a “prophylactically effective amount” as prophylaxis can be considered therapy.

As used herein, the terms “administration” and “administering” refer to the act of giving a drug, prodrug, or other agent, or therapeutic treatment to a subject or in vivo, in vitro, or ex vivo cells, tissues, and organs. Exemplary routes of administration to the human body can be through space under the arachnoid membrane of the brain or spinal cord (intrathecal), the eyes (ophthalmic), mouth (oral), skin (topical or transdermal), nose (nasal), lungs (inhalant), oral mucosa (buccal or lingual), ear, rectal, vaginal, by injection (e.g., intravenously, subcutaneously, intratumorally, intraperitoneally, etc.) and the like.

As used herein, the term “treatment” means an approach to obtaining a beneficial or intended clinical result. The beneficial or intended clinical result can include alleviation of symptoms, a reduction in the severity of the disease, inhibiting an underlying cause of a disease or condition, steadying diseases in a non-advanced state, delaying the progress of a disease, and/or improvement or alleviation of disease conditions.

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), glycerol, liquid polyethylene glycols, aprotic solvents such as dimethylsulfoxide, N-methylpyrrolidone and mixtures thereof, and various types of wetting agents, solubilizing agents, anti-oxidants, bulking agents, protein carriers such as albumins, any and all solvents, dispersion media, coatings, sodium lauryl sulfate, isotonic and absorption delaying agents, disintegrants (e.g., potato starch or sodium starch glycolate), and the like. The compositions also can include stabilizers and preservatives. For examples of carriers, stabilizers and adjuvants, see, e.g., Martin, Remington's Pharmaceutical Sciences, 21th Ed., MackPubl. Co., Easton, Pa. (2005), incorporated herein by reference in its entirety.

The term “inhibit” or “inhibition of” means to reduce by a measurable amount, or to prevent entirely (e.g., 100% inhibition).

Compounds are described using standard nomenclature. Unless otherwise defined, all technical and scientific terms used herein have the same meaning as is commonly understood by one of skill in the art to which this invention belongs.

As used herein, the terms “active pharmaceutical ingredient”, “active ingredient”, “API,” “drug,” “active,” “actives” or “therapeutic agent” may be used interchangeably to refer to the pharmaceutically active compound(s) in a pharmaceutical composition. This is in contrast to other ingredients in the compositions, such as excipients, which are substantially or completely pharmaceutically inert. A suitable API in accordance with the present disclosure is one where there is or likely may be patient compliance issues for treating a certain disease, condition, or disorder. The therapeutic agent as used herein includes the active compound and its salts, prodrugs, and metabolites. As used herein the term “drug” means a compound intended for use in diagnosis, cure, mitigation, treatment, and/or prevention of disease in man or other animals.

As used herein, “adjuvant therapy” refers to a therapy that follows a primary therapy and that is administered to subjects at risk of relapsing. Adjuvant systemic therapy in case of breast cancer or reproductive tract cancer, for example with tamoxifen, usually begins soon after primary therapy to delay recurrence, prolong survival or cure a subject.

As used herein, the term “tamoxifen” refers to (Z)-2-[4-(1,2-diphenyl-1-butenyl)phenoxy]-N,N-dimethylethanamine. Tamoxifen can also refer to the E-isomer or a combination of the E-isomer and the Z-isomer.

As used herein, the terms “4-hydroxytamoxifen,” “afimoxifene,” and “4-OHT” used interchangeably refer to 4-1-[4-[2-(dimethylamino)ethoxy]phenyl]-2-phenylbut-1-enyl]phenol, and constitutes an active metabolite of tamoxifen. 4-OHT can refer to the Z-isomer, E-isomer or a combination thereof.

As used herein, the term “endoxifen” refers to 4-hydroxy-N-desmethyl-tamoxifen. It is a secondary active metabolite of tamoxifen.

All compounds disclosed herein are further understood to include all possible isotopes of atoms occurring in the compounds. Isotopes include those atoms having the same atomic number but different mass numbers. By way of example, and without limitation, isotopes of hydrogen include tritium and deuterium and isotopes of carbon include ¹¹C, ¹³C and ¹⁴C.

As used herein and in the claims, the terms “comprising,” “containing,” and “including” are inclusive, open-ended and do not exclude additional unrecited elements, compositional components or method steps. Accordingly, the terms “comprising” and “including” encompass the more restrictive terms “consisting of” and “consisting essentially of”

As used herein, the term “combination therapy” refers to the use of a composition described herein in combination with one or more additional treatment. Treatment in combination therapy can be any treatment such as any prophylactic agent, therapeutic agent (such as chemotherapy), radiotherapy, surgery and the like. The combination can refer to inclusion of a therapeutic or prophylactic agent in a same composition as a composition disclosed herein (for example, in the same capsule, tablet, ointment, etc.) or in separate compositions (for example, in 2 separate capsules). The separate compositions may be in a different dosage form. The use of the terms “combination therapy” and “in combination with” does not restrict the order in which a composition described herein and prophylactic and/or therapeutic agent and/or treatment are administered to a subject in need thereof. Compositions of the present disclosure can be administered prior to (e.g., 1 minute (min), 5 min, 15 min, 30 min, 45 min, 1 hour (h), 2 h, 4 h, 6 h, 8 h, 10 h, 12 h, 24 h, 36 h, 48 h, 72 h, 96 h, 1 week (wk), 2 wk, 3 wk, 4 wk, 5 wk, 6 wk, 8 wk, 12 wk, 6 months (m), 9 m, or 1 year before), concomitant with, or subsequent to (e.g., 1 minute (min), 5 min, 15 min, 30 min, 45 min, 1 hour (h), 2 h, 4 h, 6 h, 8 h, 10 h, 12 h, 24 h, 36 h, 48 h, 72 h, 96 h, 1 week (wk), 2 wk, 3 wk, 4 wk, 5 wk, 6 wk, 8 wk, 12 wk, 6 months (m), 9 m, or 1 year after) administration of one or more prophylactic and/or therapeutic agent and/or treatment to a subject in thereof. Combination therapy as used herein can also refer to treatment of a subject having a single disease or multiple diseases, for example, prostate cancer in men and gynecomastia.

As used herein, the term “test sample” means sample of blood obtained from a subject. It is to be understood that when blood sample is obtained from a subject, subject's blood is used for determining the subject's endoxifen levels and/or other biomarkers that may be measured or tested. As used herein “plasma endoxifen” is used to refer to endoxifen levels in the subject's test sample, whether the test is conducted on whole blood, plasma or serum.

As used herein, the term “dosage form” means the form in which the compounds or compositions of the present disclosure are delivered to a patient.

As used herein, the term “pharmaceutically acceptable” or “pharmacologically acceptable” means materials, compositions, or vehicles that are compatible with other ingredients of the formulation and that they do not substantially produce adverse reactions, e.g., toxic, allergic, or immunological reactions, when administered to a subject. They may be approved by a regulatory agency, e.g., of the U.S. Federal or state government or listed in the U.S. pharmacopeia or other generally recognized pharmacopeia for use in animals, and more particularly in humans.

As used herein, the term “pharmaceutically acceptable salt” refers to any salt (e.g., obtained by reaction with an acid or a base) of a compound of the present disclosure that is physiologically tolerated in a subject (e.g., a mammal, and/or in vivo, ex-vivo, in vitro cells, tissues, or organs). A “salt” of a compound of the present disclosure may be derived from inorganic or organic acids and bases. Suitable anion salts include, arecoline, besylate, bicarbonate, bitartarate, butylbromide, citrate, camysylate, gluconate, glutamate, glycollylarsanilate, hexylresorcinate, hydrabamine, hydrobromide, hydrochloride, hydroxynapthanoate, isethionate, malate, mandelate, mesylate, methylbromide, methylbromide, methylnitrate, methylsulfate, mucate, napsylate, nitrate, pamaoate (Embonate), pantothenate, phosphate/diphosphate, polygalacuronate, salicylate, stearate, sulfate, tannate, teoclate, fatty acid anions, and triethiodide.

Suitable cations include benzathine, clemizole, chloroprocaine, choline, diethylamine, diethanolamine, ethylenediamine, meglumine, piperazine, procaine, aluminum, barium, bismuth, lithium, magnesium, potassium, and zinc.

For the purposes of this application, the salts of the compounds of the present disclosure are contemplated to be pharmaceutically acceptable for therapeutic uses. However, salts of acids and bases that are non-pharmaceutically acceptable may also be useful, for example, in the preparation or purification of a pharmaceutically acceptable compound.

As used herein, the term “pharmaceutical composition” means a combination of the active agent (e.g., an active pharmaceutical compound or ingredient, API) with a carrier, inert or active (e.g., a phospholipid), making the compositions specially suitable for diagnostic or therapeutic uses in vitro, in vivo, or ex vivo.

As used herein “primary therapy” refers to a first line of treatment upon initial diagnosis of a hormone-dependent breast disorder, a hormone-dependent reproductive tract disorder, or both in a subject. Exemplary primary therapies may involve surgery, a wide range of chemotherapies, and radiotherapy.

As used herein, the term “tamoxifen refractory” refers to a subject that has been dosed daily with tamoxifen for at least 2 days and has a level of plasma endoxifen of less than 30 nM (e.g., less than 20 nM, less than 25 nM, or less than 30 nM). As used herein, the term “tamoxifen resistance” refers to two classes of resistance: (a) de novo resistance, i.e., non-responsiveness to tamoxifen therapy from the beginning of the treatment, or (b) acquired resistance, i.e., non-responsiveness to tamoxifen therapy after initial responsiveness or tamoxifen-dependent growth/stimulated growth while continuing to express estrogen receptors (Minsun Chang. Biomol. Ther. 20(3), 256-267 (2012)). The acquired resistance to tamoxifen may develop as early as 3 m to 1 year to as late as 5 to 10 years. As used herein, the term “reference plasma endoxifen level” refers to a value of 30 nM.

As used herein, the term “unit dosage form” refers to physically discrete units suitable for unitary dosages for subjects, each unit containing a predetermined quantity of active material calculated to produce the desired therapeutic effect, in association with a suitable pharmaceutical excipient.

It is specifically understood that any numerical value cited herein includes all values from the lower value to the upper value, i.e., all possible combination of numerical values between the lowest value and the highest value enumerated are to be considered to be expressly stated in this application and the endpoint of all ranges are included within the range and independently combinable. For example, if a concentration range or beneficial range is stated as 1% to 50%, it is intended that values such as 2% to 40%, 10% to 30%, or 1% to 3% etc., are expressly enumerated in this specification. It is also to be understood that if a concentration or dose is stated as a specific value such as 1 mg or 10 mg, it is intended that it is intended to include 10% variation. As another example, a stated concentration of 20% is intended to include values+10%. Yet another example, if a ratio of 1:10 to 10:1 is stated, then it is intended that ratios such as 1:9 to 9:1, from 1:8 to 8:1, from 1:7 to 7:1, from 1:6 to 6:1, from 1:5 to 5:1, from 1:4 to 4:1, from 1:3 to 3:1, from 1:2 to 2:1, from 1:1 to 2:1 or from 2:5 to 3:5 etc. are specifically intended. There are only some examples of what is specifically intended. Unless specified otherwise, the values of the constituents or components of the compositions are expressed in weight percent of each ingredient in the component.

All methods described herein can be performed in a suitable order unless otherwise indicated or otherwise clearly contradicted by context. The use of any and all examples, or exemplary language (e.g., “such as” and “the like”) is intended merely to illustrate the invention and does not pose a limitation on the scope of the invention unless otherwise claimed. No language in the specification should be construed as any indicating any non-claimed element as essential to practice of the invention as used herein.

As used herein, the terms “hormone-dependent breast disorder,” “hormone-dependent reproductive tract disorder,” “hormone-dependent breast and reproductive tract disorder” each and collectively include, without limitation, any breast or reproductive tract (gynecologic) disorder that is related to or is sensitive to high estrogen or normal estrogen levels that need to be reduced, disorders with estrogen-receptor positive (ER+) and/or progesterone-receptor positive (PR+) disorders, for example, breast disorders, endometriosis, uterine fibroids (also called leiomyomas) etc. Reproductive tract disorders include endometrial, ovarian, cervical, uterus, vaginal, and vulvar cancers. The terms “estrogen-related disorder” and “estrogen-receptor related disorder” may be used interchangeably to refer to the foregoing hormone dependent disorders. The disorders may be presented primarily or secondarily to an underlying disease, for example, prostate cancer or other disorders such as liver diseases. Hormone-dependent breast and reproductive tract disorder include, for example, McCune-Albright syndrome, which is a disorder caused by a mutation in the GNAS gene affecting bones, skin, and several hormone-producing (endocrine) tissues, often resulting in abnormal scar-like (fibrous) tissue in their bones, a condition called polyostotic fibrous dysplasia, hyperthyroidism in individuals carrying such mutations, and in girls often resulting in precocious puberty.

As used herein, “breast disorder” means any aberration or a constellation of aberrations in the breast. Such aberration may be proliferative, non-proliferative, benign or malignant. Breast disorders include benign lesions of the breast (e.g., hyperplasia), increased breast density, gynecomastia, mastalgia, and breast cancer. Benign breast lesions include, but are not limited to, hyperplasia, atypia, ductal hyperplasia, lobular hyperplasia, atypical ductal hyperplasia (ADH), and atypical lobular hyperplasia (ALH). While not cancerous, ADH and ALH may be indicative of a predisposition for breast cancer.

Breast density is a breast disorder identified by visual techniques such as mammography and reflects increased fibroglandular tissue within the breast, i.e., overgrowth of stromal and epithelial cells in the breast. Breast density is classified in 4 classes-Class A, B, C and D-based on the degree of severity of the density. It is an independent risk factor for breast cancer. At least 23 states in the USA require physicians to inform subjects if they have dense breast(s). There is currently no treatment for dense breasts, although subjects are reminded to make healthy lifestyle choices and undergo regular mammograms to monitor changes in breast.

Gynecomastia is a common male breast condition reflecting increased hyperplasia of the breast tissue, including epithelial hyperplasia, with prevalence of asymptomatic gynecomastia of 60% to 90% in neonates, 50% to 60% in adolescents, and up to 70% in men aged 50 to 69 years (Therapeutics and Clinical Risk Management 2011:7, 145-148). Newborn gynecomastia usually resolves itself within 4 weeks of birth and at least half of adolescent males experience gynecomastia with typical onset of 13 to 14 years of age (Tanner stage 3 or 4). Gynecomastia has been proposed to be a risk factor for male breast cancer.

Further, gynecomastia often presents itself secondarily to an underlying disorder such as prostate cancer, cirrhosis and liver disease, male hypogonadism, hyperthyroidism, renal failure and in patients undergoing hemodialysis, Type I diabetes mellites, etc. Further, medications, such as anti-androgen medications or certain anti-psychotics, themselves have been reported to cause up to 25% of cases of gynecomastia and can be categorized by their hormone-like action. For example, the most common side effects attributed to bicalutamide, a nonsteroidal antiandrogen used for treatment of prostate cancer, are gynecomastia and breast pain.

As used herein, “breast cancer” means any malignant tumor of breast cells. Breast cancer may be at any stage of breast cancer, including stages of a pre-cancer, an early-stage cancer, a non-metastatic cancer, a pre-metastatic cancer, a locally advanced cancer, and a metastatic cancer. There are several types of breast cancer. Exemplary breast cancers include, but are not limited to, ductal carcinoma in situ (DCIS), lobular carcinoma in situ (LCIS), invasive (or infiltrating) lobular carcinoma (ILC), invasive (or infiltrating) ductal carcinoma (IDC), microinvasive breast carcinoma (MIC), inflammatory breast cancer, ER-positive (ER+) breast cancer, ER-negative (ER−) breast cancer, HER2+ breast cancer, triple negative breast cancer (TNBC), adenoid cystic (adenocystic) carcinoma, low-grade adenosquamatous carcinoma, medullary carcinoma, mucinous (or colloid) carcinoma, papillary carcinoma, tubular carcinoma, metaplastic carcinoma, or micropapillary carcinoma. A single breast cancer tumor can be a combination of these types or be a mixture of invasive and in situ cancer.

DCIS is the most common non-invasive breast cancer. It involves the cell lining of the breast ducts. In DCIS, the cells have not spread beyond the walls of the duct into the surrounding breast tissue. About 1 in 5 new breast cancer cases will be DCIS. LCIS is a pre-cancerous neoplasia. It may be indicative of a predisposition for invasive cancer. LCIS only accounts for about 15% of the in situ (ductal or lobular) breast cancers.

IDC is the most invasive breast cancer. As the name applies, it is a carcinoma that begins in the breast ducts and then invades the surrounding fatty tissue. About 8 to 10 invasive breast cancers are infiltrating ductal carcinomas. IDC is often treated by surgery to excise the cancerous tissue, and radiation therapy. In addition, chemotherapy combined with immunotherapy (e.g., tamoxifen and tratuzumab) is often used to treat IDC. If the tumor is larger than 4 cm, then a radical mastectomy may be performed.

ILC is a cancer that develops in the lobules of the breast and has invaded the surrounding tissue. About 1 in 10 invasive breast cancer is an ILC. ILC is treated by surgery to excise the cancerous tissue, and radiation therapy. In addition, chemotherapy and immunotherapy combination (e.g., tamoxifen and tratuzumab) is often used as an adjuvant therapy to treat ILC.

Inflammatory breast cancer accounts for about 1% to 3% of all breast cancers. In inflammatory breast cancer, cancer cells block lymph vessels in the skin, resulting in the beast turning red and feeling warm. The affected breast may become larger or firmer, tender, or itchy. Inflammatory breast cancer is treated with chemotherapy, immunotherapy, radiation therapy and in some cases, surgery.

Estrogen Receptor positive (ER+) breast cancer is characterized by the presence of estrogen receptors on the surface of the cancerous cells. Growth of ER+ cancer cells is associated with the availability of estrogen (hormone-dependent or hormone sensitive breast cancer). Approximately, 80% of all breast cancers are ER+ breast cancers. Treatment options for ER+ breast cancer include chemotherapeutic agents that block estrogen (e.g., tamoxifen).

### EXAMPLES

The invention is further illustrated by the following non-limiting examples.

### Example 1

### (Z)-Endoxifen Synthesis that Produces Novel Endoxifen Compositions

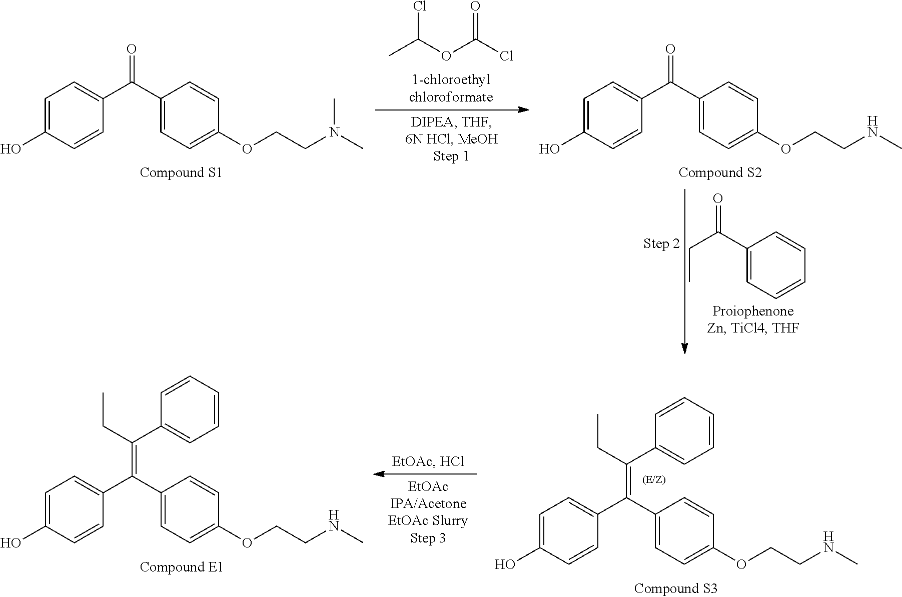

This example generally describes a synthetic route for (Z)-endoxifen (Compound E1) that produces other compounds to yield novel endoxifen compositions. The synthetic route is separated into three different synthetic steps below. Step 1 is a demethylation reaction of Compound S1 that results in Compound S2, as well as other compounds including: Compound 1, Compound 2, and Compound 3. Step 2 is a McMurry reaction coupling two carbonyl containing compounds (e.g., Compound S2 and propiophenone) together to form Compound S3, an E/Z mixture of endoxifen, as well as other compounds including Compound 4a, Compound 4b, Compound 5a, Compound 5b, Compound 6a, and Compound 6b. Step 3 is a recrystallization to produce an isomerically enriched mixture composition of (Z)-endoxifen (Compound E1), as well as Compound 7a.

### Step 1—Demethylation

This example describes the synthetic method for generating Compound S2. Compound S1 (1.0 equiv.) was added to tetrahydrofuran (THF) (9 vol., 7.9 wt.) in a suitable reactor. N-diisoethylpropylamine (DIEPA) (4.0 equiv., 1.8 wt.) was then added to the mixture over 30 minutes under an N₂ atmosphere. The mixture was then heated to 60° C. and 1-chloroethyl chloroformate (4.0 equiv., 2.0 wt.) was added to the mixture over 90 minutes. The mixture was then heated to reflux under stirring for 12 hours. A sample of the mixture was then tested by HPLC for reaction progress. If the HPLC result showed more than 1% of Compound S1, indicating residual starting material, an additional 0.23 wt. DIPEA and 0.25 wt. 1-chloroethyl chloroformate (di ACE-Cl) was added to the mixture and the mixture was held at reflux under stirring for an additional 2 hours. After the 12 hours of reflux (or 14 hours reflux if there was residual starting material), methanol (5 vol., 4 wt.) was added to the mixture over 2 hours at reflux and the mixture was held at reflux under stirring for an additional 30 minutes. After reflux, the mixture was distilled under partial vacuum to achieve a 5-volume residue. 6N HCl (4 wt.) was then added slowly over 2 hours to maintain an internal reaction temperature of 60° C. and the mixture was heated to reflux under stirring for 12 hours. A sample of the mixture was the tested by HPLC for impurities. If the HPLC showed more than 1% of a Compound 1, the mixture was further held at reflux under stirring for an additional 3 hours. After the second reflux was completed, the mixture was concentrated by applying a partial vacuum to achieve a 4-volume residue. Water (1 vol.) was then added to the mixture and the mixture was then distilled to achieve a 4-volume residue under full vacuum. The mixture was then cooled and 8N NaOH (at least about 5.0 vol., 5.0 wt.) was added slowly to the mixture until the mixture turned from a suspension to a dark brown solution with a pH greater or equal to 13. A sample of the solution was then tested by HPLC for side products. If the HPLC showed more than 1.5% of Compound 2, the mixture was heated to 60° C. and stirred for 2 hours and re-tested for Compound 2. Once the concentration of Compound 2 was below 1.5%, the mixture was then cooled, and ethyl acetate (4.0 vol., 3.6 wt.) was added for extraction. The mixture was stirred for 30 minutes and then left to settle under non-stirring conditions for 30 minutes. If an emulsion was observed at the interface, it was collected with the organic layer (e.g., ethyl acetate). The mixture was then separated into the organic layer and aqueous layer. The aqueous layer was kept under full vacuum for 30 minutes to remove any residual organic solvent. 6N HCl (0.6 vol., 0.6 wt.) was then added to the aqueous mixture until the pH was between 8-10. The aqueous mixture was then cooled over an hour to 0±5° C. and stirred for 2 hours. The aqueous mixture was then filtered and washed with purified water (3.0 vol., 3.0 wt.) to yield a wet solid cake. A solvent mixture of water:isopropyl alcohol (3:1 v/v) was then added to the wet solid cake and the mixture was stirred for 30 minutes at 40° C. and filtered again yielding a wet solid cake. The wet solid cake filtrate was then washed with ethyl acetate (2 vol., 1.8 wt.). The wet solid cake was then dried at 60° C. under vacuum to yield Compound S2. The yield of Compound S2, based on the amount of starting material of Compound S1, was no less than 70% yield. Compound 1, Compound 2, and Compound 3 also were formed during the Step 1 synthetic process and are further described in the following examples.

### Step 2—McMurry Reaction

In a dry vessel containing THF (10 vol., 8.9 wt., KF at most about 0.05%), zinc powder (0.96 wt.) was added under stirring at room temperature and N₂ flow. The vessel was then cooled to 0° C. and TiCl₄ (1.4 wt.) was added slowly to maintain an internal reaction temperature below 20° C. The solution was then heated to 60° C. and stirred for 2 hours. In a separate vessel, the carbonyl containing compounds (e.g., Compound S2 and propiophenone) were prepared by adding Compound S2 (1 equiv. wt.), synthesized as described in Step 1, and propiophenone (0.74 wt.) to THF (8 vol., 7.1 wt.) and heated to 60° C. The suspension of carbonyl containing compounds were then added to the first solution over a 1-hour time period and stirred for 8 hours at 60° C. A sample of the solution was then tested by HPLC for impurities. If the HPLC showed more than 1% of the starting material Compound S2, TiCl₄ (0.07 wt.) was added to the reaction and the reaction was further stirred for 2 hours at reflux temperature.

The reaction mixture was then distilled under vacuum until only 10 vol. remained. Then, MeTHF (10 vol.) was added to the distillate and distillation was re-performed until only 10 vol. remained. Another solvent addition of MeTHF (10 vol.) and the distillation was performed again until 10 vol. remained. During the distillation the internal reaction temperature did not exceed 40° C. To the 10 vol. distillate solution, 3N HCl (10 vol.) was added under stirring at 30° C. until gas release was observed. When gas is no longer being released, observed by the absence of bubbling, the solution was heated to 50° C. and stirred for 30 minutes. The solution was then filtered with a biphasic system and washed with MeTHF (1:1 w/w). The solution was then stirred for 15 minutes at 500 and left to undergo phase separation under non-stirring conditions for 30 minutes at 50° C. The aqueous phase was then discharged. The remaining organic phase was washed with 3N HCl (3 vol.) and stirred for 30 minutes at 50° C. and the aqueous phase was again discharged. This wash step was repeated once more. The organic phase was the cooled to 20-30° C. and 40% K₂CO₃ was added, and the solution was stirred for 1 hour after which a solid precipitate was observed. 30-35% HCl was added until the pH of the aqueous phase was 0-1 and the solid was completely dissolved. The solution was then heated to 50° C. and stirred for 30 minutes and then left to settle under non-stirring conditions for an additional 30 minutes. The aqueous phase was discharged, and the organic phase was washed with 1N NaOH (10 vol.) at 50° C. and stirred for 1 hour after which NaCl (1:1 w/w) was added to the solution and it was stirred for an additional 30 minutes at 50° C. and a solid precipitate was observed. The solution was then filtered, and the filtered precipitate (filter cake) was washed with MeTHF (1:1 w/w). The filter cake in MeTHF solution was then heated to 50° C. and stirred for 15 minutes and left to settle under non-stirring conditions for 30 minutes. The aqueous phase was then discharged from the flask and 1N NaOH (10 vol.) was added, and the solution was stirred at 50° C. for 15 minutes after which NaCl (1:1 w/w) was added and the solution was stirred for an additional 30 minutes and left to settle under non-stirring conditions for 30 minutes. The aqueous phase was discharged, and the organic phase was washed again. After the second washing, 20% NaCl was added to the organic phase and the solution was stirred for 30 minutes at 50° C. and left to settle under non-stirring conditions for 30 minutes at 50° C. The aqueous phase was discharged from the bottom of the flask. The organic phase was then distilled at 50° C. to 2 vol. and ACN (10 vol.) was added, and the solution was again distilled back to 2 vol. ACN (10 vol.) was again added to the distillate and the solution was distilled once more to 3 vol.

A sample of the distillate was then tested by GC for impurities. If the GC analysis showed more than 2% of the MeTHF, another distillation was performed. After GC analysis, acetone (3 vol.) was added to the distillate and the solution was heated at 50° C. under stirring for 1 hour. The suspension was then cooled down to 5° C. over a time period of 4 hours (cooling rate of 15° C. per hour). The suspension was then stirred at 5° C. for 8 hours. A solid precipitate was observed, and the solution was filtered. The filtered precipitate (filter cake) was then washed with a pre-cooled solvent mixture of ACN:acetone (1:1 v/v, total 2 vol.). The wet filter cake was then dried for 4 hours at 70° C. under vacuum to yield Compound S3. The expected yield of Compound S3, based on the amount of starting material of Compound S2, was no less than 30% yield. Compound 4a, Compound 4b, Compound 5a, Compound 5b, Compound 6a, and Compound 6b also were formed during the Step 2 synthetic process and are further described in the following examples.

### Step 3—Isomeric Enrichment

In a vessel containing ethyl acetate (10.0 L, 9.0 kg, 9.0 wt.), Compound S3 (E/Z endoxifen), was added. 6N HCl (4.0 L, 4.0 kg, 4.0 wt.) was then slowly added to the solution while maintaining an internal reaction temperature of 10° C. The mixture was then heated to 60±5° C. and stirred for 10 hours. A sample of the solution was then sampled for HPLC analysis to determine E/Z isomerization purity to ensure the enrichment of the Z-isomer (Compound E1) was no less than 46%. After HPLC analysis, 8N NaOH (8.0 L, 8.0 kg, 8.0 kg) was slowly added to the solution until the solution reached a pH of 12 while also maintaining an internal reaction temperature of 10° C. Once the solution pH was equal or greater than 12, ethyl acetate (5 vol.) was added to the solution under stirring and then the solution was left to settle under non-stirring conditions for 10 minutes at a temperature of 10° C. or lower. The aqueous phase was then discharged, and if an emulsion was present, the emulsion was kept with the organic phase. The organic phase was then heated to 20-30° C. A 20% NaCl solution (3.0 L, 3.0 kg, 3.0 vol, 3.0 wt.) was then added to the organic phase and the mixture was heated to 50-60° C. The mixture was stirred and left to settle under non-stirring conditions for 10 minutes. The aqueous phase was then discharged, and the organic phase was filtered through activated carbon cartridges and then washed with ethyl acetate (5.0 vol., 4.5 kg, 5 vol., 4.5 wt.). The solution was then filtered to remove any residue from the activated carbon cartridges and any residue was rinsed at 50-60° C. to avoid product precipitation. The filtrate was then concentrated under vacuum until the volume reached 3 vol. Then ethyl acetate (9 vol.) was added to the solution (12 vol. total).

Ethanol (EtOH) was then added to the solution according to the formula: EtOH (Kg)=[(6.25-GC value)/93.75]*(Solution Volume)*0.902 g/mL. A sample of the solution was then used for GC analysis to ensure any residual EtOH was between 5-7.5%. Once the solution was confirmed to have an EtOH concentration between 5-7.5%, the solution is cooled to −2° C. over 2 hours and maintained at −2° C. for 2 hours. The solution was then heated to 50° C. in 1 hour and stirred for 20 minutes and cooled down to 0° C. over 4 hours. After 3 hours of cooling, a sample of the mixture was taken for analysis of the E/Z isomerization ratio by HPLC. Once the E/Z ratio of Compound S3 was determined to be no more than 0.25 by HPLC analysis, the mixture was then filtered and washed with pre-cooled ethyl acetate (1.5 L, 1.5 kg, 1.5 vol., 1.3 wt.). The filtered wet cake was then dried at 90° C. to yield enriched Z-endoxifen. To maximize yield, the remaining filtrate was then concentrated, and IPA (10.0 L, 3 vol.) was added. The solution was then further concentrated. IPA (10.0 L, 3 vol.) was added again, and the mixture was concentrated until a volume of 5 vol. A sample of the solution was then taken for GC analysis to ensure the ethyl acetate concentration was below 1%. Once the ethyl acetate concentration was confirmed to be lower than 1%, the solution was further distilled to 2 vol. The distillate was the cooled to 25° C. and acetone (1.5 L) was added for a total solution volume of 3.5 vol. The solution was then heated for 1 hour at 50° C. and stirred for 2 hours. The solution was then cooled to 0° C. over 4 hours (cooling rate less than 15° C. per hour). The solution was then stirred at 0±5° C. for 6 hours. The mixture was then filtered and washed with pre-cooled 2-propanol (2.0 L, 1.6 kg, 2.0 vol., 1.6 wt.). The filtered wet cake was then dried at 80° C. to yield an enriched composition of (Z)-endoxifen (Compound E1). The composition of (Z)-endoxifen (Compound E1) (1 wt.) was then dissolved in ethyl acetate (10 vol., 9 wt.). The mixture was agitated at 25° C. for 24 hours. The mixture was then filtered and washed with pre-cooled ethyl acetate (2 vol., 1.8 wt.). The filtered wet cake was then dried at 80° C. to yield the enriched composition of (Z)-endoxifen (Compound E1). The expected yield of (Z)-endoxifen (Compound E1), based on the amount of starting material of Compound S3 added, was no less than 14% yield. Compounds 7a and 7b were also formed during the Step 3 synthetic process and is further described in a following example.

### Example 2

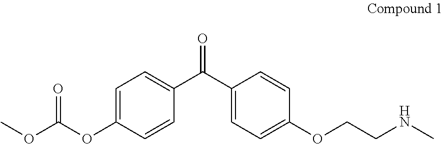

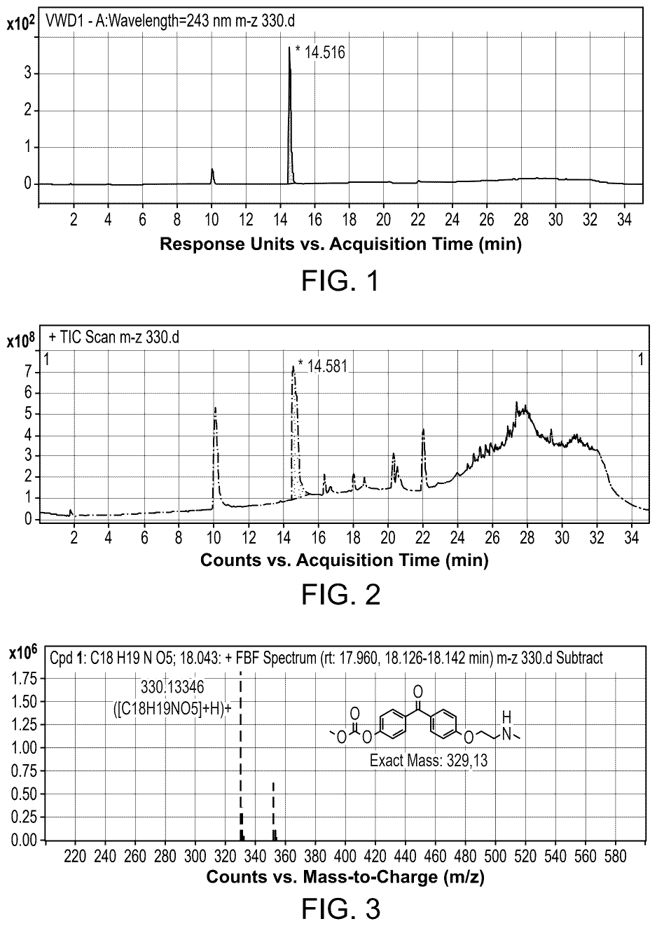

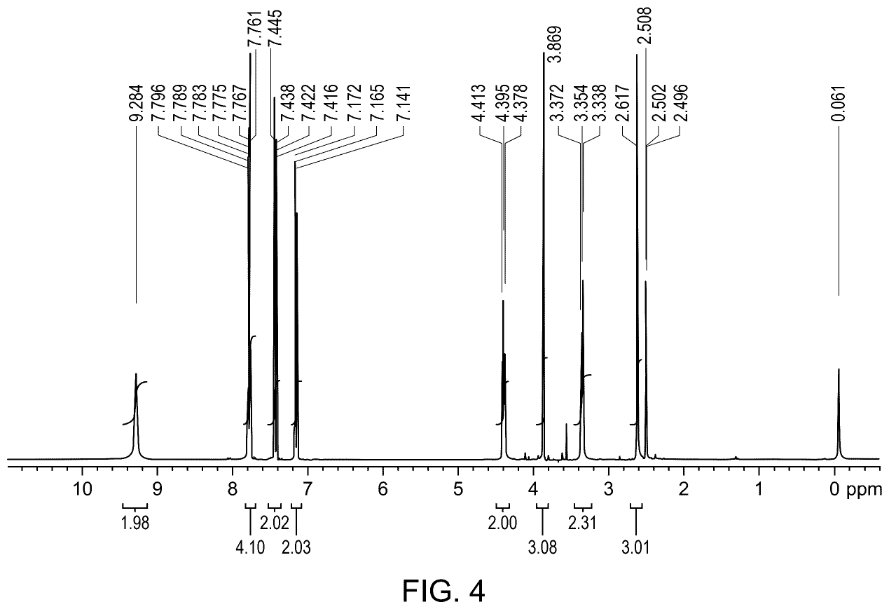

This example describes the synthesis and characterization of Compound 1. Compound 1 was generated during Step 1 of (Z)-endoxifen synthesis (as described in EXAMPLE 1), and was further characterized by UV, TIC, mass spectrometry, and NMR. The UV chromatogram of Compound 1 is shown in FIG. 1 and displays a peak of absorbance at 243 nm around 14 minutes. The TIC chromatogram of Compound 1 is shown in FIG. 2 and displays a peak at 14 minutes. The mass spectrum of Compound 1 is displayed in FIG. 3 and shows a peak at 330 m/z consistent with Compound 1. The proton NMR spectrum of Compound 1 is shown in FIG. 4 and the carbon NMR spectrum of Compound 1 is shown in FIG. 5.

### Example 3

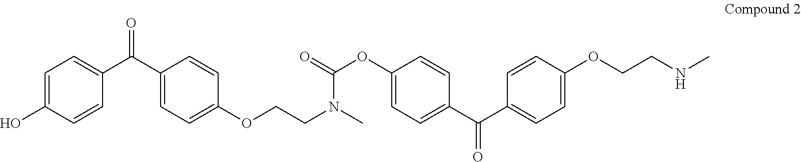 [SMILES: CNCCOc1ccc(C(=O)c2ccc(OC(=O)N(C)CCOc3ccc(C(=O)c4ccc(O)cc4)cc3)cc2)cc1]

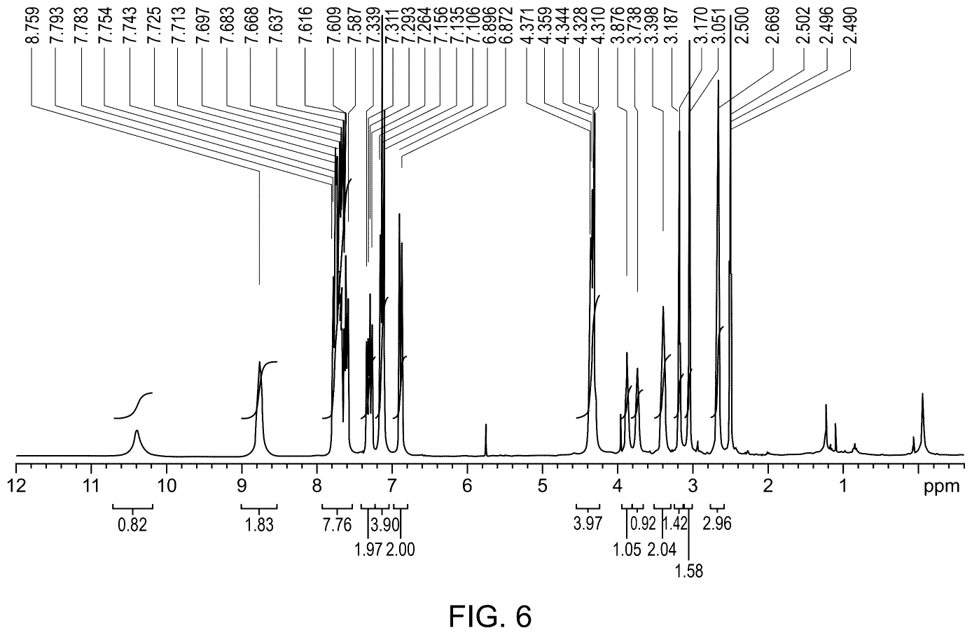

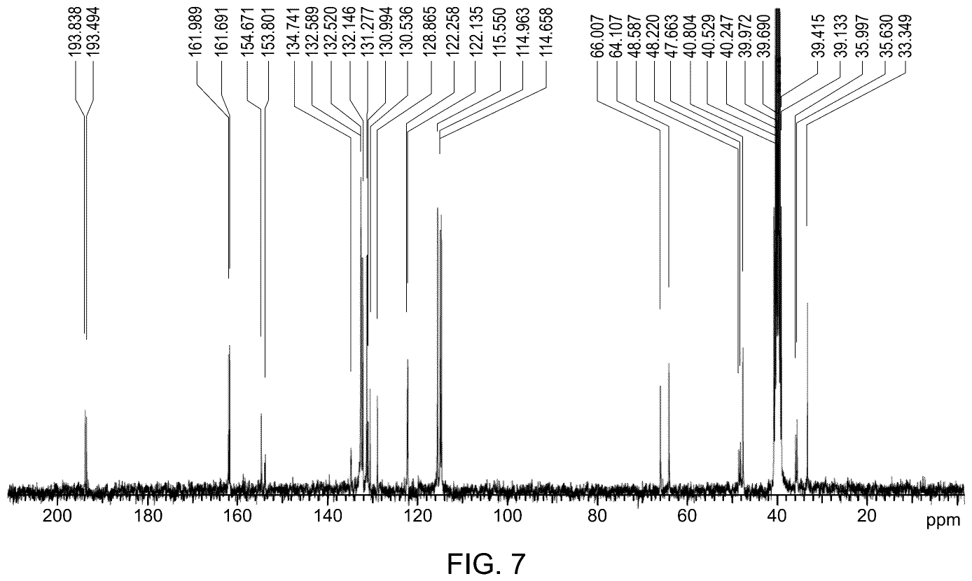

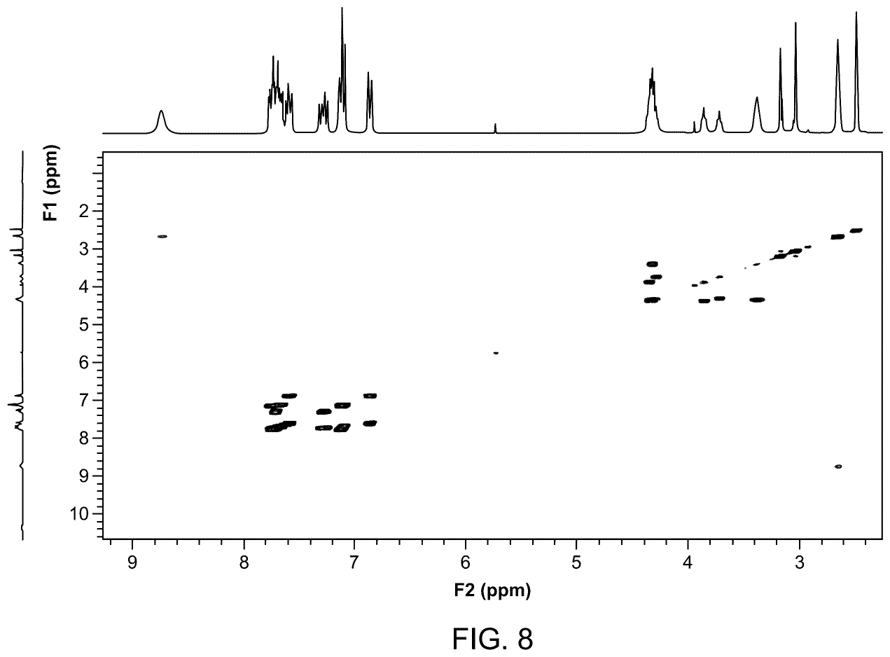

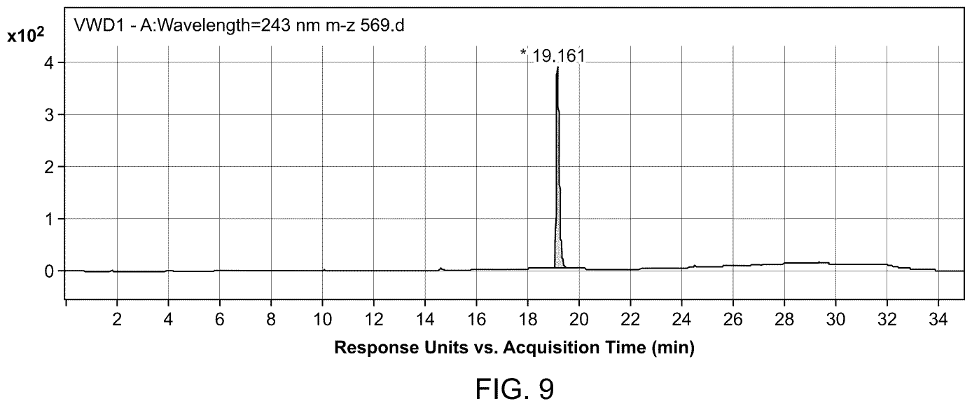

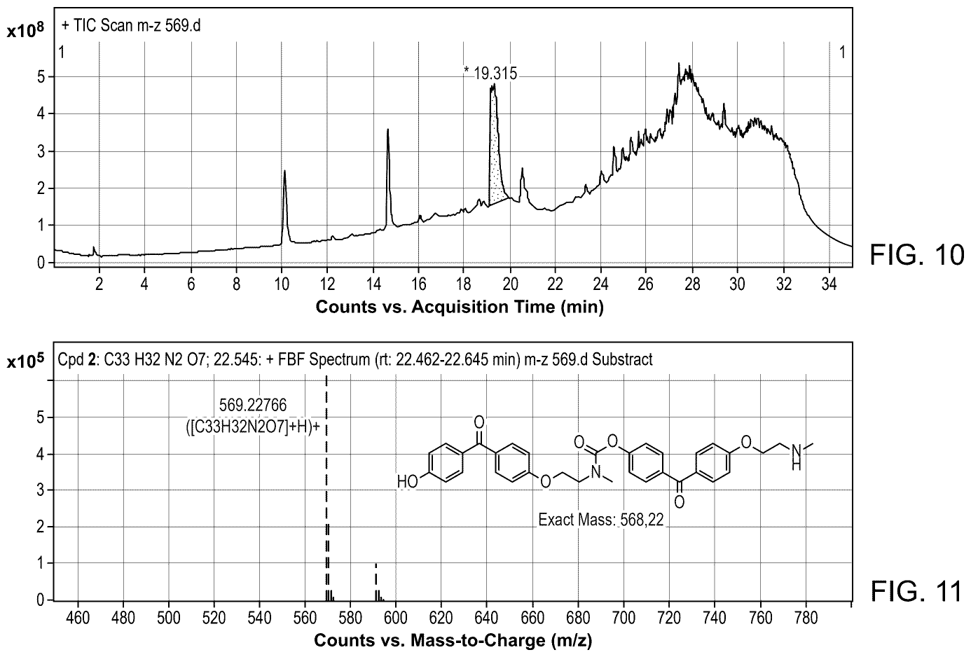

This example describes the synthesis and characterization of Compound 2. Compound 2 was generated during Step 1 of (Z)-endoxifen synthesis (as described in EXAMPLE 1), and was further characterized by NMR, correlated spectroscopy NMR (COSY NMR), UV, TIC, and mass spectrometry. The proton NMR spectrum of Compound 2 is displayed in FIG. 6, the carbon NMR spectrum of Compound 2 is shown in FIG. 7, and the COSY NMR 2D spectrum is shown in FIG. 8. The UV chromatogram of Compound 2 is shown in FIG. 9 and displays a peak of absorbance at 243 nm around 19 minutes. The TIC chromatogram of Compound 2 is shown in FIG. 10 and displays a peak of at 19 minutes. The mass spectrum of Compound 2 is shown in FIG. 11 and displays a peak at 569 m/z consistent with Compound 2.

### Example 4

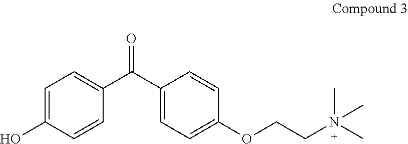

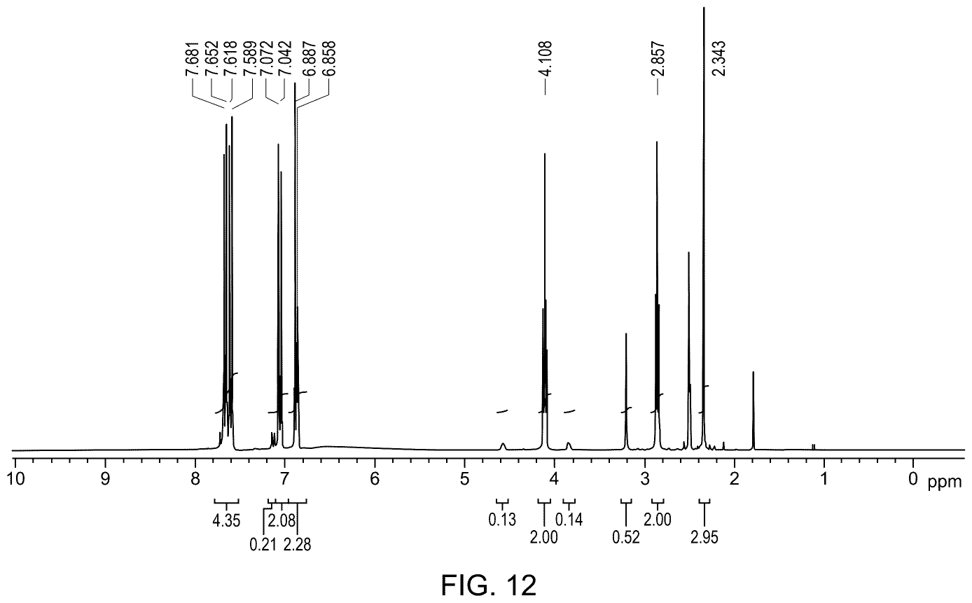

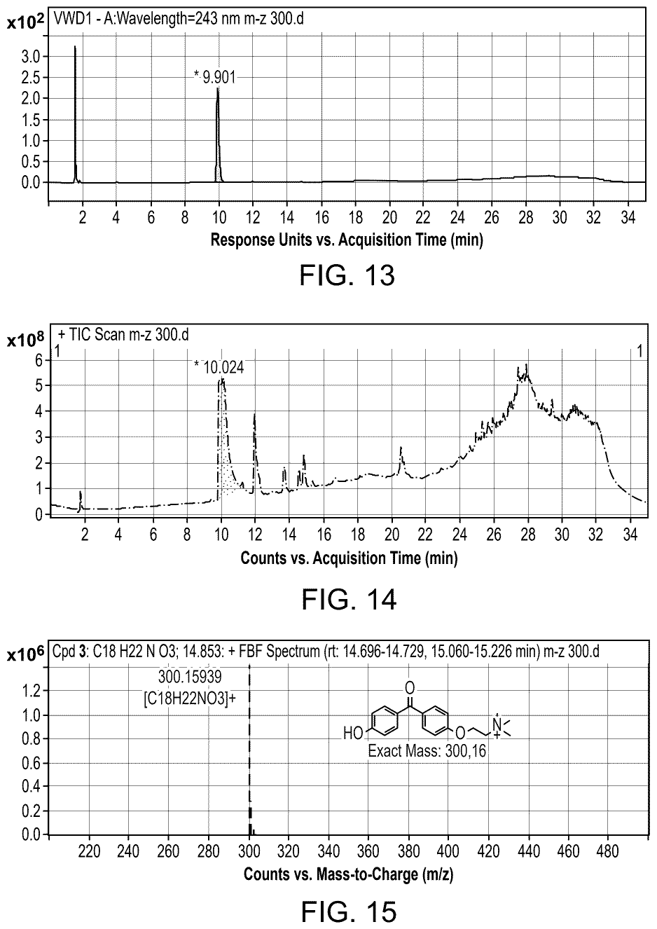

This example describes the synthesis and characterization of Compound 3. Compound 3 was generated during Step 1 of (Z)-endoxifen synthesis (as described in EXAMPLE 1), resulting from the methylation of unreacted starting material (Compound S1) and was further characterized by NMR, UV, TIC, and mass spectrometry. The proton NMR spectrum of Compound 3 is displayed in FIG. 12. The UV chromatogram of Compound 3 is displayed in FIG. 13 and shows a peak of absorbance at 243 nm around 10 minutes. The TIC chromatogram of Compound 3 is shown in FIG. 14 and shows a peak at 10 minutes. The mass spectrum of Compound 3 is shown in FIG. 15 and shows a peak at 300 m/z consistent with Compound 3.

### Example 5

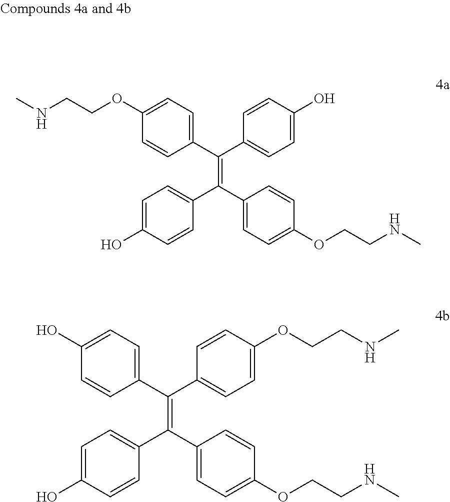

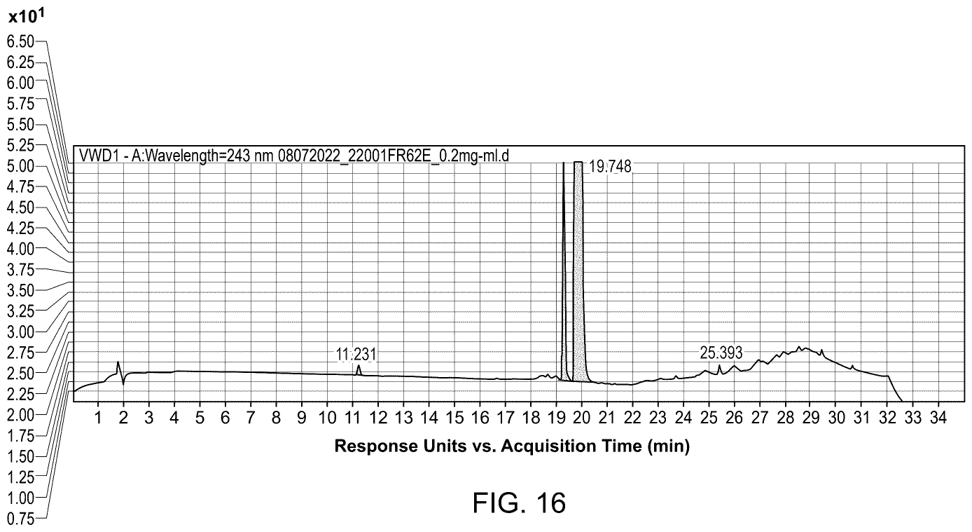

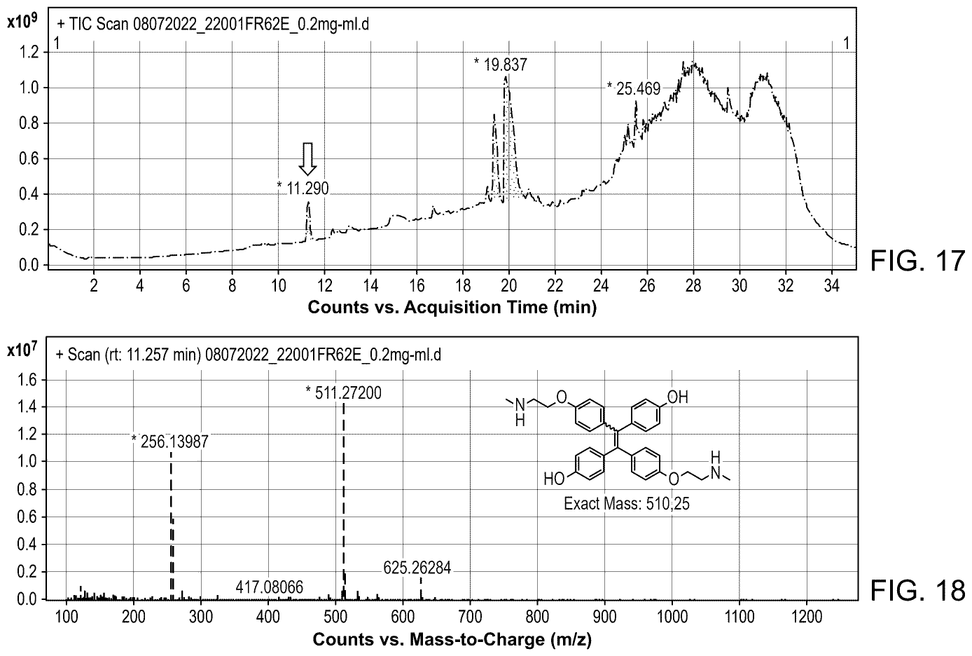

This example describes the synthesis and characterization of Compounds 4a and 4b. Compounds 4a and 4b were generated during Step 2 of (Z)-endoxifen synthesis (as described in EXAMPLE 1), by a homocoupling of Compound S2. Compounds 4a and 4b were characterized by UV, TIC, and mass spectrometry. The UV chromatogram of Compounds 4a and 4b is shown in FIG. 16 and displays a peak of absorbance at 243 nm at around 11 minutes. The TIC chromatogram of Compounds 4a and 4b is shown in FIG. 17 and displays a peak at 11 minutes. The mass spectrum of Compounds 4a and 4b is shown in FIG. 18 and has a peak at 511 m/z consistent with Compounds 4a and 4b.

### Example 6

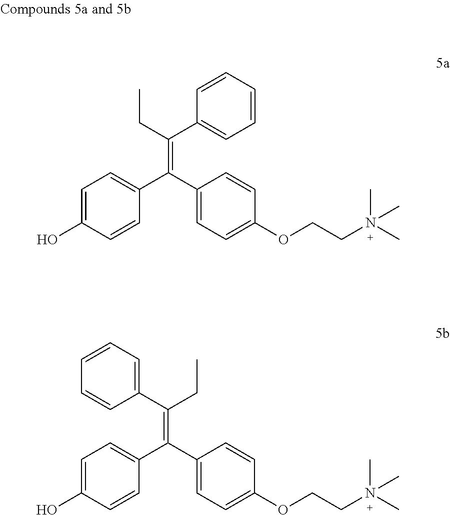

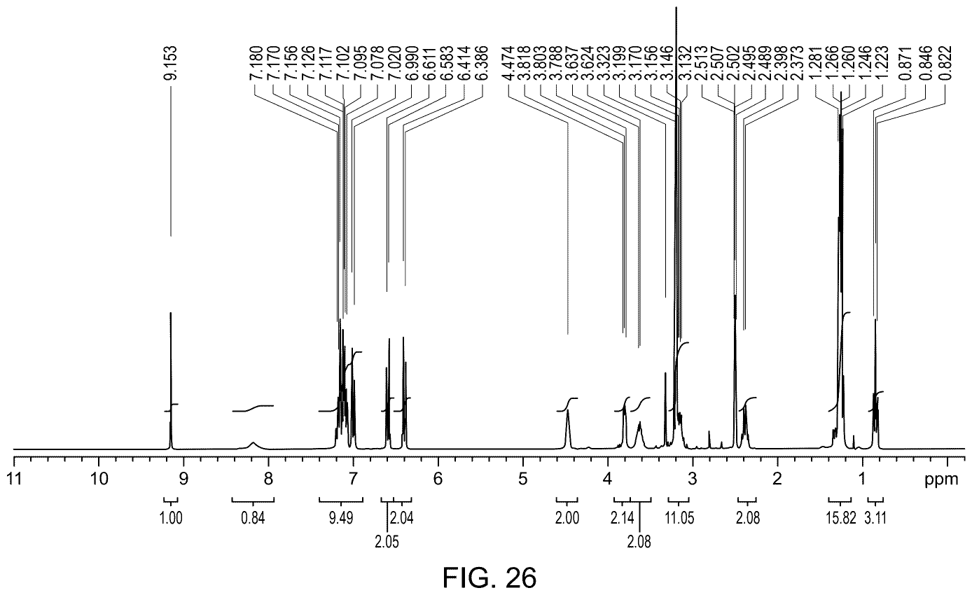

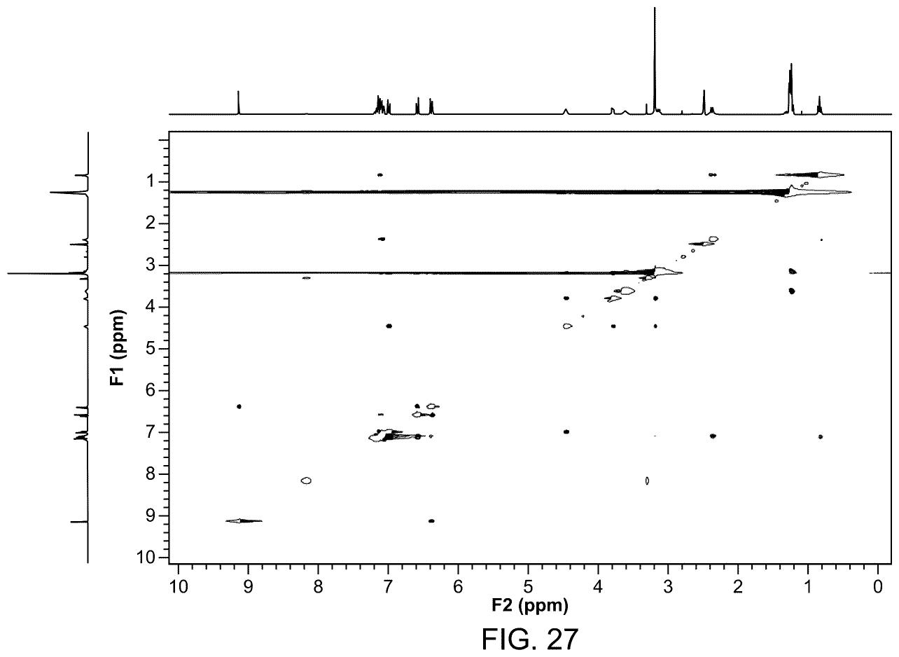

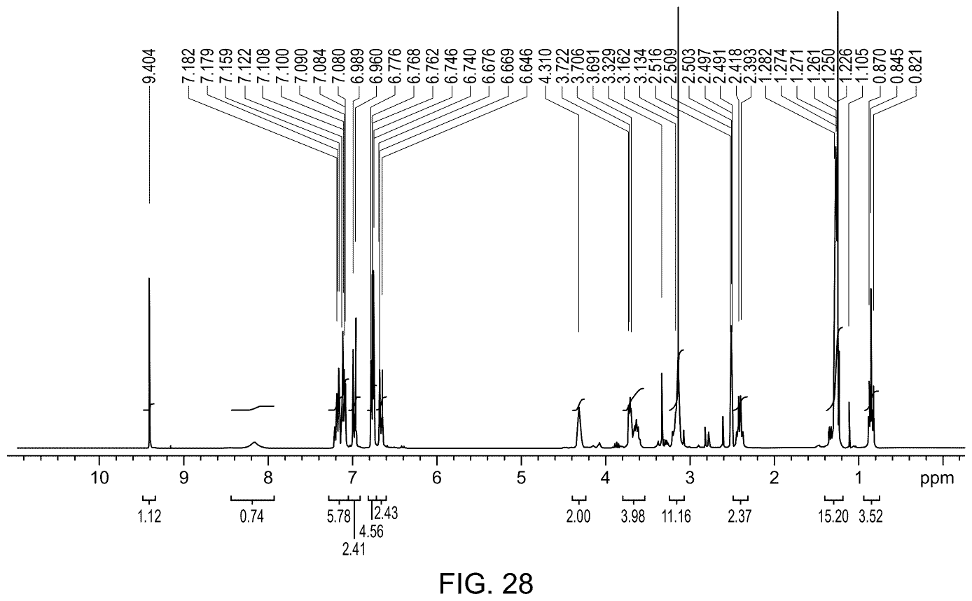

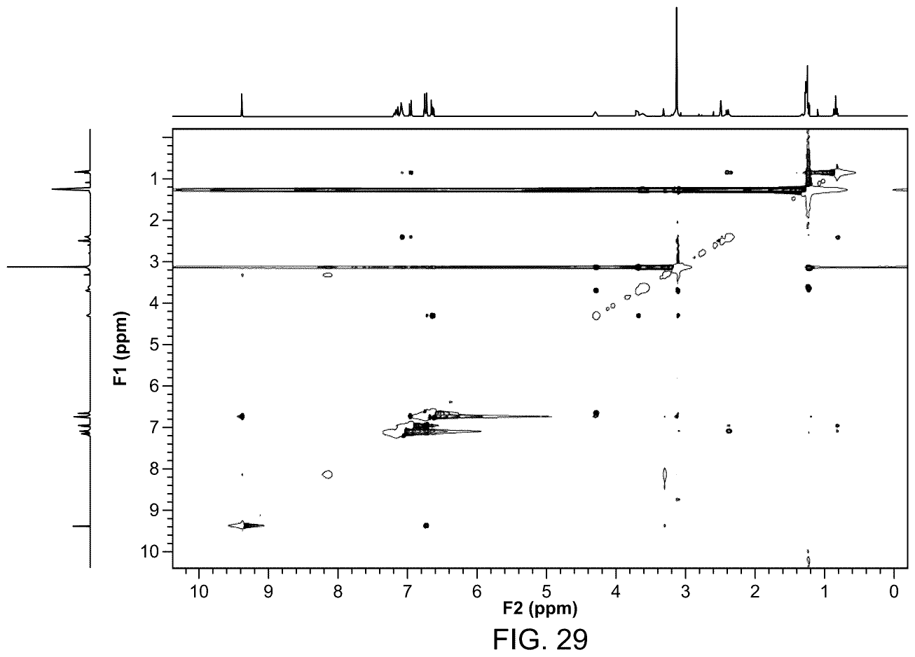

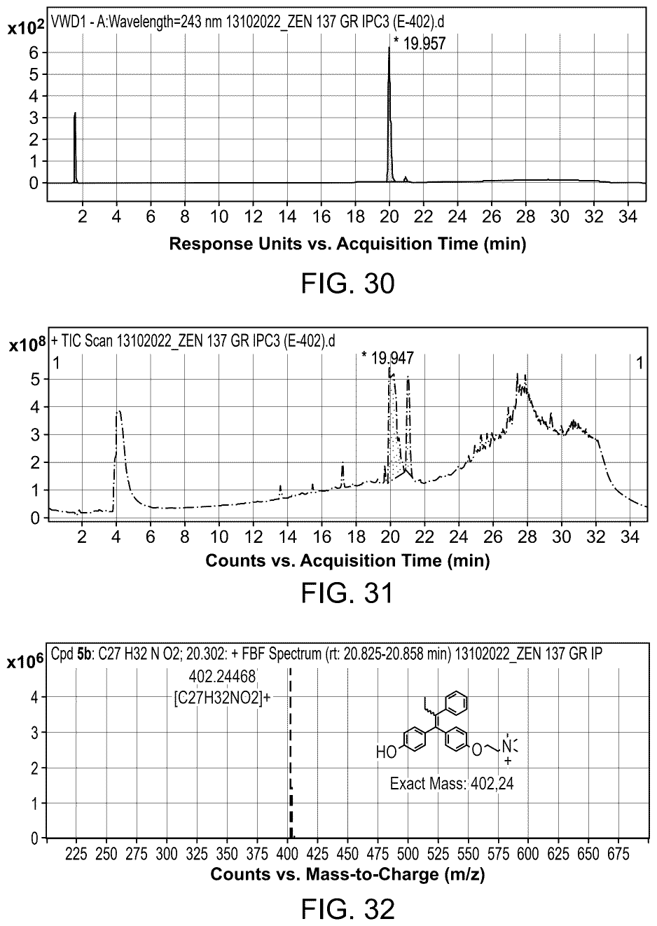

This example describes the synthesis and characterization of Compounds 5a. Compounds 5a and 5b were generated during Step 2 of (Z)-endoxifen synthesis (as described in EXAMPLE 1), by the coupling of Compound 3 and propiophenone. Compounds 5a and 5b were characterized by UV, TIC, mass spectrometry, NMR, and Rotating Frame Overhauser Enhancement Spectroscopy (ROESY). Compounds 5a and 5b was further purified to yield both the (E)-isomer, Compound 5b, and the (Z)-isomer, Compound 5a, that were both characterized by NMR, and Rotating Frame Overhauser Enhancement Spectroscopy (ROESY). The proton NMR of the (E)-isomer of Compound 5b is shown in FIG. 26, the ROESY spectrum of the (E) isomer of Compound 5b is shown in FIG. 27, the proton NMR of the (Z) isomer of Compound 5a is shown in FIG. 28, and the ROESY spectrum of the (Z) isomer of Compound 5a is shown in FIG. 29. The UV chromatogram of Compound 5b is shown in FIG. 30 and displays a peak of absorbance at 243 nm around 20 minutes. The TIC chromatogram of Compound 5b is shown in FIG. 31 and displays a peak at 20 minutes. The mass spectrum of Compound 5b is shown in FIG. 32 and has a peak at 402 m/z consistent with Compound 5b.

### Example 7

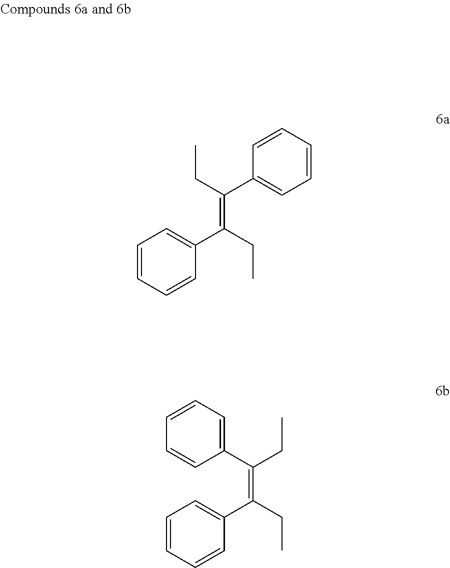

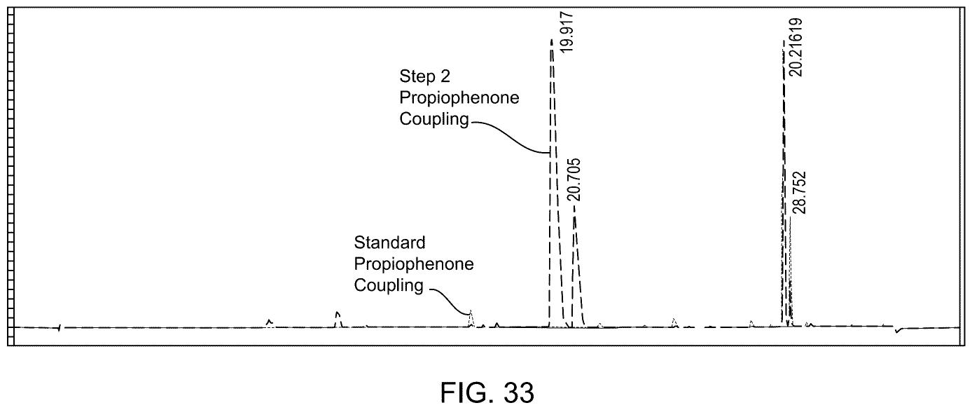

This example describes the synthesis and characterization of Compounds 6a and 6b. Compounds 6a and 6b were generated during Step 2 of (Z)-endoxifen synthesis (as described in EXAMPLE 1), from a homocoupling of 2 propiophenone molecules. The product of the propiophenone coupling lacks any ionizable groups and could not be characterized by mass spectrometry. Compounds 6a and 6b were analyzed by HPLC. Compounds 6a and 6b were compared against a reference chromatogram of a standard of Compounds 6a and 6b. As seen in FIG. 33, Compounds 6a and 6b had both (E) and (Z) isomers and overlayed well with the standard of Compounds 6a and 6b (Standard Propiophenone Coupling).

### Example 8

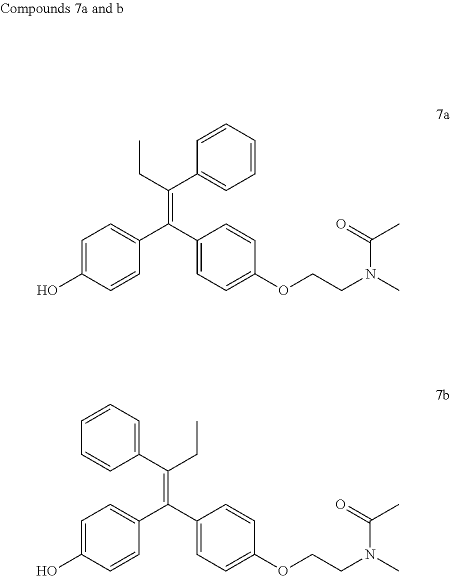 [SMILES: CC/C(=C(\c1ccc(O)cc1)c1ccc(OCCN(C)C(C)=O)cc1)c1ccccc1 ; CC/C(=C(/c1ccc(O)cc1)c1ccc(OCCN(C)C(C)=O)cc1)c1ccccc1]

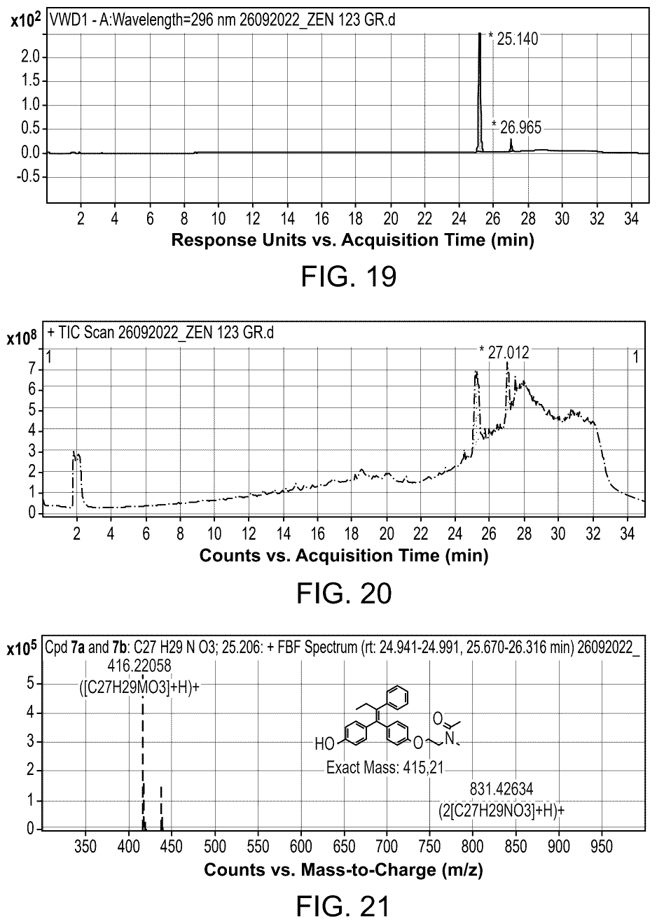

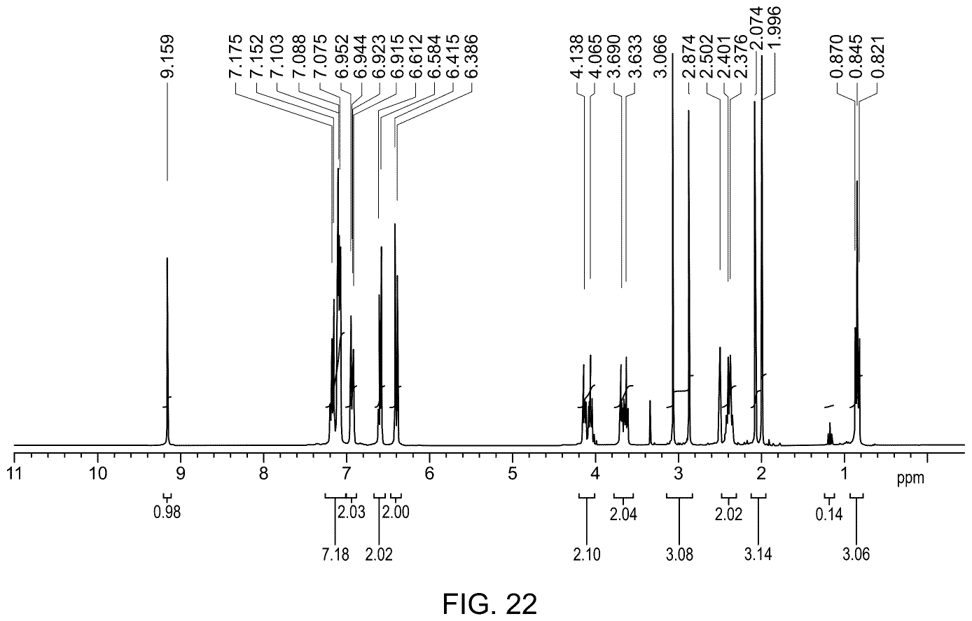

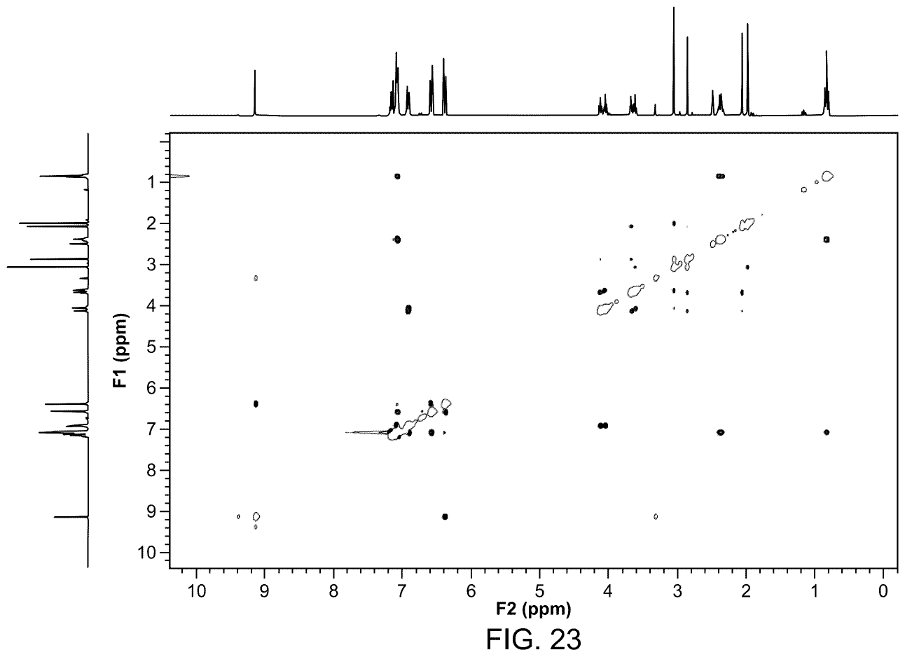

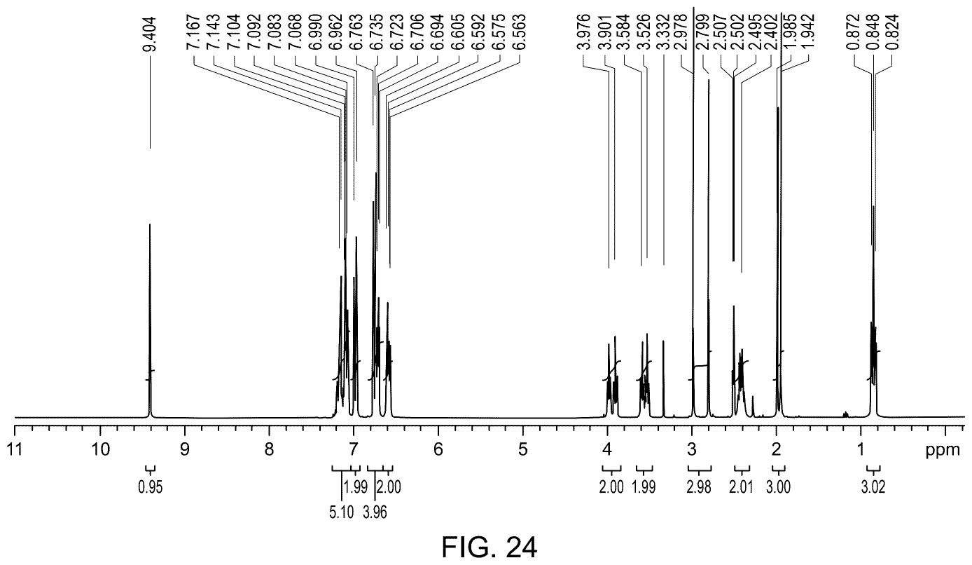

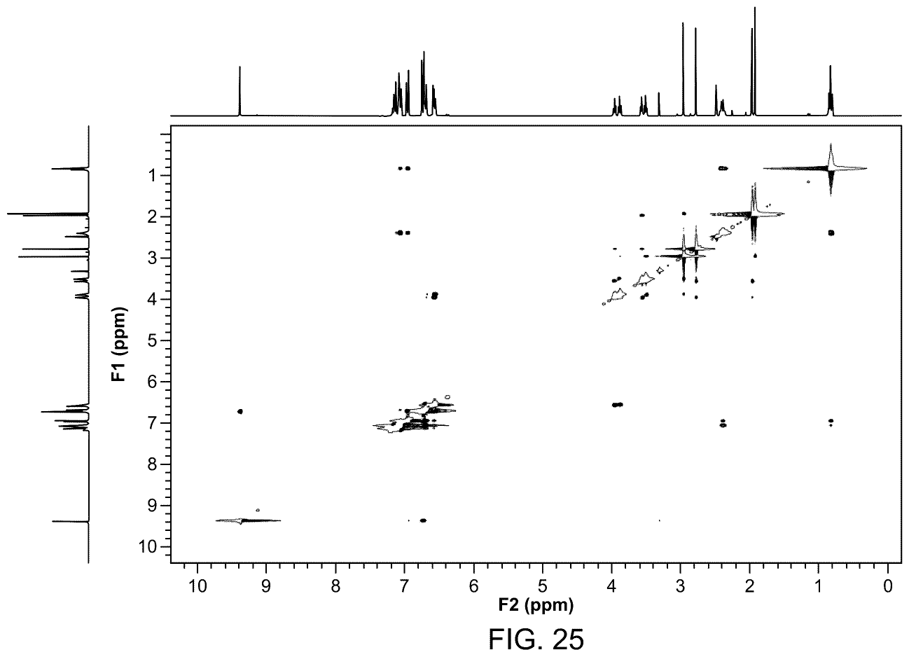

This example describes the synthesis and characterization of Compounds 7a and 7b. Compounds 7a and 7b were generated by acetylation of Compound S3 in the presence of ethyl acetate during Step 3 of (Z)-endoxifen synthesis (as described in EXAMPLE 1). Compounds 7a and 7b were formed and characterized by UV, TIC, and mass spectrometry. The UV chromatograph of Compound 7b is shown in FIG. 19 and displays a peak of absorbance at 296 nm around 25 minutes. The TIC chromatogram of Compound 7b is shown in FIG. 20 and displays a peak at 25 minutes, and 27 minutes. The mass spectrum of Compounds 7a and 7b is shown in FIG. 21 and shows a peak at 416 m/z consistent with Compounds 7a and 7b. Compounds 7a and 7b were further purified to yield both the (E)-isomer, Compound 7b, and the (Z)-isomer, Compound 7a, that were both characterized by NMR, and Rotating Frame Overhauser Enhancement Spectroscopy (ROESY). The proton NMR of the (E) isomer of Compound 7b is shown in FIG. 22, the ROESY spectrum of the (E) isomer of Compound 7b is shown in FIG. 23, the proton NMR of the (Z) isomer of Compound 7a is shown in FIG. 24, and the ROESY spectrum of the (Z) isomer of Compound 7a is shown in FIG. 25.

### Example 9

Treatment of a Patient with Breast Cancer with Endoxifen Compositions

This example describes treatment of breast cancer using an endoxifen composition. A patient having breast cancer is orally administered an endoxifen composition comprising any one of the following compounds: Compound E1, Compound S1, Compound S2, Compound S3, Compound 1, Compound 2, Compound 3, Compound 4a, Compound 4b, Compound 5a, Compound 5b, Compound 6a, Compound 6b, Compound 7a, Compound 7b, Compound 8, or a combination thereof, resulting from the synthetic process described in EXAMPLE 1-EXAMPLE 8, every day for up to 6 months, or until the breast cancer is treated. Oral administration of an endoxifen composition eliminates, slows the spread of, shrinks, or reduces symptoms of the breast cancer, thereby treating the breast cancer.

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Example 10

### Compound S2 Synthesis, DoE Screening

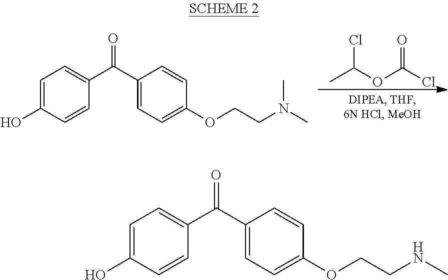

This example covers optimization of a method of synthesizing Compound S2 from Compound S1. The reaction was performed according to Step 1 of General Synthesis Method 2 as outlined in EXAMPLE 1. Compound S1

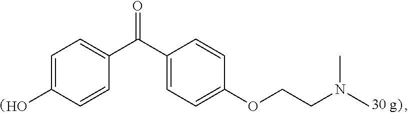 [SMILES: CN(C)CCOc1ccc(C(=O)c2ccc(O)cc2)cc1]

was combined with DIPEA in THF and was heated to 60° C. and ACE-Cl was added over 90-270 minutes. Methanol was then added. The mixture was stirred for 0.5-1.5 h and concentrated to 5V by distillation at atmospheric pressure. Then 6N HCl was added in 2 hours at 60° C. and stirred. Compound S2 was distilled at atmospheric pressure following completion of the reaction.

A DoE screen of the reaction was performed by varying 11 different conditions summarized in TABLE 2. Three center points for these reactions are summarized in TABLE 3. These data were analyzed by MiniTab software to reveal statistically significant parameters to be tested during a refining step. These analyses identified maintenance IPC 2 (10-20 h range), amount of 6 N HCl (3.2-4.8 w/w range), and dosing time of ACE-Cl (90-270 minute range) as important parameters for Compound S2 synthesis. The products from each run are summarized in TABLE 4.

TABLE 2

| | | | | | | | Dosing |
|---|---|---|---|---|---|---|---|
| | | | | | | Volumes | Time |
| | Time of | Amount | | Time of | Maintenance | of | ACE- |
| | Maintenance | of HCl, | ACE- | Maintenance | After | MeOH, | Cl, |
| Run | IPC 2, h | w/w | Cl eq. | IPCl, h | MeOH, v/w | v/w | Min |
| 1 | 15 | 4 | 2.5 | 8 | 1 | 5 | 180 |
| 2 | 20 | 3.2 | 4 | 12 | 1.5 | 4 | 90 |
| 3 | 10 | 3.2 | 4 | 4 | 1.5 | 6 | 270 |
| 4 | 20 | 4.8 | 2.5 | 4 | 1.5 | 4 | 270 |
| 5 | 10 | 4.8 | 4 | 12 | 0.5 | 4 | 270 |
| 6 | 10 | 4.8 | 2.5 | 12 | 1.5 | 6 | 90 |
| 7 | 10 | 3.2 | 2.5 | 4 | 0.5 | 4 | 90 |
| 8 | 20 | 4.8 | 4 | 4 | 0.5 | 6 | 90 |
| 9 | 15 | 4 | 2.5 | 8 | 1 | 5 | 180 |
| 10 | 20 | 3.2 | 2.5 | 12 | 0.5 | 6 | 270 |
| 11 | 15 | 4 | 2.5 | 8 | 1 | 5 | 180 |

TABLE 3

| | | Central | |
|---|---|---|---|
| Parameter | Low Level −1 | Point 0 | High Level +1 |
| Maintenance IPC 2, h | 10 | 15 | 20 |
| Quantity of 6N HCl (w/w) | 3.2 | 4 | 4.8 |
| ACE-Cl Equivalents | 1 | 2.5 | 4 |
| Maintenance IPC 1, h | 4 | 8 | 12 |
| Maintenance After Methanol | 0.5 | 1 | 1.5 |
| Addition, h | | | |
| Amount of Methanol (v/w) | 4 | 5 | 6 |
| Dosing Time of ACE-Cl, min | 90 | 180 | 270 |

TABLE 4

| | Compound | Compound | Compound | Compound |
|---|---|---|---|---|
| Run | S1, A % | 8, A % | 1, A % | S2, A % |
| 1 | 0 | 84.9 | 0.97 | 93 |
| 2 | 0 | 87.3 | 0.37 | 92.8 |
| 3 | 0 | 65.6 | 3 | 78.4 |
| 4 | 0 | 82.3 | 0.79 | 94 |
| 5 | 0 | 70.2 | 3.8 | 88.5 |
| 6 | 0 | 82.3 | 1.13 | 89.6 |
| 7 | 0 | 80.8 | 4.6 | 84.9 |
| 8 | 0 | 81.3 | 0.2 | 93.7 |
| 9 | 0.09 | 70.22 | 2.4 | 86.4 |
| 10 | 0.011 | 70.8 | 1.78 | 85.7 |
| 11 | 0.11 | 81.8 | 2 | 82.4 |

### Example 11

### Compound S3 DoE Screening

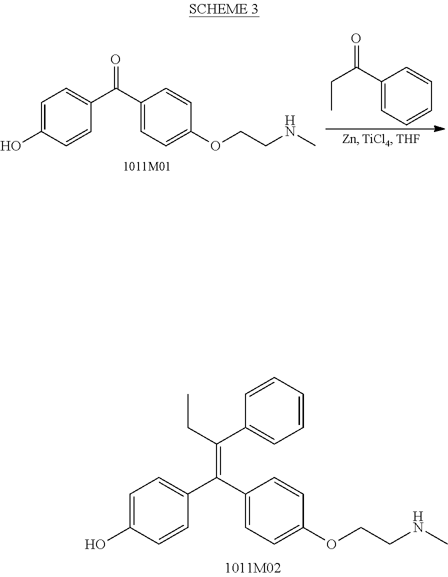

This example covers optimization of a method of synthesizing endoxifen from Compound S2. The reaction was performed according to Step 2 of General Synthesis Method 2 as outlined in EXAMPLE 1. A DoE screen of the reaction was performed by varying 11 different conditions as summarized in TABLE 5. Center points for these reactions are summarized in TABLE 6. Compound S2 input (20 g), KF of the THF (not more than 0.05%), the amount of THF (18 v), and reaction time (8 h) were fixed during this screening process. Results of these runs are summarized in TABLE 7. Complete conversion of Compound S2 was observed in reactions with 2 equivalents of TiCl₄ and 4 equivalents of Zn, indicating the criticality of these parameters.

TABLE 5

| | | | | | | T of |
|---|---|---|---|---|---|---|
| | | | Propiophenone | | Ketones | McMurry |
| Batch | TiCl₄ eq. | Zn eq. | eq. | KF, % | Addition, h | Reaction, ° C. |
| 1 | 2 | 2.4 | 1.5 | 0.5 | 1.5 | 66 |
| 2 | 2 | 4 | 1.5 | 0.1 | 3.5 | 45 |
| 3 | 1.6 | 3.2 | 1.75 | 0.3 | 2.5 | 55.5 |
| 4 | 1.6 | 4 | 1.5 | 0.5 | 3.5 | 66 |
| 5 | 2 | 2.4 | 2 | 0.5 | 3.5 | 45 |
| 6 | 1.6 | 3.2 | 1.75 | 0.3 | 2.5 | 55.5 |
| 7 | 1.6 | 3.2 | 1.75 | 0.3 | 2.5 | 55.5 |
| 8 | 1.6 | 2.4 | 1.5 | 0.1 | 1.5 | 45 |
| 9 | 1.6 | 2.4 | 2 | 0.1 | 3.5 | 66 |
| 10 | 1.6 | 4 | 2 | 0.5 | 1.5 | 45 |
| 11 | 2 | 4 | 2 | 0.1 | 1.5 | 66 |

TABLE 6

| | Low | Central | High |
|---|---|---|---|
| Parameter | Level −1 | Point 0 | Level +1 |
| TiCl₄ eq. | 1.2 | 1.6 | 2 |
| Zn eq. | 2.4 | 3.2 | 4 |
| Propiophenone eq. | 1.5 | 1.75 | 2 |
| KF of Compound | 0.1 | 0.3 | 0.5 |
| S2 in THF, % | | | |
| Time of Ketones | 1.5 | 2.5 | 3.5 |
| Addition, h | | | |
| T of McMurry | 45 | 55.5 | 66 |
| Reaction, ° C. | | | |

TABLE 7

| | | E + Z | m/z | Propiophenone |
|---|---|---|---|---|
| | Compound | Endoxifen, | 511, | Homocoupling, |
| Batch | S2, A % | A % | A % | A % |
| 1 | 23.25 | 71.7 | 2.8 | n/d |
| 2 | 0.49 | 89.26 | 7.9 | 1.28 |
| 3 | 23.2 | 69.8 | 2 | n/d |
| 4 | 27.8 | 66 | 0.1 | n/d |
| 5 | 46.1 | 49 | 1.9 | n/d |
| 6 | 25.2 | 60.6 | 2 | n/d |
| 7 | 34.8 | 59.6 | 25 | n/d |
| 8 | 24.8 | 70.4 | n/d | n/d |
| 9 | 48.2 | 45.6 | n/d | n/d |
| 10 | 11.7 | 82.9 | 0.3 | n/d |
| 11 | 0.12 | 94.4 | 2.7 | 1.93 |

### Example 12

### Optimization of (Z)-Endoxifen Production from a Mixture of (E)- and (Z)-Endoxifen

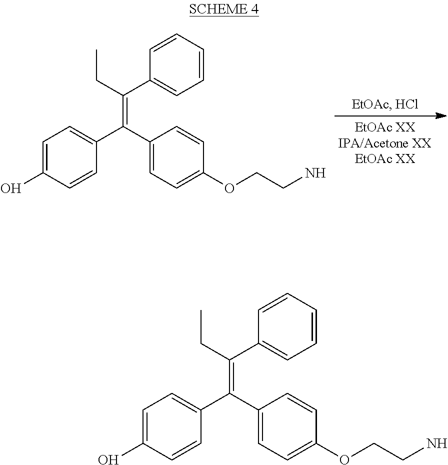 [SMILES: CC/C(=C(\c1ccc(O)cc1)c1ccc(OCCN)cc1)c1ccccc1 ; CC/C(=C(\c1ccc(O)cc1)c1ccc(OCCN)cc1)c1ccccc1]

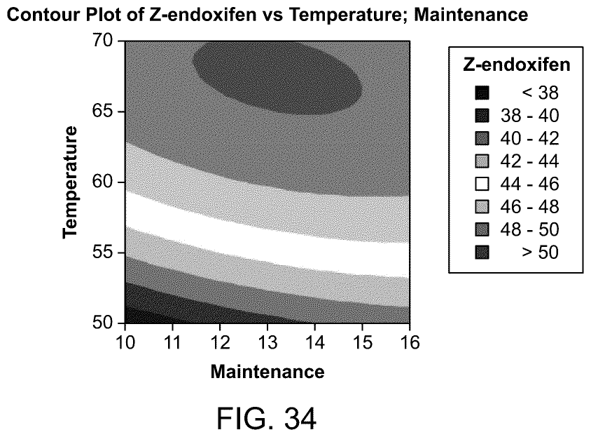

This example covers Z-endoxifen production from a mixture of E- and Z-endoxifen. The reaction was performed according to Step 3 of General Synthesis Method 2 as outlined in EXAMPLE 1. Briefly, this process was performed with F- and Z-endoxifen in ethyl acetate (9 w/w) in the presence of 6 N HCl (4 w/w). A DoE screen of the process was performed by varying maintenance IPC and temperature as summarized in TABLE 8. The test parameters for these reactions are summarized in TABLE 9. Results from the DoE screen are shown in the right column of TABLE 8. As shown in FIG. 34, the amount of Z-endoxifen product increased with temperature and decreased with maintenance time.

TABLE 8

| | | T of | IPC 1 |
|---|---|---|---|
| | Maintenance | Process, | Z Content |
| Run | IPC 1, h | ° C. | (Area %) |
| 1 | 10 | 50 | 35.7 |
| 2 | 13 | 60 | 48.2 |
| 3 | 13 | 60 | 48 |
| 4 | 13 | 60 | 47.8 |
| 5 | 10 | 70 | 49.4 |
| 6 | 16 | 70 | 50.4 |
| 7 | 16 | 50 | 41.5 |
| 8 | 13 | 60 | 51.2 |
| 9 | 10 | 60 | 47.0 |
| 10 | 13 | 60 | 48.8 |
| 11 | 16 | 60 | 46.3 |
| 12 | 13 | 50 | 39.0 |
| 13 | 13 | 60 | 46.4 |
| 14 | 13 | 70 | 48.8 |

TABLE 9

| | Low | Central | High |
|---|---|---|---|
| Parameter | Level −1 | Point 0 | Level +1 |
| Maintenance IPC 1, h | 10 | 13 | 16 |
| T of Process, ° C. | 50 | 60 | 70 |

### Example 13

### (Z)-Endoxifen Synthesis and Formulations

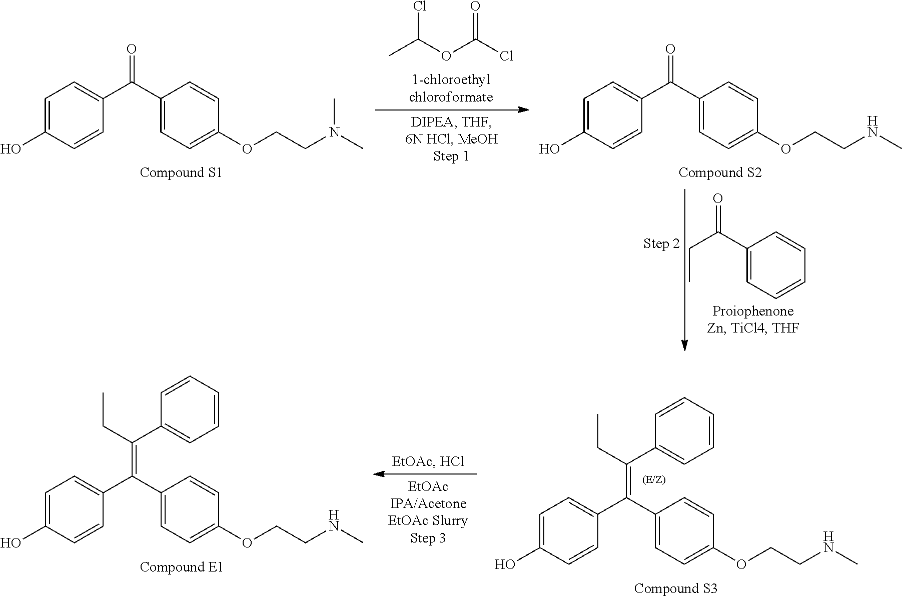

This example describes a synthetic route for (Z)-endoxifen (Compound E1) similar to the method described in EXAMPLE 1. The synthetic route included three different synthetic steps described in further detail below, namely demethylation of Compound S1 to form Compound S2, a McMurry reaction to form Compound S3 from Compound S2, and isometric enrichment of Compound S3 to form (Z)-endoxifen.

### Step 1—Demethylation

DIPEA was added to a suspension of Compound S2 in THF. The resultant mixture was then heated as chloroethyl chloroformate was added dropwise. After keeping the mixture in reflux for 12 hours, methanol was added and the suspension was concentrated to 5 v. Hydrochloric acid solution was added and the mixture was stirred at reflux for 12 hours. The mixture was distilled to 4 v and sodium hydroxide solution was added to adjust the pH. Next, phase separation was performed using ethyl acetate. Product precipitation was carried out by the addition of hydrochloric acid solution.

The product Compound S2 was isolated on a dryer-filter. The product was then slurried in deionized water followed by a deionized water/isopropanol mixture. Following a final wash with ethyl acetate, the product was dried and discharged. Three batches of Compound S2 product are summarized in TABLES 10-12 below.

TABLE 10
Compound S2 Batch 1

| Compound S1 Starting Material | 70 | kg |
|---|---|---|
| Compound S1 in final product | Not Detected | |
| m/z 330 Content (HPLC) | 0.9% | |
| m/z 569 Content (HPLC) | 0.6% | |
| m/z 300 Content (A/N %) | 0.1% | |
| Purity | 98.6% | |
| LOD | 5% | |
| Isolated Quantity of Compound S2 | 27.1 | kg |

TABLE 11
Compound S2 Batch 2

| | |
|---|---|
| Compound S1 Starting Material | 57.1 kg |
| Compound S1 in final product | Not Detected |
| m/z 330 Content (HPLC) | 0.9% |
| m/z 569 Content (HPLC) | 0.6% |
| m/z 300 Content (A/N %) | 0.1% |
| Purity | 98.6% |
| LOD | 5% |
| Isolated Quantity of Compound S2 | 27.1 kg |

TABLE 12
Compound S2 Batch 2 - Physical characteristics

| Test | Specification Limit | Result |
|---|---|---|
| Identification HPLC | Conforms to Standard | Compliant |
| Purity (HPLC-UV) | ≥97% | 98.6% |
| Loss on Drying | ≤5% | 5% |
| DIPEA | ≤1000 ppm | 20 ppm |

### Step 2—McMurry Reaction

Compound S2 and the starting material propiophenone were suspended in THF and then were transferred on the Zn/TiCl₄ suspension. The reaction mixture was refluxed for 8 h. At the completion of reaction, an exchange of solvent was carried out through a double addition and distillation of Me-THF. After the addition of hydrochloric acid solution and salt filtration, a phase separation was performed. The organic phase containing the intermediate Compound S3 was further washed twice with a HCl solution. Potassium carbonate solution was then added to the organic phase until it reached a basic pH. HCl solution was then added and a phase separation was performed. Sodium hydroxide solution was then added to the organic layer with subsequent addition of sodium chloride The biphasic system was then filtered. The aqueous phase was discharged and the organic phase was further washed twice with a mixture of sodium hydroxide and sodium chloride. Finally, the organic phase was washed with sodium chloride solution. The organic layer was then concentrated, followed by a solvent exchange using acetonitrile. Acetone was then added prior to product isolation. The suspension was cooled and the solid was isolated in filter-dryer. The wet solid was washed with acetonitrile and acetone. Finally, the product Compound S3 was dried and discharged.

### Step 3—Isomeric Enrichment

HCl solution was added to a cooled suspension of Compound S3 in ethyl acetate. The mixture was then heated and stirred for 10 h. Following this step, sodium hydroxide was added dropwise to the mixture as it was cooled. Phase separation was then performed with the addition of ethyl acetate. The organic layer was washed with NaCl solution and treated with charcoal. The solution was distilled and ethyl acetate and ethanol were added. A temperature ramp was carried out to isolate the isomer E of Endoxifen by filtration.

Next, a solvent exchange of mother liquors was performed with isopropanol. Acetone was then added, and the mixture was heated. Then, the mixture was cooled, stirred for 6 h, and then filtered. The wet solid was washed with isopropanol and dried, yielding the API (Z)-Endoxifen. In some cases, a further ethyl acetate slurry was performed.

### (Z)-Endoxifen Product

The three (Z)-Endoxifen batches were generated at scales of 1.050 kg, 5.500 kg, and 18.600 kg. Each of these three batches had greater than 98% purity on a dry basis as determined by HPLC. The E-endoxifen, total impurity, water, sulphated ash, methanol, ethanol, acetone, isopropyl alcohol, acetonitrile, ethyl acetate, tetrahydrofuran, 2-methyltetrahydrofuran, benzene, mesityl oxide, zinc, and titanium content of each composition is summarized in TABLES 13-15.

TABLE 13

| | Measured Value | |
|---|---|---|
| Batch Size | 18.600 | kg |
| Description | Compliant | |
| Identification (IR) | Positive | |
| Identification (HPLC) | Positive | |
| Identification (XRPD) | Compliant | |
| Assay (HPLC) on Dried Basis | 98.5% | |
| Purity (HPLC) of (Z)-Endoxifen | 97% | |
| (E)-Endoxifen | 3% | |
| Max Individual Impurity | 0.16% | |
| Total Impurities | 3% | |
| Water Content (KF) | 0.1% | |
| Sulphated Ash | 2.2% | |
| Methanol | 16 | ppm |
| Ethanol | 2 | ppm |
| Acetone | 576 | ppm |
| Isopropyl Alcohol | 2712 | ppm |
| Acetonitrile | 4 | ppm |
| Ethyl Acetate | 300 | ppm |
| Tetrahydrofuran | 2 | ppm |
| 2-Methyltetrahydrofuran | Not Detected | |
| Benzene | <2 | ppm |
| Mesityl Oxide | >25 | ppm |
| Zinc (Zn) | 3 | ppm |
| Titanium (Ti) | 9 | ppm |

TABLE 14

| | Measured Value | |
|---|---|---|
| Batch Size | 1.050 | kg |
| Description | Compliant | |
| Identification (IR) | Positive | |
| Identification (HPLC) | Positive | |
| Identification (XRPD) | Compliant | |
| Assay (HPLC) on Dried Basis | 99.2% | |
| Purity (HPLC) of (Z)-Endoxifen | 98% | |
| (E)-Endoxifen | 2% | |
| Max Individual Impurity | 0.09% | |
| Total Impurities | 2% | |
| Water Content (KF) | 0.2% | |
| Sulphated Ash | 2.2% | |
| Methanol | 12 | ppm |
| Ethanol | 6 | ppm |
| Acetone | 80 | ppm |
| Isopropyl Alcohol | 1375 | ppm |
| Acetonitrile | 3 | ppm |
| Ethyl Acetate | 2823 | ppm |
| Tetrahydrofuran | 6 | ppm |
| 2-Methyltetrahydrofuran | Not Detected | |
| Benzene | <2 | ppm |
| Mesityl Oxide | >25 | ppm |
| Zinc (Zn) | 24 | ppm |
| Titanium (Ti) | 112 | ppm |

TABLE 15

| | Measured Value | |
|---|---|---|
| Batch Size | 5.500 | kg |
| Description | Compliant | |
| Identification (IR) | Positive | |
| Identification (HPLC) | Positive | |
| Identification (XRPD) | Compliant | |
| Assay (HPLC) on Dried Basis | 101.8% | |
| Purity (HPLC) of (Z)-Endoxifen | 98% | |
| (E)-Endoxifen | 2% | |
| Max Individual Impurity | 0.09% | |
| Total Impurities | 2% | |
| Water Content (KF) | 0.2% | |
| Sulphated Ash | 0.4% | |
| Methanol | 9 | ppm |
| Ethanol | 6 | ppm |
| Acetone | 89 | ppm |
| Isopropyl Alcohol | 1829 | ppm |
| Acetonitrile | 3 | ppm |
| Ethyl Acetate | 2297 | ppm |
| Tetrahydrofuran | 8 | ppm |
| 2-Methyltetrahydrofuran | Not Detected | |
| Benzene | <2 | ppm |
| Mesityl Oxide | >25 | ppm |
| Zinc (Zn) | 8 | ppm |
| Titanium (Ti) | 40 | ppm |

### Example 14

### Ethyl Acetate Washes During (Z)-Endoxifen Purification

This example covers ethyl acetate wash steps during the drying protocol in Step 3 of (Z)-endoxifen synthesis. (Z)-endoxifen was synthesized according to EXAMPLE 1. The synthesis produced (Z)-endoxifen with a purity of 96.9%, a maximum individual impurity level 0.32%, 0.09% Compound 7a, Compound 7b, and 2.4% (E)-endoxifen. Two impurities were identified at RRT 1.42 (0.11%) and 0.57 (0.32%).

To address the presence of Compounds 7a and 7b, which is an acetylated form of endoxifen, a portion of product was subjected to further drying on the filter drier at room temperature with the agitator stopped and under nitrogen flow. This process increased the purity of the product, and in particular reduced the quantity of the impurity identified at RRT 0.57 to 0.03%. It is noted that this impurity at RRT 0,57 was observed in some Compound S3 intermediate, for example at about 0.07%. Subsequently, its content is reduced to 0.03% in IPC3 for the batch involved in the present deviation.

The identity of the impurity at RRT 0.57 was then interrogated, and was determined to be mesityl oxide, which has an RRT of 0.57. Ethyl acetate slurry was tested as a means for further limiting the concentration of this impurity. HPLC analysis on a wet sample showed that the content of mesityl oxide was reduced to 1.93% after the first slurry and 0.01% after the second slurry and GC analysis on the dry sample showed that the content of Mesityl Oxide was equal to 47 ppm.

A further assay utilizing ethyl acetate slurries generated a product with 0.008% mesityl oxide, 0.06% Compounds 7a and 7b, and 0.08% of the impurity at RRT 1.42. Based on the obtained results, a second pulping in ethyl acetate was performed to further purge these species. In particular, a dried product was pulped in ethyl acetate for 24 hours. Following this treatment, the product was partially dried and then left in the filter dryer at room temperature. The sample was observed to have 0.01% mesityl oxide, 0.11% impurity with RRT 1.42, and 0.08% Compounds 7a and 7b. These results suggest that ethyl acetate pulping diminishes mesityl oxide concentration without affecting the concentration of Compounds 7a and 7b or the impurity with RRT 1.42.

A further batch of (Z)-endoxifen with a yield of 5.500 kg was obtained and analyzed. This batch was produced loading 40 kg of a previously synthesized batch of Compound S2. The lower than predicted yield of this batch was determined to stem from loss of the product in the mother liquor during an ethyl acetate wash. It was determined that the quantity of the (E)-isomer of Endoxifen does not depend on the conduction of the reaction but whether the insulated solid has been washed on filter dryer with ethyl acetate. According to the mass balance result, about 9.4% of the product was present in the mother liquors. Since this is characterized by a low ratio of 0.18%, a high content of Z isomer in the filtered E isomer could be due to a retention of the mother liquors in the filtered panel. Additionally, poor filtration of ethyl acetate in the dryer filter may be linked to the presence of fine crystals in the crushed solid caused by the geometry of the dryer filter itself. For these reasons, it was decided to filter out the mother waters waste of ethyl acetate and the washings to recover the product (Z) Endoxifen. Specifically, sample was filtered of the mother liquors in the filter dryer and then during product drying. This method yielded 1.050 kg of (Z)-endoxifen. Since this product was recovered from mother liquors, yield was not calculable. However, analyses related to purity, XRPD, and residual solvent were performed on the dried product already unloaded, and were all determined to be compliant with manufacturing specifications. It was thus determined that the root cause of the low yield of the initial batch covered in this example was due to product loss during ethyl acetate washing, leading to a high content of Z isomer in the filtered E isomer. However, it was also determined that (Z)-endoxifen can be recovered from ethyl acetate mother liquor.

### Example 15

### Controlling Mesityl Oxide Production During (Z)-Endoxifen Synthesis

This example is directed to mesityl oxide production and removal during (Z)-endoxifen synthesis. Mesityl Oxide values >25 ppm were obtained in all three (Z)-endoxifen batches, higher than desired for formulation into pharmaceutical compositions. It has emerged in the literature that Mesityl Oxide can be generated by acetone in the presence of amino and amino acid fractions. Considering that Compound S3 and (Z)-endoxifen batches have been observed to contain amino fractions, the possibility that these species are responsible for mesityl oxide formation was interrogated.

Laboratory-scale experiments were conducted to test this hypothesis. From these analysis, it was found that the generation of mesityl oxide is directly related to both the presence of (Z)-endoxifen and the duration at which (Z)-endoxifen precursor reaction mixtures are maintained at 50° C. One means identified for reducing mesityl oxide concentration is to pulp (Z)-endoxifen product in THF/IPA.

### Example 16

### (Z)-Endoxifen Solubility In Dissolution Media

This example describes development of a dissolution assay to measure dissolution of endoxifen formulations. Since solubility of endoxifen decreases as pH becomes less acidic, as shown in EXAMPLE 1, additional experiments were performed to improve solubility of endoxifen in the pH 6.8 buffer solution. Polysorbate 80 (PS80), a nonionic surfactant, was added in varying concentrations to improve solubility and mimic an intestinal environment. Surfactants, such as polysorbate 80, create micelles in the medium that mimic the bile acid aggregates in the small intestine. These micelles facilitate the diffusion and transport of product into the medium.

TABLE 16

| | Solubility |
|---|---|
| Sample | (μg/mL) |
| (Z)-endoxifen in pH 6.8 Buffer (Control) | 8.14 |
| (Z)-endoxifen in pH 6.8 Buffer with 0.5% Polysorbate 80 | 58.31 |
| (Z)-endoxifen in pH 6.8 Buffer with 1.0% Polysorbate 80 | 62.15 |

As shown in TABLE 16, which lists the solubility of (Z)-Endoxifen at pH 6.8 with and without Polysorbate 80, the addition of polysorbate 80 improved the solubility of endoxifen at pH 6.8.

Based on this solubility data, a dissolution method was developed for testing dissolution of encapsulated endoxifen formulations. The improved dissolution assay was developed based on a combination of the US pharmacopoeia (USP) dissolution method and the Japanese pharmacopoeia (JP) dissolution method. The media used in each of these methods is summarized in TABLE 17, which compares USP and JP dissolution method media.

TABLE 17

| | Acid Medium | Buffer Medium |
|---|---|---|
| USP/EP | 750 mL of 0.1N hydrochloric acid | Add 250 mL of 0.20M tribasic sodium |
| | during 2 hours | phosphate during 45 minutes (or for the |
| | | specified time given in the individual |
| | | monograph) |
| JP | 2 g of sodium chloride in 700 mL of | Dissolve 1.70 g of potassium |
| | demineralized water. Add 7 mL of | dihydrogen phosphate and 1.775 g |
| | concentrated hydrochloric acid (Merk, | of disodium hydrogen phosphate |
| | 36% in water) under stirring. Complete | anhydrous in sufficient demineralized |
| | with demineralized water qsp 1000 mL. | water to make 1000 mL. |

The improved dissolution method also included either 0.500 or 0.75% polysorbate 80 in the buffer medium to mimic conditions of the intestine and increase endoxifen solubility. The improved dissolution method is summarized in TABLE 18, which lists dissolution method conditions.

TABLE 18

| Parameters | Acid Stage Conditions | | Buffer Stage Conditions | |
|---|---|---|---|---|
| Medium | 2 g of sodium chloride in 700 mL | | Dissolve 1.70 g of potassium | |
| | of demineralized water. Add 7 mL | | dihydrogen phosphate and 1.775 g | |
| | of concentrated hydrochloric acid | | of disodium hydrogen phosphate | |
| | (Merk, 36% in water) under | | anhydrous in sufficient | |
| | stirring. Complete with | | demineralized water and 0.75%¹ | |
| | demineralized water qsp 1000 mL. | | Polysorbate 80 to make 1000 mL. | |
| Volume | 1000 | mL | 1000 | mL |
| Apparatus | USP type II (Paddle) | | USP type II (Paddle) | |
| Sinkers | Yes | | Yes | |
| Rotational | 75 | rpm | 75 | rpm |

| | | |
|---|---|---|
| Speed | | |
| Pull Time | 30, 60, 90, and 120 | 15, 30, 45, 60, and 90 |
| (minutes)² | | |
| Temperature | 37 ± 0.5° C. | 37 ± 0.5° C. |

¹0.5% PS80 was tested and showed incomplete dissolution therefore, 0.75% PS80 was tested and achieved full dissolution. Hence, moving forward, 0.75% PS80 will be included in the buffer medium.
²The pull times in this table correspond to the pull times used during the development of the dissolution method. The pull times may be altered in the final dissolution method used to release the drug product.

Briefly, an encapsulated endoxifen formulation was weighted down with a sinker in a vessel and incubated at about 37° C. in 1000 mL of acid medium for 2 hours while mixing with a paddle. Samples were collected at 30, 60, 90, and 120 minutes to measure the amount of capsule and endoxifen formulation that had dissolved into the medium. After 2 hours, the acid medium was replaced with 1000 mL of buffer medium and incubated for an additional 1.5 hours. Samples were collected at 15, 30, 45, 60, and 90 minutes after buffer replacement.

Samples collected at each pull time were assayed using HPLC-UV to determine percent release of endoxifen over time. Chromatography was performed using a Waters Xselect CSH Phenyl-Hexyl 3.5 μm, 4.6 mm×150 mm column or equivalent with a flow rate of 0.8 mL/min, a run time of 18 minutes, and an injection volume of 80 μL. The column was held at about 50° C., and UV of the elution was measured at 243 nm. The autosampler was held at a temperature of 5° C. to ensure stability of the samples. A gradient between mobile phase A (10 mM ammonium formate in water) and mobile phase B (ammonium formate in methanol) was performed as shown in TABLE 19, which lists chromatography gradient conditions.

TABLE 19

| | Mobile | Mobile |
|---|---|---|
| Time | phase | phase |
| (min) | A (%) | B (%) |
| 0.0 | 70 | 30 |
| 2.0 | 70 | 30 |
| 2.5 | 30 | 70 |
| 12.5 | 30 | 70 |
| 12.6 | 10 | 90 |
| 15.0 | 10 | 90 |
| 15.1 | 70 | 30 |
| 18.0 | 70 | 30 |

This improved dissolution assay was used to measure dissolution of high dose (Z)-endoxifen formulations.

1.-139. (canceled)

140. A composition comprising:
an endoxifen: 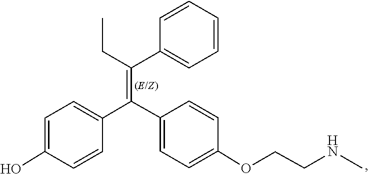 [SMILES: CC/C(=C(\c1ccc(O)cc1)c1ccc(OCCNC)cc1)c1ccccc1]
or a pharmaceutically acceptable salt thereof; and
not less than 0.01 wt % and not more than 1 wt % of 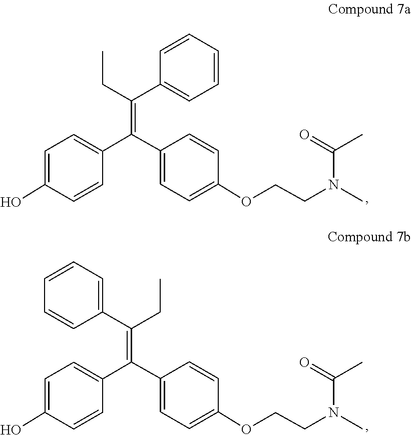 [SMILES: CC/C(=C(\c1ccc(O)cc1)c1ccc(OCCN(C)C(C)=O)cc1)c1ccccc1 ; CC/C(=C(/c1ccc(O)cc1)c1ccc(OCCN(C)C(C)=O)cc1)c1ccccc1]
or a combination thereof.

141. The composition of claim 140, wherein the composition further comprises not less than 0.01 wt % and not more than 1 wt % of a further component, wherein the further component comprises one or more of the following compounds, or a pharmaceutically acceptable salt of any one thereof:

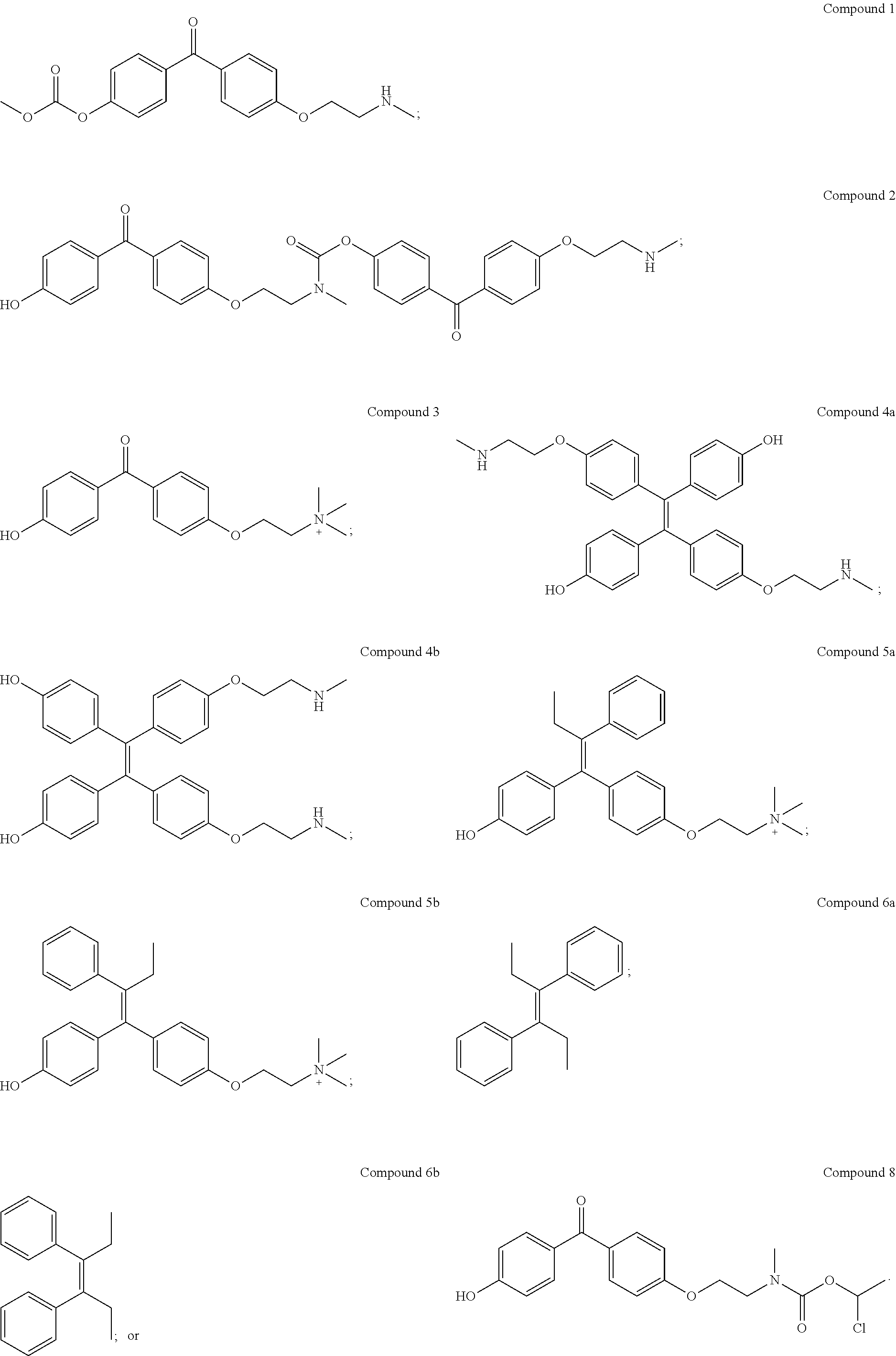

142. The composition of claim 140, wherein the endoxifen is not less than 95% (Z)-endoxifen:

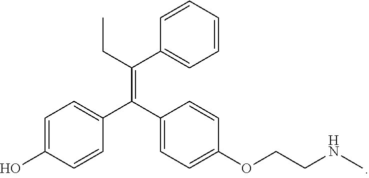 [SMILES: CC/C(=C(\c1ccc(O)cc1)c1ccc(OCCNC)cc1)c1ccccc1]

143. The composition of claim 140, wherein the endoxifen is not more than 2% (E)-endoxifen:

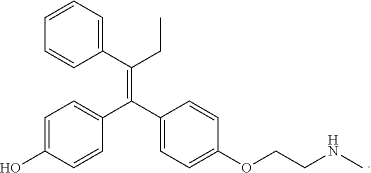 [SMILES: CC/C(=C(/c1ccc(O)cc1)c1ccc(OCCNC)cc1)c1ccccc1]

144. The composition of claim 140, wherein the composition comprises not less than not less than 0.01% and not more than 0.1% of Compound 7a and Compound 7b.

145. The composition of claim 141, wherein the composition comprises not less than 0.1% and not more than 1 wt % of Compound 1.

146. The composition of claim 141, wherein the composition comprises not less than 0.1% and not more than 1 wt % of Compound 2.

147. The composition of claim 141, wherein the composition comprises not less than 0.1% and not more than 1 wt % of Compound 3.

148. The composition of claim 141, wherein the composition comprises not less than not less than 0.1% and not more than 1% of Compound 4a and Compound 4b.

149. The composition of claim 141, wherein the composition comprises not less than not less than 0.01% and not more than 0.1% of Compound 5a and Compound 5b.

150. The composition of claim 141, wherein the total wt % of the one or more compounds is less than or equal to 2 wt %.

151. The composition of claim 140, wherein the composition has aromatase inhibition activity, an affinity to an estrogen receptor, or a combination thereof.

152. The composition of claim 140, wherein the pharmaceutically acceptable salt of the endoxifen is selected from the group consisting of an arecoline, besylate, bicarbonate, bitartarate, butylbromide, citrate, camysylate, gluconate, glutamate, glycollylarsanilate, hexylresorcinate, hydrabamine, hydrobromide, hydrochloride, hydroxynapthanoate, isethionate, malate, mandelate, mesylate, methylbromide, methylnitrate, methylsulfate, mucate, napsylate, nitrate, pamaoate (Embonate), pantothenate, phosphate/diphosphate, polygalacuronate, salicylate, stearate, sulfate, tannate, Teoclate, triethiodide, benzathine, clemizole, chloroprocaine, choline, diethylamine, diethanolamine, ethylenediamine, meglumine, piperazine, procaine, aluminum, barium, bismuth, lithium, magnesium, potassium, and zinc salt.

153. The composition of claim 152, wherein the pharmaceutically acceptable salt of the endoxifen is endoxifen gluconate, endoxifen citrate, or endoxifen hydrochloride.

154. The composition of claim 142, wherein the (Z)-endoxifen is polymorphic Form I of (Z)-endoxifen that is characterized by an x-ray powder diffraction pattern comprising major peaks at 16.8±0.3°, 17.1±0.3°, and 21.8±0.3° two theta.

155. The composition of claim 140, wherein the composition comprises from 0.01 mg to 200 mg of the endoxifen.

156. The composition of claim 140, wherein the composition comprises less than 0.25% water content.

157. The composition of claim 140, wherein the composition comprises less than:
6000 ppm methanol;
9000 ppm ethanol;
9000 ppm acetone;
9000 ppm isopropyl alcohol;
1000 ppm acetonitrile;
9000 ppm ethyl acetate;
1500 ppm tetrahydrofuran;
1500 ppm 2-methyltetrahydrofuran;
250 m benzene;
250 ppm mesityl oxide; or
any combinations thereof.

158. The composition of claim 140, wherein the composition comprises less than:
less than 500 ppm zinc;
less than 500 ppm titanium; or
a combination thereof.

159. The composition of claim 140, wherein the composition is formulated as a tablet or a capsule.

160. The composition of claim 159, wherein the formulation is an enteric formulation.

161. The composition of claim 159, wherein the formulation is a delayed-release formulation.

162. The composition of claim 159, wherein the formulation comprises hydroxypropyl methyl cellulose.

163. The composition of claim 140, wherein the composition is formulated as a pharmaceutical composition comprising the composition and a pharmaceutically acceptable excipient or diluent.

164. The pharmaceutical composition of claim 163, wherein the pharmaceutical composition is administered to a subject in need thereof.

165. The pharmaceutical composition of claim 164, wherein the subject in need thereof has or is at risk of having a hormone-dependent breast disorder, a hormone-dependent reproductive tract disorder, a depression, or any combinations thereof.

166. The pharmaceutical composition of claim 165, wherein the hormone-dependent breast disorder or the hormone-dependent reproductive tract disorder is a benign breast disorder, hyperplasia, atypia, atypical ductal hyperplasia, atypical lobular hyperplasia, increased breast density, gynecomastia, DCIS, LCIS, breast cancer, precocious puberty, McCune-Albright Syndrome, endometrial cancer, ovarian cancer, uterine cancer, cervical cancer, vaginal cancer, or vulvar cancer.

167. The pharmaceutical composition of claim 164, wherein the subject in need thereof has prostate cancer and has or is at risk of having gynecomastia.

168. The pharmaceutical composition of claim 164, wherein the subject in need thereof has tamoxifen-refractory or tamoxifen resistant hormone-dependent breast disorder or hormone-dependent reproductive tract disorder.

169. The pharmaceutical composition of claim 164, wherein the subject in need thereof is or will be treated with an SSRI drug selected from the group consisting of citalopram, escitalopram, fluoxetine, paroxetine, sertraline, and vilazodone.

170. The pharmaceutical composition of claim 164, wherein the subject in need thereof is administered the pharmaceutical composition daily.

171. The pharmaceutical composition of claim 163, wherein the pharmaceutical composition is used in a method of treating a hormone-dependent breast disorder, a hormone-dependent reproductive tract disorder, a depression, or any combinations thereof, the method comprising administration of an effective amount the pharmaceutical composition to a subject in need thereof, thereby treating the hormone-dependent breast disorder, the hormone-dependent reproductive tract disorder, a depression, or any combinations thereof.

172. The pharmaceutical composition of claim 171, wherein the hormone-dependent breast disorder or the hormone-dependent reproductive tract disorder is a benign breast disorder, hyperplasia, atypia, atypical ductal hyperplasia, atypical lobular hyperplasia, increased breast density, gynecomastia, DCIS, LCIS, breast cancer, precocious puberty, McCune-Albright Syndrome, endometrial cancer, ovarian cancer, uterine cancer, cervical cancer, vaginal cancer, or vulvar cancer.

173. The pharmaceutical composition of claim 171, wherein the subject in need thereof is tamoxifen-refractory or tamoxifen resistant.

174. The pharmaceutical composition of claim 171, wherein the subject in need thereof is administered the pharmaceutical composition daily.

175. A composition comprising:
an endoxifen: 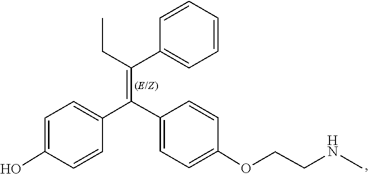 [SMILES: CC/C(=C(\c1ccc(O)cc1)c1ccc(OCCNC)cc1)c1ccccc1]
or a pharmaceutically acceptable salt thereof; and
not less than 0.01 wt % and not more than 1 wt % of 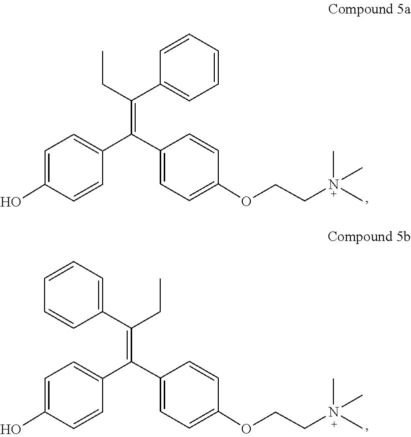
or
a combination thereof.

176. The composition of claim 175, wherein the composition further comprises not less than 0.01 wt % and not more than 1 wt % of a further component, wherein the further component comprises one or more of the following compounds, or a pharmaceutically acceptable salt of any one thereof:

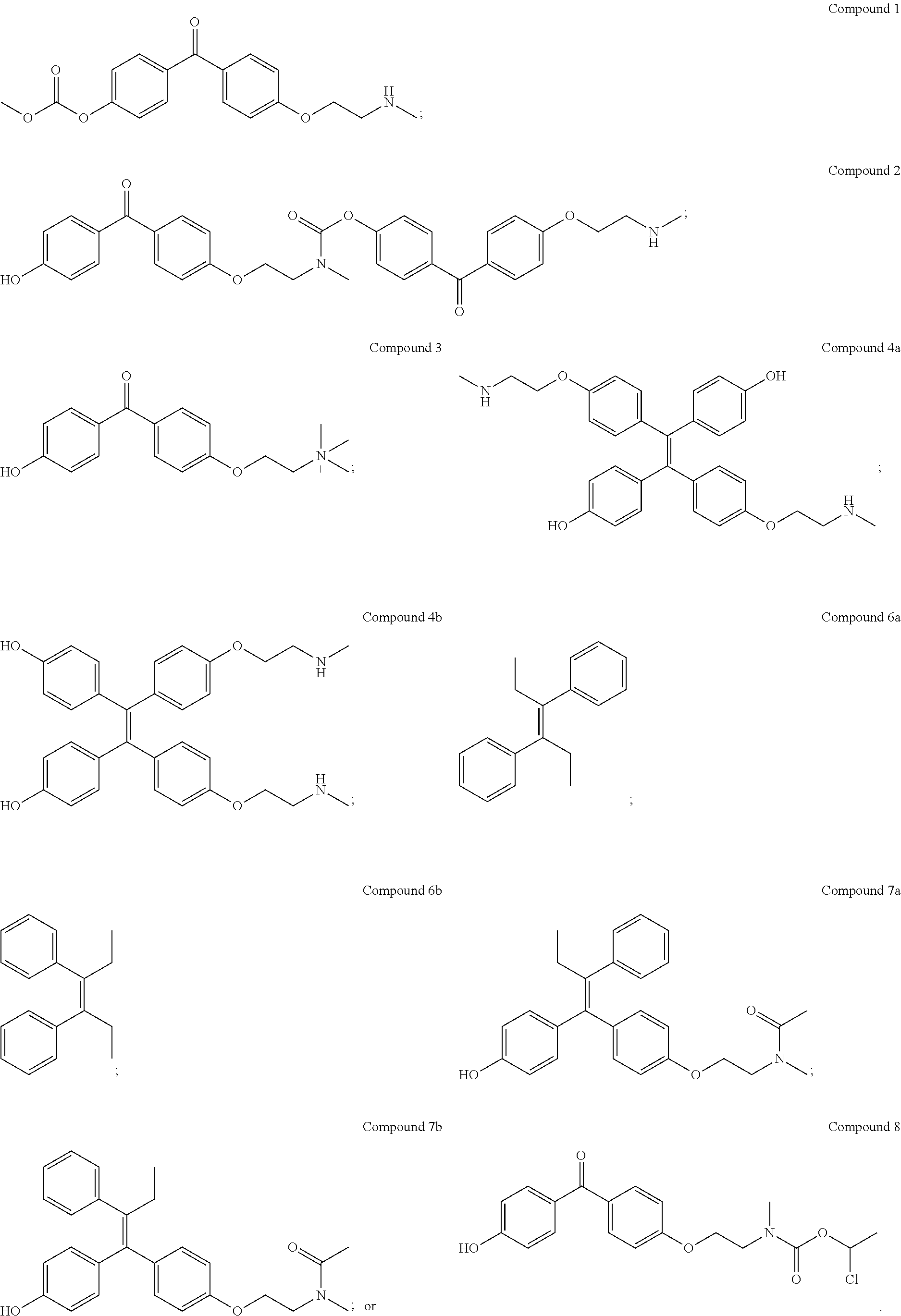

177. The composition of claim 175, wherein the endoxifen is not less than 95% (Z)-endoxifen:

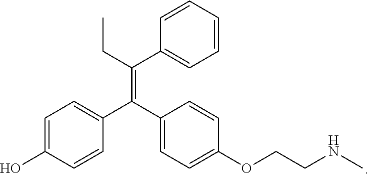 [SMILES: CC/C(=C(\c1ccc(O)cc1)c1ccc(OCCNC)cc1)c1ccccc1]

178. The composition of claim 175, wherein the endoxifen is not more than 2% (E)-endoxifen:

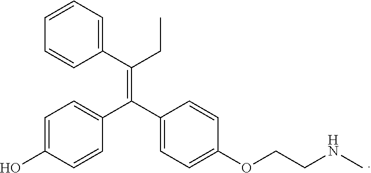 [SMILES: CC/C(=C(/c1ccc(O)cc1)c1ccc(OCCNC)cc1)c1ccccc1]

179. The composition of claim 175, wherein the composition comprises not less than not less than 0.01% and not more than 0.1% of Compound 5a and Compound 5b.

180. The composition of claim 176, wherein the composition comprises not less than 0.1% and not more than 1 wt % of Compound 1.

181. The composition of claim 176, wherein the composition comprises not less than 0.1% and not more than 1 wt % of Compound 2.

182. The composition of claim 176, wherein the composition comprises not less than 0.1% and not more than 1 wt % of Compound 3.

183. The composition of claim 176, wherein the composition comprises not less than not less than 0.1% and not more than 1% of Compound 4a and Compound 4b.

184. The composition of claim 176, wherein the composition comprises not less than not less than 0.01% and not more than 0.1% of Compound 7a and Compound 7b.

185. The composition of claim 176, wherein the total wt % of the one or more compounds is less than or equal to 2 wt %.

186. The composition of claim 175, wherein the composition has aromatase inhibition activity, an affinity to an estrogen receptor, or a combination thereof.

187. The composition of claim 175, wherein the pharmaceutically acceptable salt of the endoxifen is selected from the group consisting of an arecoline, besylate, bicarbonate, bitartarate, butylbromide, citrate, camysylate, gluconate, glutamate, glycollylarsanilate, hexylresorcinate, hydrabamine, hydrobromide, hydrochloride, hydroxynapthanoate, isethionate, malate, mandelate, mesylate, methylbromide, methylnitrate, methylsulfate, mucate, napsylate, nitrate, pamaoate (Embonate), pantothenate, phosphate/diphosphate, polygalacuronate, salicylate, stearate, sulfate, tannate, Teoclate, triethiodide, benzathine, clemizole, chloroprocaine, choline, diethylamine, diethanolamine, ethylenediamine, meglumine, piperazine, procaine, aluminum, barium, bismuth, lithium, magnesium, potassium, and zinc salt.

188. The composition of claim 187, wherein the pharmaceutically acceptable salt of the endoxifen is endoxifen gluconate, endoxifen citrate, or endoxifen hydrochloride.

189. The composition of claim 177, wherein the (Z)-endoxifen is polymorphic Form I of (Z)-endoxifen that is characterized by an x-ray powder diffraction pattern comprising major peaks at 16.8±0.3°, 17.1±0.3°, and 21.8±0.3° two theta.

190. The composition of claim 175, wherein the composition comprises from 0.01 mg to 200 mg of the endoxifen.

191. The composition of claim 175, wherein the composition comprises less than 0.25% water content.

192. The composition of claim 175, wherein the composition comprises less than:
6000 ppm methanol;
9000 ppm ethanol;
9000 ppm acetone;
9000 ppm isopropyl alcohol;
1000 ppm acetonitrile;
9000 ppm ethyl acetate;
1500 ppm tetrahydrofuran;
1500 ppm 2-methyltetrahydrofuran;
251 m benzene;
250 ppm mesityl oxide; or
any combinations thereof.

193. The composition of claim 175, wherein the composition comprises less than:
less than 500 ppm zinc;
less than 500 ppm titanium; or
a combination thereof.

194. The composition of claim 175, wherein the composition is formulated as a tablet or a capsule.

195. The composition of claim 194, wherein the formulation is an enteric formulation.

196. The composition of claim 194, wherein the formulation is a delayed-release formulation.

197. The composition of claim 194, wherein the formulation comprises hydroxypropyl methyl cellulose.

198. The composition of claim 175, wherein the composition is formulated as a pharmaceutical composition comprising the composition and a pharmaceutically acceptable excipient or diluent.

199. The pharmaceutical composition of claim 198, wherein the pharmaceutical composition is administered to a subject in need thereof.

200. The pharmaceutical composition of claim 199, wherein the subject in need thereof has or is at risk of having a hormone-dependent breast disorder, a hormone-dependent reproductive tract disorder, a depression, or any combinations thereof.

201. The pharmaceutical composition of claim 200, wherein the hormone-dependent breast disorder or the hormone-dependent reproductive tract disorder is a benign breast disorder, hyperplasia, atypia, atypical ductal hyperplasia, atypical lobular hyperplasia, increased breast density, gynecomastia, DCIS, LCIS, breast cancer, precocious puberty, McCune-Albright Syndrome, endometrial cancer, ovarian cancer, uterine cancer, cervical cancer, vaginal cancer, or vulvar cancer.

202. The pharmaceutical composition of claim 199, wherein the subject in need thereof has prostate cancer and has or is at risk of having gynecomastia.

203. The pharmaceutical composition of claim 199, wherein the subject in need thereof has tamoxifen-refractory or tamoxifen resistant hormone-dependent breast disorder or hormone-dependent reproductive tract disorder.

204. The pharmaceutical composition of claim 199, wherein the subject in need thereof is or will be treated with an SSRI drug selected from the group consisting of citalopram, escitalopram, fluoxetine, paroxetine, sertraline, and vilazodone.

205. The pharmaceutical composition of claim 199, wherein the subject in need thereof is administered the pharmaceutical composition daily.

206. The pharmaceutical composition of claim 198, wherein the pharmaceutical composition is used in a method of treating a hormone-dependent breast disorder, a hormone-dependent reproductive tract disorder, a depression, or any combinations thereof, the method comprising administration of an effective amount the pharmaceutical composition to a subject in need thereof, thereby treating the hormone-dependent breast disorder, the hormone-dependent reproductive tract disorder, a depression, or any combinations thereof.

207. The pharmaceutical composition of claim 206, wherein the hormone-dependent breast disorder or the hormone-dependent reproductive tract disorder is a benign breast disorder, hyperplasia, atypia, atypical ductal hyperplasia, atypical lobular hyperplasia, increased breast density, gynecomastia, DCIS, LCIS, breast cancer, precocious puberty, McCune-Albright Syndrome, endometrial cancer, ovarian cancer, uterine cancer, cervical cancer, vaginal cancer, or vulvar cancer.

208. The pharmaceutical composition of claim 206, wherein the subject in need thereof is tamoxifen-refractory or tamoxifen resistant.

209. The pharmaceutical composition of claim 206, wherein the subject in need thereof is administered the pharmaceutical composition daily.

210. A composition comprising:
an endoxifen: 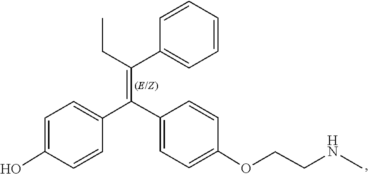 [SMILES: CC/C(=C(\c1ccc(O)cc1)c1ccc(OCCNC)cc1)c1ccccc1]
or a pharmaceutically acceptable salt thereof; and
not less than 0.01 wt % and not more than 1 wt % of 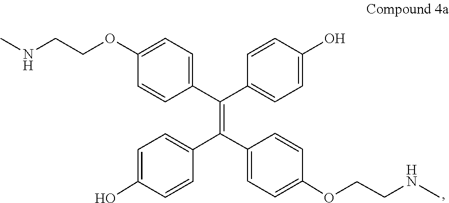
a combination thereof.

211. The composition of claim 210, wherein the composition further comprises not less than 0.01 wt % and not more than 1 wt % of a further component, wherein the further component comprises one or more of the following compounds, or a pharmaceutically acceptable salt of any one thereof:

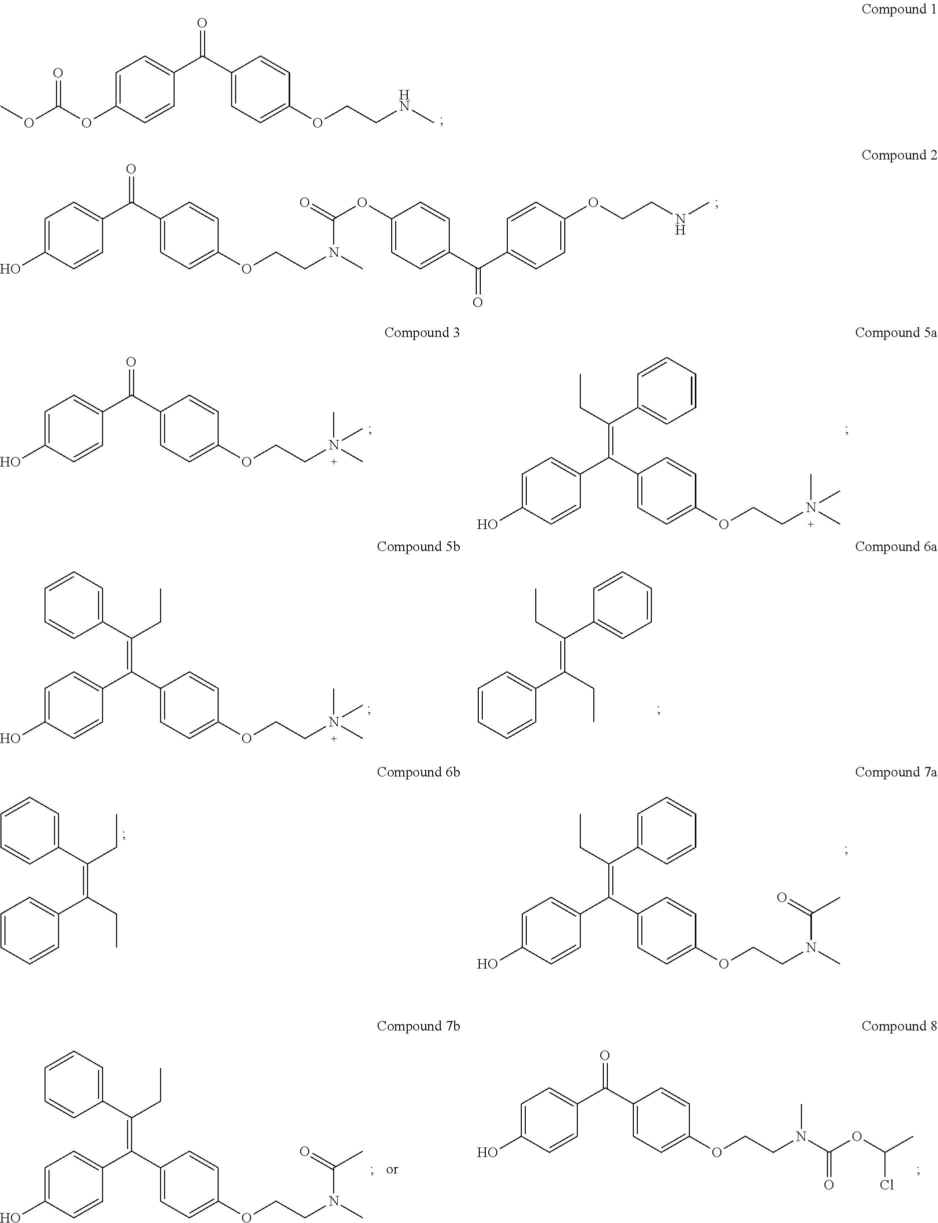

212. The composition of claim 210, wherein the endoxifen is not less than 95% (Z)-endoxifen:

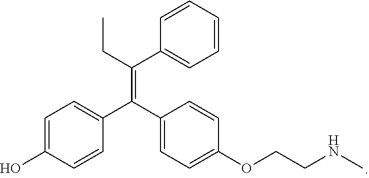 [SMILES: CC/C(=C(\c1ccc(O)cc1)c1ccc(OCCNC)cc1)c1ccccc1]

213. The composition of claim 210, wherein the endoxifen is not more than 2% (E)-endoxifen:

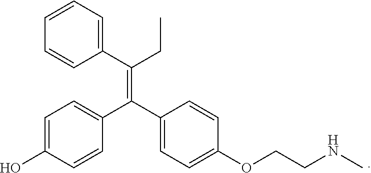 [SMILES: CC/C(=C(/c1ccc(O)cc1)c1ccc(OCCNC)cc1)c1ccccc1]

214. The composition of claim 210, wherein the composition comprises not less than not less than 0.1% and not more than 1% of Compound 4a and Compound 4b.

215. The composition of claim 211, wherein the composition comprises not less than 0.1% and not more than 1 wt % of Compound 1.

216. The composition of claim 211, wherein the composition comprises not less than 0.1% and not more than 1 wt % of Compound 2.

217. The composition of claim 211, wherein the composition comprises not less than 0.1% and not more than 1 wt % of Compound 3.

218. The composition of claim 211, wherein the composition comprises not less than not less than 0.01% and not more than 0.1% of Compound 5a and Compound 5b.

219. The composition of claim 211, wherein the composition comprises not less than not less than 0.01% and not more than 0.1% of Compound 7a and Compound 7b.

220. The composition of claim 211, wherein the total wt % of the one or more compounds is less than or equal to 2 wt %.

221. The composition of claim 210, wherein the composition has aromatase inhibition activity, an affinity to an estrogen receptor, or a combination thereof.

222. The composition of claim 210, wherein the pharmaceutically acceptable salt of the endoxifen is selected from the group consisting of an arecoline, besylate, bicarbonate, bitartarate, butylbromide, citrate, camysylate, gluconate, glutamate, glycollylarsanilate, hexylresorcinate, hydrabamine, hydrobromide, hydrochloride, hydroxynapthanoate, isethionate, malate, mandelate, mesylate, methylbromide, methylnitrate, methylsulfate, mucate, napsylate, nitrate, pamaoate (Embonate), pantothenate, phosphate/diphosphate, polygalacuronate, salicylate, stearate, sulfate, tannate, Teoclate, triethiodide, benzathine, clemizole, chloroprocaine, choline, diethylamine, diethanolamine, ethylenediamine, meglumine, piperazine, procaine, aluminum, barium, bismuth, lithium, magnesium, potassium, and zinc salt.

223. The composition of claim 222, wherein the pharmaceutically acceptable salt of the endoxifen is endoxifen gluconate, endoxifen citrate, or endoxifen hydrochloride.

224. The composition of claim 212, wherein the (Z)-endoxifen is polymorphic Form I of (Z)-endoxifen that is characterized by an x-ray powder diffraction pattern comprising major peaks at 16.8±0.3°, 17.1±0.3°, and 21.8±0.3° two theta.

225. The composition of claim 210, wherein the composition comprises from 0.01 mg to 200 mg of the endoxifen.

226. The composition of claim 210, wherein the composition comprises less than 0.25% water content.

227. The composition of claim 210, wherein the composition comprises less than:
6000 ppm methanol;
9000 ppm ethanol;
9000 ppm acetone;
9000 ppm isopropyl alcohol;
1000 ppm acetonitrile;
9000 ppm ethyl acetate;
1500 ppm tetrahydrofuran;
1500 ppm 2-methyltetrahydrofuran;
252 m benzene;
250 ppm mesityl oxide; or
any combinations thereof.

228. The composition of claim 210, wherein the composition comprises less than:
less than 500 ppm zinc;
less than 500 ppm titanium; or
a combination thereof.

229. The composition of claim 210, wherein the composition is formulated as a tablet or a capsule.

230. The composition of claim 229, wherein the formulation is an enteric formulation.

231. The composition of claim 229, wherein the formulation is a delayed-release formulation.

232. The composition of claim 229, wherein the formulation comprises hydroxypropyl methyl cellulose.

233. The composition of claim 210, wherein the composition is formulated as a pharmaceutical composition comprising the composition and a pharmaceutically acceptable excipient or diluent.

234. The pharmaceutical composition of claim 233, wherein the pharmaceutical composition is administered to a subject in need thereof.

235. The pharmaceutical composition of claim 234, wherein the subject in need thereof has or is at risk of having a hormone-dependent breast disorder, a hormone-dependent reproductive tract disorder, a depression, or any combinations thereof.

236. The pharmaceutical composition of claim 235, wherein the hormone-dependent breast disorder or the hormone-dependent reproductive tract disorder is a benign breast disorder, hyperplasia, atypia, atypical ductal hyperplasia, atypical lobular hyperplasia, increased breast density, gynecomastia, DCIS, LCIS, breast cancer, precocious puberty, McCune-Albright Syndrome, endometrial cancer, ovarian cancer, uterine cancer, cervical cancer, vaginal cancer, or vulvar cancer.

237. The pharmaceutical composition of claim 234, wherein the subject in need thereof has prostate cancer and has or is at risk of having gynecomastia.

238. The pharmaceutical composition of claim 234, wherein the subject in need thereof has tamoxifen-refractory or tamoxifen resistant hormone-dependent breast disorder or hormone-dependent reproductive tract disorder.

239. The pharmaceutical composition of claim 234, wherein the subject in need thereof is or will be treated with an SSRI drug selected from the group consisting of citalopram, escitalopram, fluoxetine, paroxetine, sertraline, and vilazodone.

240. The pharmaceutical composition of claim 234, wherein the subject in need thereof is administered the pharmaceutical composition daily.

241. The pharmaceutical composition of claim 233, wherein the pharmaceutical composition is used in a method of treating a hormone-dependent breast disorder, a hormone-dependent reproductive tract disorder, a depression, or any combinations thereof, the method comprising administration of an effective amount the pharmaceutical composition to a subject in need thereof, thereby treating the hormone-dependent breast disorder, the hormone-dependent reproductive tract disorder, a depression, or any combinations thereof.

242. The pharmaceutical composition of claim 241, wherein the hormone-dependent breast disorder or the hormone-dependent reproductive tract disorder is a benign breast disorder, hyperplasia, atypia, atypical ductal hyperplasia, atypical lobular hyperplasia, increased breast density, gynecomastia, DCIS, LCIS, breast cancer, precocious puberty, McCune-Albright Syndrome, endometrial cancer, ovarian cancer, uterine cancer, cervical cancer, vaginal cancer, or vulvar cancer.

243. The pharmaceutical composition of claim 241, wherein the subject in need thereof is tamoxifen-refractory or tamoxifen resistant.

244. The pharmaceutical composition of claim 241, wherein the subject in need thereof is administered the pharmaceutical composition daily.
